SEQUENCE LISTING (8 sequences)

SEQ ID NO: 1 - DNA, 22 bp, synthetic construct
  source 1..22: mol_type = other DNA
gaagcaggaaaatccctagcag

SEQ ID NO: 2 - DNA, 26 bp, synthetic construct
  source 1..26: mol_type = other DNA
ccaactgaccatccttttccatcttt

SEQ ID NO: 3 - DNA, 22 bp, synthetic construct
  source 1..22: mol_type = other DNA
tcctgagtacggctgagtgaag

SEQ ID NO: 4 - DNA, 27 bp, synthetic construct
  source 1..27: mol_type = other DNA
tcctattcctcctactatttttggggt

SEQ ID NO: 5 - DNA, 27 bp, synthetic construct
  source 1..27: mol_type = other DNA
caggttggccgattctggagtggatgc

SEQ ID NO: 6 - DNA, 23 bp, synthetic construct
  source 1..23: mol_type = other DNA
ctaggggaaggacatggggcagg

SEQ ID NO: 7 - DNA, 25 bp, synthetic construct
  source 1..25: mol_type = other DNA
ccagaacctccactacccattcatc

SEQ ID NO: 8 - DNA, 29 bp, synthetic construct
  source 1..29: mol_type = other DNA
acctccagaaaatgaaggaccacaaaggg

(19) United States
(12) Patent Application Publication
(10) Pub. No.: US 2024/0197712 A1
(43) Pub. Date: Jun. 20, 2024

(54) TGF-BETA INHIBITION FOR IMMUNODEFICIENCY VIRUS REACTIVATION FROM LATENCY, ENHANCEMENT OF IMMUNODEFICIENCY VIRUS-SPECIFIC RESPONSES AND IMMUNODEFICIENCY VIRUS CURE

(71) Applicant: Northwestern University, Evanston, IL (US)

(72) Inventor: Elena Martinelli, Evanston, IL (US)

(21) Appl. No.: 18/515,196

(22) Filed: Nov. 20, 2023

Related U.S. Application Data
(60) Provisional application No. 63/384,368, filed on Nov. 18, 2022.

(51) Int. Cl. A61K 31/4709; A61P 31/18; A61P 37/04

(52) U.S. Cl. CPC A61K 31/4709; A61P 31/18; A61P 37/04

(57) ABSTRACT

Disclosed are methods of treating immunodeficiency virus infection that include methods of treating latent immunodeficiency virus infection in immune cells using a TGF-β inhibitor with antiretroviral therapy.

### CROSS-REFERENCE TO RELATED APPLICATION

This application claims benefit of U.S. Provisional Application Ser. No. 63/384,368, filed Nov. 18, 2022, the entire contents of which is incorporated by reference herein.

### GOVERNMENT RIGHTS

This invention was made with government support under grant AI157822 awarded by the National Institutes of Health. The government has certain rights in the invention.

### REFERENCE TO AN ELECTRONIC SEQUENCE LISTING

The contents of an electronic sequence listing named 702581_02413_SL_ST26.xml (Size 8,272 bytes; and Date of Creation: Nov. 14, 2023) is incorporated by reference in its entirety.

### FIELD OF INVENTION

The disclosed technology is generally directed to methods for improving treatment of immunodeficiency virus infection, including the use of TGF-β inhibition for improving treatment for immunodeficiency virus infection.

### BACKGROUND

Despite the success of combination antiretroviral therapy (ART) in suppressing viral replication and preventing immunodeficiency progression, HIV-1 infection persists and evades immune responses in viral reservoirs, which are considered the major obstacle to HIV-1 cure (1). Long-lived CD4+ T cells that expand by homeostatic or antigen-driven proliferation in blood and tissues constitute a reservoir of proviruses that may be fully latent or transcriptionally active to a different extent in different CD4+ T cells(2, 3). Myeloid cells may also constitute important cellular reservoir(4), although HIV-1 latency in the myeloid compartment has only recently been explored(5, 6). Viral rebound upon ART interruption occurs in the vast majority of patients, likely following stochastic HIV-1 reactivation events and its dynamics depend on the size of the reservoir and host immune factors(7) (8, 9).

“Shock and kill” strategies have the potential to lead to a functional HIV-1 cure by reducing reservoir size and stimulating immune responses(10, 11). However, the success of this strategy is hindered by several obstacles including the vast heterogeneity of the cells comprising the reservoir and of the mechanisms of latency(2, 12) and the low inducibility of the latent proviruses(13, 14). Strategies are needed to address immunodeficiency virus rebound after treatment and viral latency leading to rebound that can improve treatment and provide a cure for immunodeficiency virus infection.

### SUMMARY

An aspect of the disclosure is methods of treating a latent immunodeficiency virus infection in immune cells in a subject receiving antiretroviral therapy comprising administering a TGF-β inhibitor to the subject.

Another aspect is methods for activation of latent immunodeficiency virus in immune cells of a subject having an immunodeficiency virus infection treated with antiretroviral therapy comprising contacting the immune cells with a TGF-β inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It should be understood that the drawings described below are for illustration purposes only and are not intended to limit the scope of the present teachings in any way.

FIGS. 1A-1H. U1 and ACH-2 cells were treated with TGF-β1 (10 ng/ml) or galunisertib (1 μM), both or mock treated in presence vs absence of phorbol 12-myristate 13 acetate-(PMA) (100 ng/ml) for 18 hrs and stained for intracellular p24. (FIG. 1A) An example of p24 detection in one experiment with U1 cells. FIGS. 1B-1C) Raw p24 data from one representative experiment with U1 (FIG. 1B) or ACH-2 (FIG. 1C) cells (black circles: mock; blue squares: TGF-β1; green triangles: TGF-β1+ gal; pink triangles: Gal-only). (FIGS. 1D-1E) Summary fold increase in the frequency of p24+ cells over the mock condition (box plot with median and min max whiskers) are shown from 5 similar experiments (U1 on the left; ACH-2 on the right). (FIGS. 1F-1G) For this primary CD4+ T cell model of latency, CD4+ T cells were isolated from PBMC, activated, infected by spinoculation and incubated for 2 days in presence of T20. PMA (100 ng/ml) was used for reactivation of latently infected cells for 18 hrs in presence of TGF-β1 (10 ng/ml) or galunisertib (1 μM), both or mock treatment. A schematic of the experiment is shown in (FIG. 1F). (FIG. 1G) Summary data (fold increase over the unstimulated condition) from 5 experiments with cells from different donors run in triplicate. (FIG. 1H) Data from a model of DC-driven HIV reactivation from U1 cells are shown. Fold difference in the frequency of p24+U1 cells in absence vs presence of moDCs or TGF-β DCs are shown for 5 different experiments in triplicate (box plot with median and min max whiskers). (FIGS. 1A-1H) Conditions were compared by Kruskal-Wallis ANOVA test followed by the Dunn's test corrected for multiple comparisons (Significant p-values of α<0.05 (*), α<0.01 (*) and α<0.001 (*) are indicated. All other comparisons were non-significant). Box and whisker plots represent median, 25th and 75th percentile with whisker going from min to max.

FIG. 2. TGF-β inhibits HIV-1 latency reactivation by PMA in ACH-2 cells. Flow cytometry multicolor plots of p24 ICS from one experiment with ACH-2 cells treated with TGF-β1 (10 ng/ml) or galunisertib (GAL; 1 μM) or both overnight.

FIGS. 3A-3C. More profound effect of TGF-β in absence of FBS. (FIG. 3A) Flow cytometry multicolor plots of p24 ICS from one representative replicate of U1 cells treated with PMA in X-Vivo 15 media without FBS and TGF-β1 (10 ng/ml) or galunisertib (1 μM) or both overnight. (FIGS. 3B-3C) Experiments in triplicate with U1 (FIG. 3B) or ACH-2 (FIG. 3C) cells cultured in X-Vivo 15 media without FBS and treated with TGF-β1 (10 ng/ml) or galunisertib (1 μM) or both overnight. Brwon-Forsythe and Welch Anova with Dunnett's T3 correction for multiple comparisons was used to compare the different treatments. (Significant p-values of α<0.05 (*), α<0.01 (**) and α<0.001 (***) are indicated).

FIG. 4. TGF-β inhibits HIV-1 latency reactivation by PMA in primary CD4+ T cells. Flow cytometry multicolor plots of p24 ICS from one representative experiment of primary CD4+ T cells model of HIV-1 latency. Freshly isolated CD4+ T cells were activated and infected with HIV-ADA by spinoculation. After 48 hrs, cells were stimulated with PMA in presence vs absence of TGF-β1 (10 ng/ml) or GAL(1 μM) or both overnight.

FIGS. 5A-5B. TGF-β-moDCs reactivate latent HIV-1 less than untreated moDCs. (FIG. 5A) High-low box plots (line at median) of Log MFI of expression of the indicated marker on moDCs or TGF-β-moDCs from 3 different donors are shown. Paired t-test was used to compare moDCs and TGF-β-moDCs (Significant p-value of α<0.05 (*) is indicated). (FIG. 5B) One representative example of multicolor plots from a p24 ICS of eFluo670-labelled U1 cells co-cultured with moDCs or TGF-β-moDCs overnight.

FIGS. 6A-6C. Blocking TGF-β1 signaling increases LRA-induced HIV-1 reactivation ex vivo. De-identified PBMC from aviremic, ARV-treated PLWH collected at NIH and NU were used for viral outgrowth assays to estimate the frequency of HIV infected cells able to produce replication competent virus within the unfractionated PBMC. (FIG. 6A) Schematic of the qVOA assay where activation with a latency reversal agent (LRA; namely PMA or Vorinostat) was followed by collection of supernatant for vRNA quantification (n=5) and co-culture with SupT-1 cells (n=9) in replicate wells (>14 wells). (FIG. 6B) The IUPM calculated based on the frequency of p24+ wells in each condition are shown (pink squares represent PBMC from NIH, triangles represent PBMC from Northwestern, RADAR). (FIG. 6C) vRNA-gag copies/ml of culturing media in presence vs absence of galunisertib (1 μM). Conditions were compared by a 2-way ANOVA followed by the Sidak multiple comparisons test (Significant p-values of α<0.05 (*), α<0.01 (**) and α<0.001 (**) are indicated. All other comparisons were non-significant).

FIGS. 7A-7B. Galunisertib does not impact in vitro HIV-1 replication. The effect of galunisertib on HIV replication was tested in SupT1-M10 and PBMC from HIV− donors infected at low MOI to detect an increase in replication. One of 2 experiments in triplicates in SupT1 (FIG. 7A) and PHA-PBMC (FIG. 7B) infected with HIV Bal at MOI of 0.01+ Gal (added every 2 days).

FIGS. 8A-8G. Blocking TGF-β1 leads to HIV-1 reactivation in vivo. (FIG. 8A) Schematic representation of the macaque studies. SIVmac251 and SIVmac239 infected macaques (n=7) were treated with ARVs starting week 12 post infection. After 26-27 weeks of ART, animals were treated twice daily with 5 or 10 mg/kg of galunisertib orally. Colorectal biopsies, fine needle aspirates (FNA) and PET/CT scans with the anti-SIV-env probe 64Cu-7D3 were performed before and at the end of the 1 or 2 weeks treatment. The animals that were treated for 2 weeks underwent a 2nd scan at the end of the 1st week of treatment. (FIG. 8B) Plasma viral loads measured at NIRC (LLOQ=100 copies/ml). Gal was administered every day (twice daily) for 1 or 2 weeks. Timing of Gal administration is represented by pink rectangles above the pVL. (FIGS. 8C, 8D, 8E) Representative images from the PET/CT scans of 4 out of 5 animals with increased PET signal following galunisertib treatment. (FIGS. 8F-8G) SUVtot for different anatomical areas (ROI) are shown before and after galunisertib treatment (Gut includes small and large intestine, LN=axillary LNs, NALT=nasal associated lymphoid tissues, SPL=spleen). Data from BL and post-galunisertib W1/2 were compared using Wilcoxon matched-pairs non-parametric test and the differences were non-significant with α>0.05.

FIG. 9. List of macaques used in the treatment and PK studies.

FIGS. 10A-10B. Galunisertib PK and PD in rhesus macaques. (FIG. 10A) 8 naïve rhesus macaques were treated with galunisertib only once (n=4) or twice per day for 9 days and plasma collected at the indicated time (hrs) after the first dose of galunisertib. Galunisertib concentrations in plasma samples were measured by LCMS. (FIG. 10B) PBMC from 4 animals treated for 9 days with galunisertib at 2.5 mg/kg or 5 mg/kg twice daily were anayzed for phosphoSMAD2/3 amounts and total pSMAD2/3. The percentage ration of Phospho over Total-SMAD2/3 is shown at the indicated time point after the first galunisrtib treatment.

FIG. 11. Plasma VL during galunisertib treatment as detected by Leidos. Plasma samples collected before, during and after the 7 or 14 days treatment with galunisertib were assayed for plasma viral loads at Leidos with an ultrasenstitive (LLOD 3 to 5 copies/ml) assay and the results shown here. Pilot 1 indicates the study with 4 animals that were treated for 7 days. Pilot 2 indicates the study with 3 animals that were treated for 14 days.

FIG. 12. A control IgG1 probe accumulates in liver and kidneys with no binding in tissues. A ⁶⁴Cu-DOTA-F(ab′)2 pIgG1 control Fab probe was generated by digestion of the Fc portion of a rhesus isotype control IgG1 obtained from the NHP reagent repository. MIPs were generated using the MIM software and set to a numerical scale of 0-1.5 SUVbw visualized with the color scale PET Rainbow (same settings used for the galunisertib treated animals). The PET signal accumulates as expected in the liver, kidney and spleen.

FIGS. 13A-13C. PET SUVtot in a specific tissue area correlated with CA-vRNA levels. A piece of small intestine was collected during a major resection surgery after being identified as positive during a whole-body PET/CT scan of an SIV infected macaque (08M171). Adjacent MLN were also collected. (FIG. 13A) The tissue and MLN were placed on a petri dish and a picture taken before re-scan. The picture of the tissue on the petri dish was overlapped to the PET image and the tissue sectioned in 2 based on the level of PET signal along the white dotted line as shown. Each piece was snap frozen and tested for CA-vDNA/RNA by qPCR. (FIG. 13B) The levels of CA-vDNA in each tissue area are shown. (FIG. 13 C) The correlation between SUVtot in each tissue area and CA-vRNA levels are shown.

FIGS. 14A-14E. Increased PET-signal corresponds to increased vRNA. (FIG. 14A) Copies of cell-associated unspliced vRNA normalized on 10\hat{ }6 cells diploid genome equivalent are shown for colorectal biopsies and LNA before and after galunisertib treatment. Data from BL and post-galunisertib W1/2 were compared using Wilcoxon matched-pairs non-parametric test and the differences were not significant with α>0.05. (FIG. 14B-14C) The correlation between the fold increase in SUVtot for the gut (FIG. 14B) and LN (FIG. 14C) and the fold increase in vRNA copies in rectal biopsies and FNA (only 1 of the axillary LNs was sampled) are shown. Pearson's correlation coefficient and p values are indicated in each graph. (FIG. 14D) Copies of vDNA per 10⁶ cells-equivalent are shown for the PBMC of the 3 animals that were treated with galunisertib for 2 weeks. W3 time point represents a sample collected 7 days after the last galunisertib administration. No statistical test was performed. (FIG. 14E) Intra-host viral quasispecies evolution before and after galunisertib treatment in A14X013 is shown. Inferred ML tree with the reconstructed the intra-host quasispecies in PBMCs, rectal tissues, and blood samples using deep sequencing of the gag gene. Circles at the tips represent each haplotype and are colored by time post-infection and sample type (D84 is plasma right before ART initiation; D273 is PBMC on the day of first galunisertib treatment, collected before treatment; D280-287-290 represent 7, 14 and 20 days after galuniertib initiation). Circle size indicates the frequency of the haplotype in the quasispecies. Insert represents the spatio-temporal evolution of the viral population diversity at each compartment and time point. Both y-axis and circle size indicate diversity of quasispecies and data from the same sample type are connected by lines. Intra-host viral diversity was calculated as weighted average of pairwise distances between every haplotype weighted by their frequency in the population in substitutions per site.

FIGS. 15A-15B. Ratio vRNA/DNA in colorectal and lymph node biopsies. (FIG. 15A) Copies of SIV-gag DNA per cell equivalent are shown for colorectal biopsies and lymph nodes (FNA) collected before and after galunisertib treatment. (FIG. 15B) The ratio of vRNA copies/vDNAcopies for each tissue is shown. In the LNs the ratio is 1 and unchanged for 4 of the 7 animals and the lines overlap each other. Differences between BL an W1/2 are not significant by Wilcoxon matched-pairs test.

FIGS. 16A-16B. TGF-β levels do not predict the effect of Gal on the viral reservoir. (FIG. 16A) The concentration of total TGF-β in the plasma before and after galunisertib as detected by ELISA is shown. Wilcoxon matched-pairs test was used to compare BL with W1/2 data, Significant p-value of α<0.05 (*) is shown. (FIG. 16B) No correlation is present between the concentration of TGF-β before treatment in blood and the change in Gut-SUVTot in each animal after galunisertib treatment (p=0.199; Person R=0.304).

FIGS. 17A-17E. Galunisertib treatment stimulates immune cell pathways. (FIG. 17A) CD4+ T cells were isolated from PBMC of naïve macaques before and after (6 hrs) treatment with galunisertib for PK studies (see FIG. 7) and TotalRNAseq was performed at the Northwestern University Center for Genetic Medicine NUSeq core. Data were analyzed and genes were classified as down-regulated genes (FIGS. 17B-17C) or up-regulated genes (FIG. 17D-17E).

FIGS. 18A-18B. Blocking TGF-β stimulates SIV-specific responses. (FIG. 18A) The frequency of CD4+ T cells and CD8+ T cells that express the cytokines indicated below the graphs in response to stimulation with a pool of 15-mer overlapping peptides from SIVmac239 gag and env are shown for before and after galunisertib treatment. The frequency of each population was calculated after subtraction of a DMSO unstimulated control performed in parallel with each sample. A mixed-effect model with the Sidak multiple comparisons test, with a singled pooled variant was used to test for significant differences (Significant p-values of α<0.001 (***) and α<0.0001 are indicated. Only IFN-γ was significant with this test. Non-significant differences in all other cytokine combinations are not indicated). (FIG. 18B) SIV-env titers in plasma before and after galunisertib treatment are shown for each animal. Wilcoxon matched pairs non-parametric t-test was used to compare before and after galunisertib (Significant p-values of α<0.05 (*), α<0.01 (*) and α<0.001 (*) are indicated).

FIG. 19 is a Heat map of top 50 metabolites blocking TGF-β1 decreases TCA cycle cell-associated metabolites in PBMC. Semi-quantitative comparison of metabolites detected by LCMS in PBMC from SIV infected, ART treated macaques before and after TGF-β inhibitor.

FIGS. 20A-20E illustrate four 2-week cycles with galunisertib lead to viral reactivation in blood. (FIG. 20A) Schematic representation of the study and sampling schedule (FIGS. 20B-20D) Plasma VL in blood for each macaque throughout the study. The longer black line indicates the period on ART, while the 4 small black lines indicate the start and end of each galunisertib cycle. (FIG. 20E) Enlarged plasma VL for all macaques during galunisertib therapy. Green bars indicate galunisertib cycles.

FIGS. 21A-21G. Galunisertib leads to viral reactivation in tissues. (FIG. 21A) The ⁶⁴Cu-DOTA-Fab2(7D3) probe was injected ˜24 hrs before PET/CT scan before and at the end of each of the first 3 galunisertib cycles. Representative images from the PET/CT scans are show for a macaque with a major increase at the end of cycle 2 and one showing increase at the beginning of cycle 3. (FIGS. 21B-21G) Total SUV were calculated for each anatomical area and values analyzed with mixed-effect analysis. Thicker black line represents the mean. P-values were calculated for comparison of each time point with the before cycle 1 time point (BC1; AC1=after cycle 1, BC2=before cycle 2; AC2=after cycle 2; BC3=before cycle 3; AC3=after cycle 3; Holm-Sidak multiple comparison correction; *p≤0.05 **p≤0.01 ***p≤0.01).

FIGS. 22A-22C. Representative images from the PET/CT scans performed in all macaques before and after each galunisertib cycle that leads to viral reactivation in tissues. (FIGS. 22A-22C) not shown in FIGS. 20A-20E.

FIG. 23. Total SUV for the whole body. Total SUV were calculated for whole-body after normalization and values analyzed with mixed-effect analysis. P-values were calculated for comparison of each time point with the before cycle 1 time point (BC1; AC1=after cycle 1, BC2=before cycle 2; AC2=after cycle 2; BC3=before cycle 3; AC3=after cycle 3; Holm-Sidak multiple comparison correction; *p≤0.05 **p≤0.01 ***p≤0.01).

FIGS. 24A-24B. Relationship between vRNA and SUV in the gut. Total SUV for the gut area are plotted together with cell-associated vRNA in colorectal biopsies at the specified time during galunisertib treatment (BC1=before cycle 1, AC1=after cycle 1, BC2=before cycle 2; AC2=after cycle 2; BC3=before cycle 3; AC3=after cycle 3).

FIGS. 25A-25C. Galunisertib decreases viral reservoir in absence of systemic inflammation. (FIG. 25A) Levels of cell-associated (CA)-vDNA per cell equivalent are shown for the time point before cycle 1 (BC1) and at the end of cycle 3 (AC3) or 4 (AC4) for the respective tissues. (FIG. 25B) IPDA data are shown for intact and total provirus for BC1 and AC4 in PBMC. pvalues are shown for Wilcoxon matched pair signed-rank non-parametric test (*p≤0.05 **p≤0.01 ***p≤0.01) (FIG. 25C) Heat map of cytokine concentration in plasma at the indicated time point are shown after Log transformation and normalization. Statistical analysis was run on each factor separately and together (no significant differences after multiple comparison adjustment).

FIGS. 26A-26C. Galunisertib decreases CA-vDNA with a minor increase in systemic IL-10. (FIG. 26A) Levels of cell-associated (CA)-vDNA per cell equivalent are shown for the pre-ART time point (week 6) and for all the time points before and after the first 3 galunisertib cycles (BC1, AC1, BC2, AC2, BC3, AC3) for PBMC and also for after cycle 4 (AC4) for Gut and LN. pvalues are shown for a mixed effect model and post-hoc Holm-Sidak correction (*p≤0.05 **p≤0.01 ***p≤0.01) BC3 had only one value in the LN (08M171) and was not included in the analysis. (FIG. 26B) CA-vDNA in PBMC from 4 SIVmac239M2 infected, ART-treated macaques not treated with galunisertib at week 28 vs week 52 post-infection. (FIG. 26C) IL-10 levels in plasma at the indicated time point. pvalues are shown for a mixed effect model and post-hoc Holm-Sidak correction (*p≤0.05).

FIGS. 27A-27K. Galunisertib leads toward effector T and NK cells, increasing Treg and decreasing Tfh frequencies. (FIGS. 27A-27F) Geometric mean fluorescent intensities (MFI) of each marker and frequency of indicated subset within live, singlets CD3+CD4+ T cells (FIG. 27A and FIG. 27C) or CD3+CD4+CD95+ T cells (FIG. 27B) or CD8+ or CD8+CD95+ T cells or NK cells (NKG2A+CD8+CD3− cells) are shown. Thick black line represents the mean. Changes from baseline (beginning of cycle 1, BC1) are shown for graphs with at least 1 significant difference (Repeated measures ANOVA with Holm-Sidak correction; *p≤0.05 **p≤0.01 ***p≤0.01). (FIGS. 27G-27H) tSNE of lymphocyte, live, singlets events after normalization for BC1 and AC4 (all 8 macaques) with FlowSOM 36 clusters overlaid on tSNE (FIG. 27G, top) or heatmap of each markers MFI (FIG. 27H) or heatmap of time point (blue is BC1 and red is AC4; FIG. 27G, bottom) is shown. 6 populations were manually gated on red or blue areas (red, New1-3 and blue Old1-3). (FIG. 27I) Bubble chart displaying changes from BC1 in AC4. Color is proportional to the effect size and size to p-value (Wilcoxon sum rank non parameter test). (FIG. 27J) Bubble chart displaying changes from BC1 in all time point (ANOVA repeated measures with Holm-Sidak correction; *p≤0.05 **p≤0.01 ***p≤0.01). (FIG. 27K) Frequency of indicated subset within live, singlets CD3+CD4+T or CD3+CD4+CD95+CD28+ T cells (CM=central memory) or within CD3+CD8+ T cells. Changes from baseline (BC1) are shown (ANOVA repeated measures with Holm-Sidak correction for multiple comparisons; *p≤0.05 **p≤0.01 ***p≤0.01).

FIGS. 28A-C present a gating strategy for classical analysis of 24-color flow (full strategy presented across three images; FIG. 28A rows 1 to 2; FIG. 28B rows 2 to 3, and FIG. 28C rows 3 to 4).

FIGS. 29A-29B illustrate decrease in integrin α4β7, but no change in classical activation or exhaustion markers after galunisertib treatment. Geometric mean fluorescent intensities (MFI) of each marker and frequency of indicated subset within live, singlets CD3+CD4+ T cells (FIG. 29A), or within CD3+CD8+ T cells (FIG. 29B), or CD4+CD95+ T cells (FIG. 28A where indicated on Y label) are shown. Thick black line represents the mean. Changes from baseline (beginning of cycle 1, BC1) are shown only for graphs with at least 1 significant difference (One-way Anova for repeated measures with Holm-Sidak correction for multiple comparisons; *p≤0.05 **p≤0.01 ***p≤0.01). Of note, drop in PD1 expression in cycle 3 for 08M156 and A6X003 is due to in vivo treatment with aPD1 Ab. Decline in PD1 is not significant after exclusion of the cycle 3 time point in these 2 macaques.

FIGS. 30A-30E show heatmaps of populations generated by FlowSOM. (FIG. 30A) FlowSOM was run with phenograph suggested 36 populations on cleaned, live, singlet lymphocytes of BC1 and AC4 data. Heatmap of MFI for each marker in each FlowSOM population is shown. (FIGS. 30B-30D) tSNE visualization of lymphocyte, live, singlets events after normalization for all time points analyzed (BC1, AC1, BC2, AC2, BC3, AC3 and AC4 for all 8 macaques) with FlowSOM 38 clusters overlaid on the tSNE (FIG. 30B, top) or heatmap of each markers MFI (FIGS. 30C-30D) or heatmap of time point (blue is BC1 and the progressively more toward red AC4; FIG. 30B, bottom) is shown. (FIG. 30E) FlowSOM was run with phenograph suggested 38 populations on cleaned, live, singlet lymphocytes of all time points. Heatmap of MFI for each marker in each FlowSOM population is shown.

FIG. 31 illustrates a gating strategy for analysis of Tfh and Treg frequencies.

FIGS. 32A-32K. OXPHOS and other metabolic pathways increase rapidly with TGF-βblockade. Bulk RNAseq was performed with PBMC from before cycle 1 (24 hrs) and 1 hrs after the first dose of galunisertib (FIGS. 32A32-D), or after the last dose of cycle 1 (FIG. 32E-32H) and with rectal biopsies collected before cycle 1 (24 hrs) and after the last dose of cycle 1 (FIG. 32I-32K). The number of differentially expressed genes (DEG) with an Adjpvalue <0.05 and log 2FC>2 and <−2 are shown in each respective volcano plot. Enrichment plots are shown after GSEA (with all adjp<0.05 DEGs) for significantly enriched pathways (top 1 or 2 pathway by ES). (FIG. 32K) Lollipop graph of selected DEG of interest among significantly different genes (adjp<0.05).

FIGS. 33A-33D. DEG and pathways enriched 1 hr after galunisertib in PBMC. (FIG. 33A). Heatmap of genes in the Hallmark OXPHOS pathways upregulated (red) or downregulated (in each monkey) 1 hr after the first dose of galunisetrib. (FIG. 33B). DEGs of interest (upregulated left and downregulated right after 1 hr). (FIG. 33C) Metascape analysis of hallmark gene sets enriched in upregulated DEGs after 1 hr of galunisertib. (FIG. 33D) List of top 20 DEGs (for log 2FC) upregulated or downregulated after 1 hr of galunisertib.

FIGS. 34A-34D. DEG and pathways enriched after 2 weeks of galunisertib in PBMC. (FIG. 34A) Heatmap of genes in the Hallmark OXPHOS, Myc_Targets_V1 and KREB pathways upregulated (red) or downregulated (in each monkey) after 2 weeks of galunisetrib. (FIG. 34B) DEGs of interest (upregulated left and downregulated right after 2 weeks). (FIG. 34C) GSEA enrichment plot and heatmap of genes of mTORC1 pathway enriched among hallmark sets. (FIG. 34D) List of top 20 DEGs (for log 2FC) upregulated or downregulated after 2 weeks of galunisertib.

FIGS. 35A-35I. scRNAseq of lymph node before and after cycle 1 confirms a switch toward effector and increased metabolism in all immune subsets with galunisertib. (FIG. 35A) UMAP projection of 93234 cells from lymph nodes collected right before and at the end of cycle 1 from all 8 macaques (16 samples). Gene-based classification of major immune subset is overlaid on UMAP. In gray are unclassified cells. (FIGS. 35B-35C) Bubble plots showing expression (mean normalized counts proportional to the color; size proportional to the percentage of cells) of each marker listed in each cell subset. Marker listed are those used for classification of major immune subsets (FIG. 35B) or CD4+ and CD8+ T cells (FIG. 35C). (FIG. 35D) Significantly different genes (Adjp<0.05; log 2FC=0.15) in the T cell subset are shown with color proportional to normalized counts. (FIG. 35E) Significantly enriched pathways (adjp<0.01) in T cells DEGs within the hallmark collection. (FIGS. 35F-35G) Significantly different genes (Adjp<0.05; log 2FC=0.15) in the CD4+(FIG. 35F) and CD8+(FIG. 35G) T cell subset. (FIGS. 35H-35I) Significantly enriched pathways (adjp<0.01) in CD4+ T cells DEGs within the hallmark (FIG. 35H) and biocarta (FIG. 35I) collections.

FIGS. 36A-36C. UMAP and heatmap of cell classification for scRNAseq cells from lymph nodes collected right before and after galunisertib cycle 1. (FIGS. 36A-36B) UMAP projection of 93234 cells from lymph nodes collected right before and at the end of cycle 1 from all 8 macaques (16 samples). Gene-based classification of CD4 and CD8 T subset (FIG. 36A) and B subsets and Tfh (FIG. 36B) is overlaid on UMAP. In gray are unclassified cells. (FIG. 36C) Bubble plot showing expression (mean normalized counts proportional to the color; size proportional to the percentage of cells) of each marker listed in each cell subset. Marker listed are those used for classification of major immune subsets of B cells and Tfh cells.

FIGS. 37A-37H. Frequency of each population within LN before and after cycle 1 with galunisertib. Cells were classified using gene expression and the number of cells of a specific type was normalized over the total cell number for each sample: B cells (FIG. 37A), T cells (FIG. 37B), NK cells (FIG. 37C), macrophages (FIG. 37D), CD4 T cells (FIG. 37E), Naïve B cells (FIG. 37F), Tfh (FIG. 37G), and GC B cells (FIG. 37H). Wilcoxon signed rank test was used to compare frequencies before and after galunisertib (no significant differences p<0.05 were found).

FIGS. 38A-38F. Metabolic processes are upregulated in both T and B cells after galunisertib in the LN. (FIG. 38A) Pathways enriched in T cells among gene ontology cellular processes collection are listed with ES>100. (FIG. 38B) Pathways enriched in CD8 T cells among hallmark sets. (FIG. 38C) Hallmark pathways enriched (adjp<0.05) and (FIG. 38D) DEGs in Tfh cells (log 2fc 0.15). (FIG. 38E) DEG in B cells with log 2fc 0.2 cutoff. (FIG. 38F) Hallmark pathways enriched in B cells.

FIGS. 39A-39D. Galunisertib leads to a switch towards effector/inflammatory phenotype in B cells, NK, and macrophages. (FIGS. 39A-39B) Significant DEGs with a log 2FC cut off 0.15 in GC B cells and naïve B cells. (FIG. 39C) Significant DEG in NK cells with a cutoff log 2FC=0.3. (FIG. 39D) Significant DEG in Macrophages with a cutoff log 2FC=0.25.

FIGS. 40A-40B. Galunisertib increases SIV-specific responses. Average spots (from triplicates) per 106 PBMC at the time of ART initiation (pre-ART), before cycle 1 (BC1), after cycle 1 (AC1) and at the end of cycle 3 (AC3) with galunisertib after 24 hrs ex vivo stimulation with 15-mer peptides (gag, env, pol) or mock (DMSO). Each post-galunisertib time point was compared to BC1 (Mixed effect analysis with Dunnet post-hoc p-values are shown; *p≤0.05 **p≤0.01 ***p≤0.001).

FIGS. 41A-41J. No changes in barcode numbers with galunisertib, but significant changes in barcode distribution in different tissues. (FIG. 41A) Number of barcodes detected before (blue) and after (red) each galunisertib cycle in the listed tissues. (FIGS. 41B-41J) Barcode frequency distribution in each monkey and tissue in which virus was detected. Each barcode has a different color, and its bar size is proportional to its frequency. Data from before and after each of the first 3 galunisertib cycles (FIGS. 41B-41I) or pre-ART vs post-ATI week 6 (FIG. 41J) are shown.

FIGS. 42A-42E. TCF1 decrease associates with virological and immunological endpoints. (FIG. 42A) Barcode diversity measure as Shannon Entropy is shown before and after each of the first 3 galunisertib cycles for LN, PBMC and colorectal biopsies. Box-and-whisker plot represents the median+/− the interquartile range (no significant differences using linear mixed effects models). Blue=before; Red=end of each cycle. (FIG. 42B) Bubble plot shows the results of statistical testing (Chi-squared) for differences in frequency distribution of barcodes before compared to after, for each of the first 3 cycles of galunisertib for each macaque in the indicated tissues. Blue indicates significant differences p≤0.05. (FIG. 42C) Barcode entropy of virus isolated at the time of ART initiation compared to week 6 post-ATI in plasma (Wilcoxon matched pairs test; *p≤0.05). (FIG. 42D) Correlation matrix of several key variables of virological or immunological effect of galunisertib. Color is proportional to Pearson r coefficient. *p≤0.05 **p≤0.01 ***p≤0.001 indicate significant correlations. (FIG. 42E) Association between fold increase in TCF1 (MFI) from BC1 to AC4 with CA-vDNA levels at AC4, change in gut SUV at AC3 compared to BC1 and fold increase in IFN-γ (AC3 vs BC1). Person r is shown. All correlations have*p≤0.05.

FIGS. 43A-43C. Correlations between changes in TCF1, Gut SUV, and intact pro-virus with virological and immunological parameters. (FIG. 43A) Fold changes in TCF1 MFI are plotted against CA-vDNA copies in PBMC at the end of cylcle 3 and CA-vRNA copies in LN at the end of cycle 4 (AC4). Pearson r is shown (FIG. 43B) Fold changes in gut SUV are plotted against CA-vDNA in PBMC at the end of cycle 3 and in rectal biopsies and lymph nodes at the end of cycle 4. Pearson r is shown. (FIGS. 43A-43B) Significant correlations p<0.05 (FIG. 43C) Fold changes in intact pro-virus are plotted against the avg spot for IFN-g after stimulation with SIV-peptides (env+gag+pol). Pearson r is shown. p=0.117.

### DETAILED DESCRIPTION

Disclosed are methods of treating immunodeficiency virus infection in a subject using an inhibitor of TGF-β and TGF-β signaling in cells. TGF-β is known to regulate diverse cellular processes, including proliferation, differentiation, and migration. High concentrations of TGF-β have been reporting in the blood, lymphoid tissues, and cerebrospinal fluid of subjects with immunodeficiency virus infection, both human immunodeficiency virus (HIV) and simian immunodeficiency virus (SIV). Aspects of this disclosure are methods of improving treatment of immunodeficiency virus infection by administering an inhibitor of TGF-β to a subject that can activate immune cells and induce latency reversal among other effects. Such effects can contribute to or lead to improved treatment or cure of immunodeficiency virus infection.

Transforming growth factor beta (TGF-β) is a multifunctional cytokine belonging to the transforming growth factor superfamily that includes three different mammalian isoforms (TGF-β 1, 2, and 3, TGF-β1, TGF-β2, TGF-β3). TGF-β is one of the most potent endogenous immunosuppressive factors, and it is considered the master regulator of mucosal immunity. TGF-β signaling regulates diverse cellular processes, including proliferation, differentiation, and migration, and influences developmental as well as pathological processes such as epithelial-mesenchymal transition in fibrosis. In the immune system, TGF-β is essential to the maintenance of tolerance. Importantly, TGF-β is the most prominent factor responsible for the maintenance of a resting state in CD4+ T cells, and loss of TGF-β responsiveness in mature T cells decreases their threshold for activation. Moreover, TGF-β also affects activation status and function of myeloid cells by decreasing antigen presentation capability and maturation and inducing tolerogenic properties.

TGF-β plays a critical role in maintaining immune cells in a resting state by inhibiting cell activation and proliferation. Resting immunodeficiency virus target cells (e.g. HIV and SIV target cells) represent the main cellular reservoir of virus, provirus, and viral components, particularly after long-term antiretroviral therapy (ART). Immunodeficiency virus target cells may be any immune cells, such as T cells, B cells, NK cells, and macrophages. Given the suppressive activity of TGF-β on different immune cell targets of HIV or SIV infection and its role in latency induction, TGF-β was investigated and found to have a suppressive effect on latency reactivation in virus infected cells. Moreover, based on TGF-β inhibition of T cell receptor (TCR)- or protein kinase C (PKC)-driven HIV reactivation from latency, inhibition of TGF-β signaling is identified to increase the frequency of latency reversal in infected cells. An aspect of the disclosure is methods of treating a latent immunodeficiency virus infection in immune cells in a subject by administering a TGF-β inhibitor to the subject. One aspect is a method of treating a latent immunodeficiency virus infection in immune cells in a subject receiving antiretroviral therapy by administering a TGF-β inhibitor to the subject. In embodiments, the TGF-β inhibitor activates or reactivates the latent immunodeficiency virus in the infected immune cells. In embodiments, the TGF-β inhibitor stimulates immune cells. The stimulated immune cells may be effective in suppressing or eliminating the virus that has been activated or reactivated.

The term “latent,” as used in this disclosure in the context of a latent immunodeficiency virus refers to a genomically integrated immunodeficiency virus (including a latent immunodeficiency virus-based retroviral vector, e.g., a recombinant immunodeficiency virus) that is transcriptionally silent or inactive, e.g., immunodeficiency virus transcripts are undetectable or are at background levels, in a cell comprising the latent immunodeficiency virus.

“Virus latency” or “viral latency” is the ability of a pathogenic virus to lie dormant (latent) within a cell, such as an immune cell. A “latent infection” is an infection by an organism or infectious agent in the body, such as in cell, that remains inactive in the body, without causing any symptoms of the infection.

The term “subject” may be used interchangeably with the terms “individual” and “patient” and includes human and non-human mammalian subjects. A “subject in need” as used in this disclosure may refer to a subject in need of treatment for a disease or disorder, such as a disease or disorder associated with a viral infection. A subject in need thereof may include a subject infected with immunodeficiency virus, including human immune deficiency virus (HIV) 1 or 2. A subject may also include non-human mammals infected with immunodeficiency virus, including but not limited to simian immunodeficiency virus (SIV) or feline immunodeficiency virus (FIV). Viral infection, including HIV may be detectable by any means known in the art including but not limited to detection via antibody tests, antigen test, or nucleic acid tests.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

In aspects of the disclosure, small molecule TGF-β inhibitors and anti-TGF-β antibodies that block TGF-β are used in the methods. TGF-β inhibitors may include those that block, prevent, or diminish TGF-β function, action, signaling or expression. TGF-β is a membrane-bound TGF beta receptor protein of the TGF-beta receptor family for the TGF beta superfamily of signaling ligands. The three isoforms of TGF-β (TGF-β1, TGF-β2, and TGF-β3) bind to the specific type I receptor TGFBR1/ALK5, which then undergoes dimerization with the TGF-β type 2 receptor (TGF-βRII) Of the isoforms, TGF-β1 appears to be the most prominent regulator of immune responses. In the embodiments, TGF-β inhibitors are used in the disclosed methods.

In some aspects, TGF-β type 1 receptor (TGF-βR1) inhibitors are used. Galunisertib, also known as LY2157299 (CAS No. 396129-53-6) is a selective, ATP-competitive inhibitor of Transforming Growth Factor-β Type I receptor kinase. Galunisertib is a small-molecule selective inhibitor of the TGF-β type I receptor TGFBR1/ALK5 (41, 42), which was developed as an anti-cancer therapeutic to target the direct and indirect effect of TGF-β on tumor growth. Galunisertib may also serve as a TGF-β inhibitor in immunodeficiency virus infection. In embodiments, the TGF-β inhibitor, such as TGF-βR1 inhibitor used in the methods is Galunisertib.

Immunodeficiency virus infection, such as HIV, is well treated with compounds, compositions, or easy-to-take medications referred to as antiretroviral therapy (ART). Immunodeficiency virus, however, persists in some immune cells (e.g. T cells) throughout the body during ART. These immune cells either make pieces of the virus (without making infectious viruses that can spread to other cells) or they harbor viruses that are completely quiescent and not making any virus components (latent reservoir). At least some of these cells in each person with HIV infection remain capable of making infectious virus.

In embodiments, a TGF-β inhibitor may be administered with antiretroviral therapy. The TGF-β inhibitor may be administered before, during or after the administration of antiretroviral therapy. Antiretroviral therapy is treatment of subject infected with immunodeficiency virus (e.g. HIV or SIV) using anti-HIV (SIV) drugs. Antiretroviral therapy reduces the amount of virus in a subject. Antiretroviral therapy suppresses or eliminate active immunodeficiency virus in the subject. The standard treatment consists of a combination of drugs (often called “highly active antiretroviral therapy” or HAART) that suppress HIV replication. Antiretroviral therapy includes but is not limited to, abacavir (Ziagen), emtricitabine (Emtriva), lamivudine (Epivir), tenofovir disoproxil fumarate (Viread), and zidovudine (Retrovir).

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results. An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

The agents described herein may be administered alone or in combination, one or multiple times, simultaneously or not. In aspects of the disclosed methods, such methods of treating a latent immunodeficiency virus infection in immune cells in a subject receiving antiretroviral therapy by administering a TGF-β inhibitor to the subject, the antiretroviral therapy suppresses active immunodeficiency virus in the subject. In embodiments, the TGF-β inhibitor is administered after the antiretroviral therapy suppresses active immunodeficiency virus in the subject. In embodiments, the TGF-β inhibitor may be administered following a period of sustained ART, and the TGF-β inhibitor administered for a continuous period of time, or intermittent period of time. A subject may be monitored and/or tested for viral infection during the treatment period, and the treatment schedule adjusted based on the results of testing during the treatment period.

In embodiments, the TGF-β inhibitor activates the latent immunodeficiency virus in the infected immune cells. In embodiments, the TGF-β inhibitor reduces immunodeficiency provirus in the immune cells infected with the virus.

In a further aspect, the TGF-β inhibitor stimulates immune cells to effector immune cells in the subject. In embodiments, the effector immune cells are T cells, B cells, NK cells, and/or macrophages. In some embodiments, the effector immune cells are T cells expressing CD4 (CD4⁺). In some aspects, TGF-β regulates immunodeficiency virus latency in primary CD4+ T cells in vitro, ex vivo and in vivo.

In embodiments, the T cells have increased expression of CD95 (CD95⁺) over natural or naïve cells. In embodiments, a CD4 T cell population has increased expression of CD95. In embodiments, the immune cells the T cells have decreased expression of T-cell factor 1 (TCF1) over naïve cells.

In some embodiments, the immune cells may be memory immune cells, such as memory T cells. In embodiments, the memory cells are memory CD8+ T cells.

A further aspect of the disclosure is methods for activation of latent immunodeficiency virus in immune cells of a subject having an immunodeficiency virus infection treated with antiretroviral therapy, and contacting the immune cells with a TGF-β inhibitor. In embodiments, the TGF-β inhibitor is Galunisertib. In embodiments, TGF-β inhibitor is contacted with the cells or with the TGF-β molecule while the subject is receiving the antiretroviral therapy.

Another aspect of the disclosure comprises a method for reactivation of HIV in latently infected cells comprising administration of a TGF-β inhibitor. As used herein “activation” and “activated” mean the same as “reactivation” and “reactivated” respectively. Therefore, as would be clear to one skilled in the art, these terms are used interchangeably herein. For example, the disclosure of a latent immunodeficiency virus that can be activated also discloses a latent immunodeficiency virus that can be reactivated as these terms both convey to the skilled artesian an state of transcriptional activity of an immunodeficiency virus as opposed to a state of latency characterized by transcriptional inactivity or silence.

The term “reactivated” or “activated,” as used herein in the context of in vivo reactivated immunodeficiency virus, refers to an immunodeficiency virus that, after a period of latency, becomes transcriptionally active, and in many instances forms infectious viral particles. The term “reactivated” or “activated”, as used herein in the context of in vitro reactivated immunodeficiency virus in a cell, refers to an immunodeficiency virus that, after a period of latency, becomes transcriptionally active. A latent HIV infection may become reactivated pharmacologically or naturally.

HIV can remain latent inside certain cells of the immune system. While in this latent state, the immune system cannot recognize the virus and ART has no effect on the virus. Latency-reversing agents including those disclosed reactivate latent HIV within cells allowing the immune system, and available pharmacological agents to come in contact with the virus. Described herein are latency reversing agents that reactivate latent HIV. In some embodiments these agents include a TGF-β inhibitor. In some embodiments, the TGF-β inhibitor is Galunisertib.

Another aspect of the present disclosure comprises a method for treating a subject infected with HIV, the method comprising reactivation of HIV in latent cells with a TGF-β inhibitor. In some embodiments the TGF-β inhibitor is Galunisertib.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All disclosed methods can be performed in any suitable order unless otherwise indicated or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Blockade of TGF-β Signaling

TGF-β is one of the most potent endogenous immunosuppressive factors and it is considered the master regulator of mucosal immunity(15, 16). TGF-β signaling regulates diverse cellular processes, including proliferation, differentiation, and migration(17), and influences developmental as well as pathological processes such as epithelial to mesenchymal (EMT) transition in fibrosis(18). In the immune system, TGF-β is essential to the maintenance of tolerance(19, 20). Importantly, TGF-β is the most prominent factor responsible for the maintenance of a resting state in CD4+ T cells(21) and loss of TGF-β responsiveness in mature T cells decreases their threshold for activation(22). Moreover, TGF-β also impacts activation status and function of myeloid cells by decreasing antigen presentation capability and maturation and inducing tolerogenic properties(23, 24).

Since the beginning of the HIV-1 epidemic several reports have documented higher concentrations of TGF-β in the blood, lymphoid tissues, and cerebrospinal fluid of people living with HIV-1 (PLWH)(25, 26). Higher levels of TGF-β in PLWH and SIV-infected macaques have been linked to disease progression(25)(27, 28). PBMCs from PLWH spontaneously release high levels of TGF-β(29, 30). Among other mechanisms, TGF-β release particularly in the gut tissue is the result of a biological anti-inflammatory process counteracting HIV-driven microbial translocation and chronic inflammation(31). Paradoxically, this anti-inflammatory, TGF-β-centered response not only exacerbates immunosuppression, but also predisposes for the development of non-AIDS-related, non-communicable disorders(31). Finally, TGF-β-driven fibrosis of lymphoid tissues has been implicated in the CD4+ T cell loss before ART initiation(32) and failure to achieve full immune reconstitution with ART(33).

Importantly, TGF-β inhibits TCR-induced T cell proliferation, CD28-mediated co-stimulation, and activation of T cells (34, 35). Indeed, it has recently been used to promote latency in ex vivo models with primary polarized effector CD4+ T cells (36, 37). Moreover, TGF-β was shown to induce latency in other cell types susceptible to HIV infection(38).

The 3 isoforms of TGF-β (TGF-β1, TGF-β2 and TGF-β3) bind to the specific type I receptor TGFBR1/ALK5 which then undergoes dimerization with the type 2 TGF-βRII. The differential function and signaling mechanism of these major isoforms is still under investigation. However, TGF-β1 appears to be the most prominent regulator of immune responses(39, 40). The TGF-β receptor complex then signals through a canonical pathway by phosphorylating Smad2 and/or Smad3. The Smad complex formed by co-heterodimers with Smad4 interacts with a large variety of co-activators and co-repressor in the nucleus and regulates the transcription of hundreds of genes in a cell type-specific, context dependent way(20).

Galunisertib (LY2157299), a small-molecule selective inhibitor of the TGFBR1/ALK5 (41, 42), has been developed by Eli Lilly as an anti-cancer therapeutic to target the direct and indirect effect of TGF-β on tumor growth. Galunisertib (Gal) progressed to an advanced stage of clinical development (Phase II) and had been tested in several different trials for different solid tumors(42-44), when Eli Lilly decided to end its program to pursue more promising targets(45). TGF-β plays a critical role in maintaining immune cells in a resting state by inhibiting cell activation and proliferation. Resting HIV-1 target cells represent the main cellular reservoir after long-term ART. Given the suppressive activity of TGF-β on different immune cell targets of HIV-1 infection and its role in latency induction(36, 38), it was hypothesized that TGF-β may also exert a suppressive effect on latency reactivation. Moreover, it was hypothesized that if TGF-β inhibited TCR- or PKC-driven HIV reactivation from latency, inhibition of TGF-β signaling may, in turn, increase the frequency of latency reversal following stimulation of these pathways.

These hypotheses were tested using ex vivo models of HIV latency including PBMC from aviremic, ART treated PLWH. Finally, the impact of the TGFBR1 inhibitor galunisertib ex vivo and in SIV infected ART treated macaques was tested. HIV-1 latency models were compared with and without TGF-β and a TGF-β-Type-1 receptor (TGFBR1) inhibitor, galunisertib. The effect of galunisertib (LY2157299) in SIV-infected, ART-treated macaques was tested by monitoring SIV-env expression via PET/CT using the Cu64-anti-gp120 Fab(7D3) probe, along with plasma and tissue viral loads (VL). Exogenous TGF-β reduced HIV-1 reactivation in U1 and ACH2 models. Galunisertib increased HIV-1 latency reversal ex vivo and in PBMC from HIV-1 infected, ART-treated aviremic donors. In vivo, oral galunisertib promoted increased total standardized uptake values (SUVtot) in PET/CT images in gut and lymph nodes of 5 out of 7 aviremic, long-term ART-treated, SIV-infected, macaques. This increase correlated with an increase in SIV-RNA in the gut. Two of the 7 animals also exhibited increases in pVL. Higher anti-SIV T cell responses and antibody titers were detected after galunisertib treatment. In summary, the data suggest that blocking TGF-β signaling simultaneously increases retroviral reactivation events and enhances anti-SIV immune responses. Targeting TGF-β signaling may constitute a novel way to increase viral latency reactivation during ART while potentiating antiviral immune responses.

### Example 1—TGF-β Inhibits PMA-Driven and DC-Driven HIV-1 Latency Reactivation In Vitro

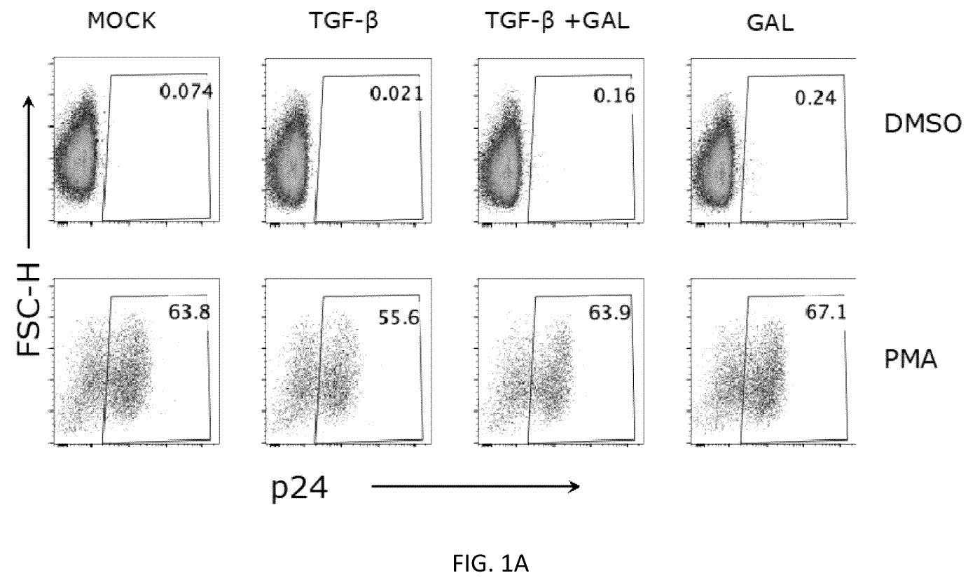

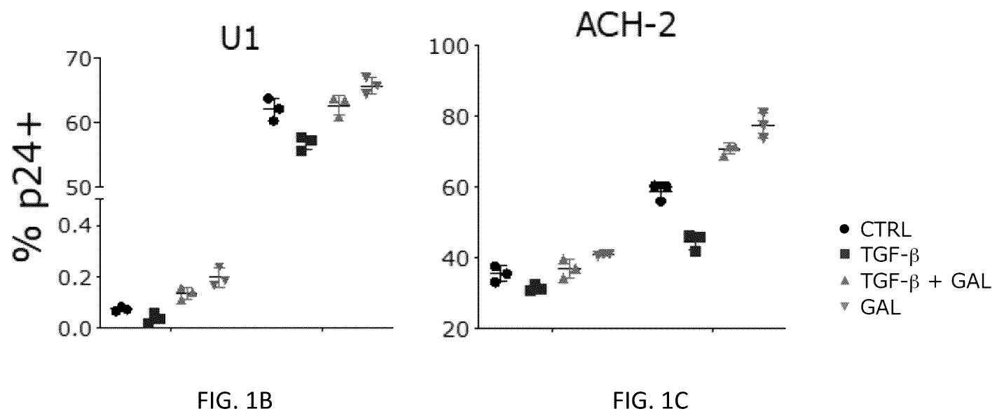

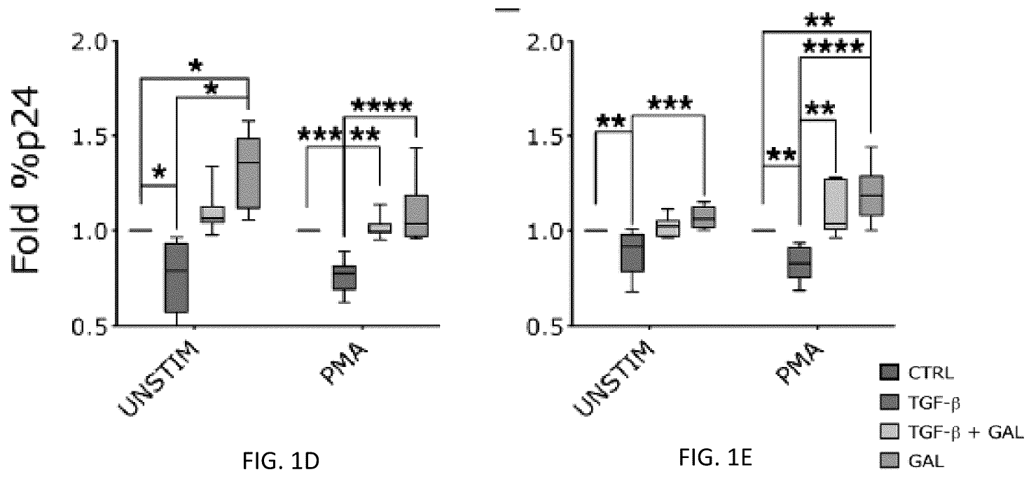

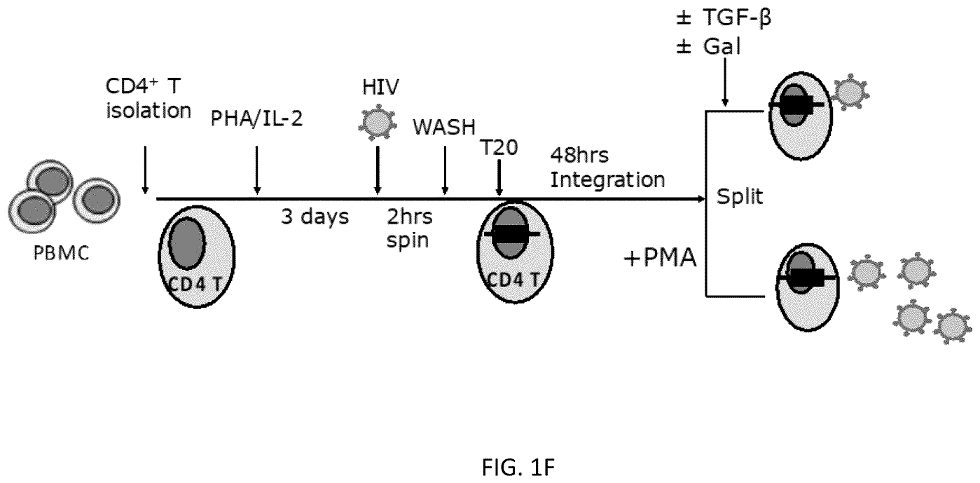

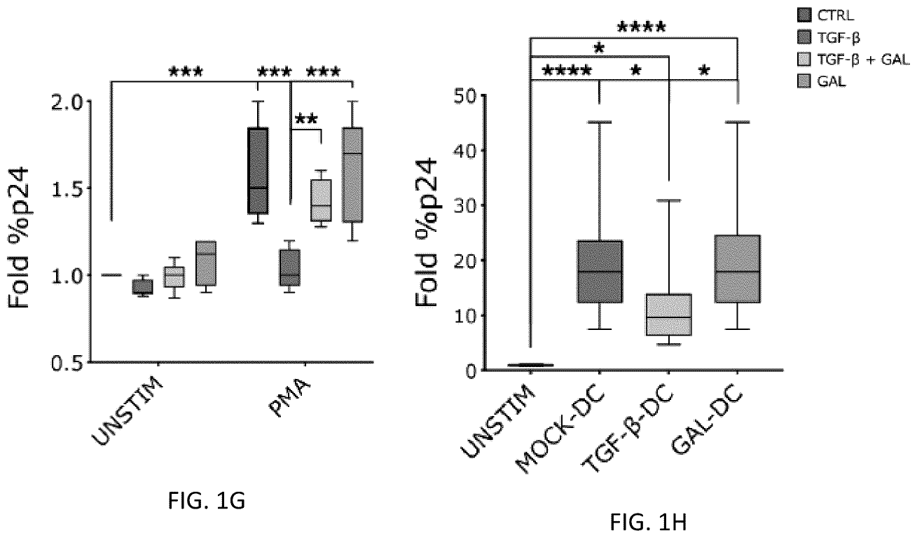

The impact of exogenous TGF-β1 was tested on phorbol 12-myristate 13 acetate-(PMA)-induced HIV reactivation in the U1 and ACH-2 classical cell line models of HIV latency (U1 derived from U937 promonocytic cells (46) and ACH-2 derived from CEM T cells (47)). U1 cells originally contained 2 copies of integrated HIV DNA, although more recently HIV was detected in additional sites (48) and latency is maintained by mutations in the Tat protein (46). The frequency of U1 cells expressing p24-Gag at baseline is very low (FIG. 1A, MOCK-DMSO), but it is readily induced by stimulation with PMA (FIG. 1A MOCK-PMA). Addition of 10 ng/ml of TGF-β1 to the culturing media decreases the frequency of p24-gag+ cells below the baseline level in unstimulated cultures and reduces the PMA driven increase in the frequency of p24-gag+ cells by ˜20% (FIGS. 1A, 1B and 1D). In contrast, blockade of TGF-β1 signaling using galunisertib restores the frequency of p24-gag+ cells in the presence of exogenous TGF-β1 to baseline or PMA-induced levels. Treatment with 1 μM of galunisertib alone appears to slightly increase the frequency of p24-gag+ cells from baseline but has no impact on PMA-stimulated U1 cells (FIG. 1D).

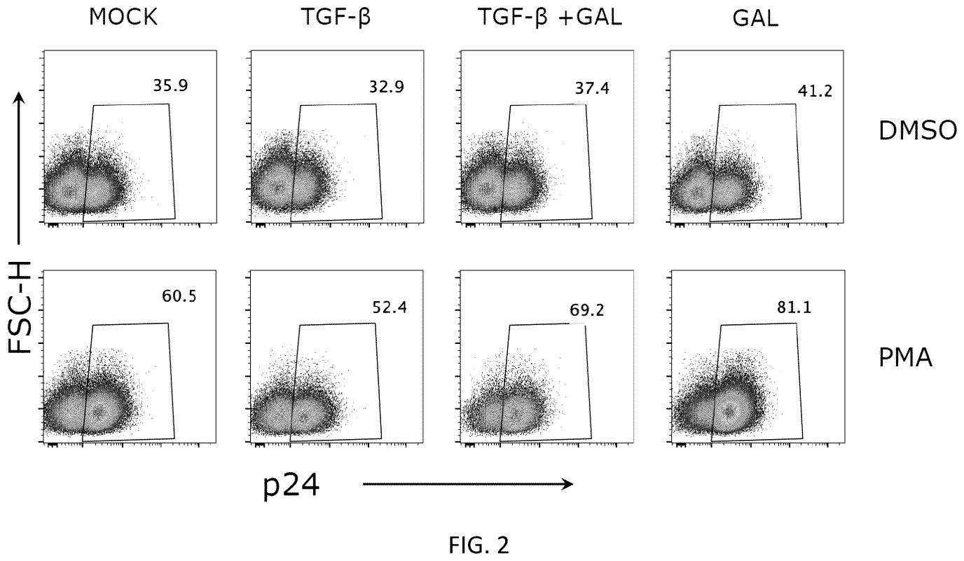

ACH-2 cells were originally reported to carry a single copy of HIV DNA (47), but, as with U1, HIV integration has been detected in multiple sites in more recent culture passages (48). Latency is maintained by a mutation in TAR that impairs the LTR response to Tat (49). In contrast to U1, the frequency of p24-gag+ cells is relatively high in ACH-2 at baseline (FIG. 2, MOCK-DMSO), but increases further in response to PMA (FIG. 2, MOCK-PMA).

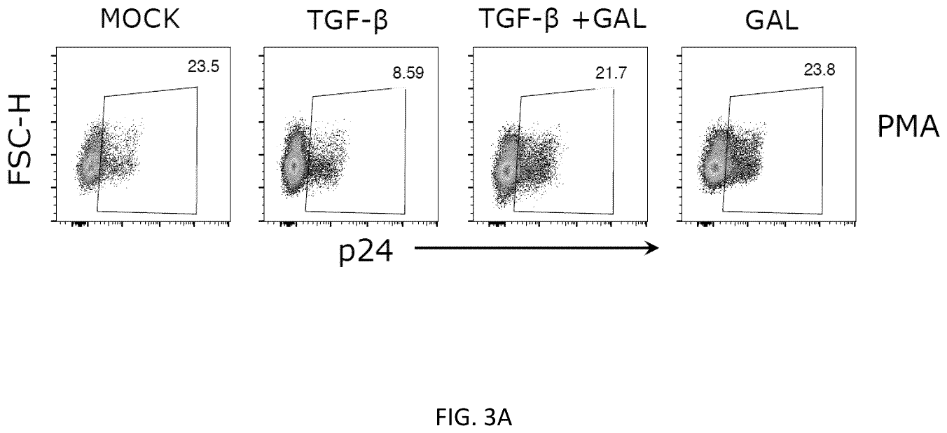

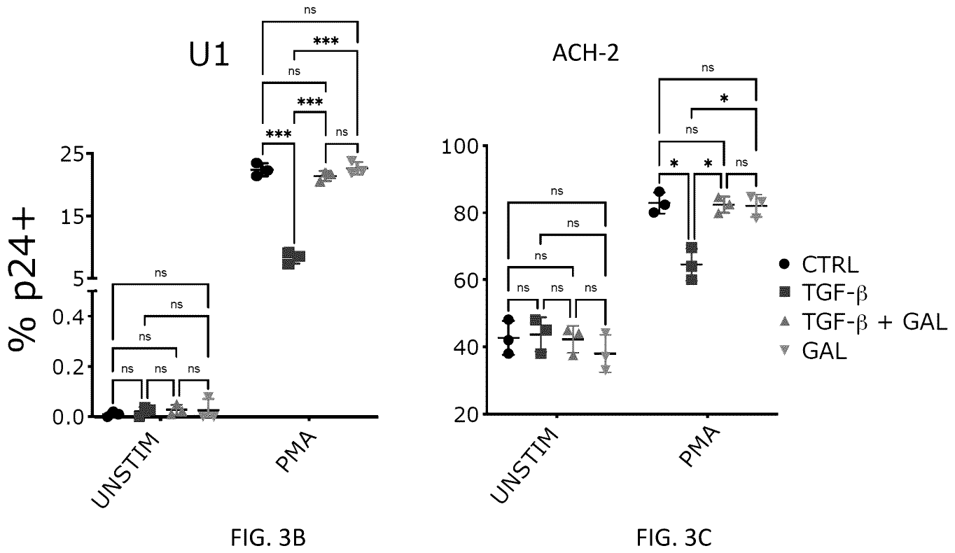

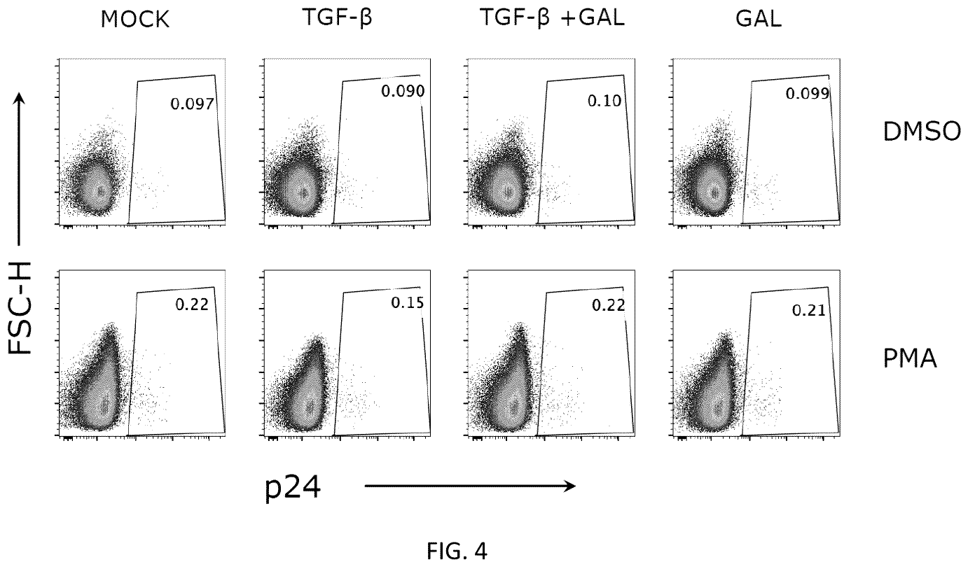

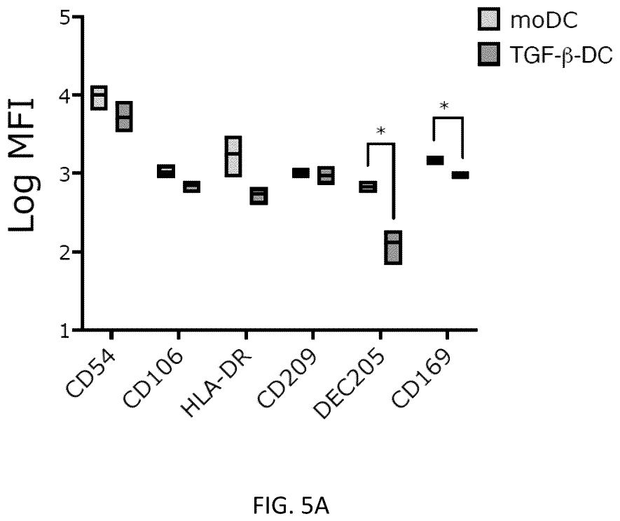

TGF-β1 was able to decrease the frequency of p24-gag+ cells in PMA-stimulated cultures by ˜20% and a smaller but consistent decrease in baseline p24-gag levels was also detected in absence of PMA (FIGS. 1C and 1E). Galunisertib treatment was able to restore frequency of p24-gag+ cells in TGF-1-treated cultures and increase it above PMA-stimulated cultures (FIGS. 1C and 1E) when administered in absence of exogenous TGF-β1. This increase may be attributed to blockade of TGF-β1 in the fetal bovine serum (FBS) used to supplement the culturing media (21). Indeed, in control experiments performed in X-Vivo 15 medium without FBS this Gal-driven increase was absent and the impact of TGF-β1 was more pronounced (FIGS. 3A-3B). However, cell viability in X-Vivo 15 medium was significantly lower than in cultures with FBS for both U1 and ACH-2 cells (U1 an ACH-2 with FBS: Means 98% and 96%, SD 3.4% and 2.4%; U1 an ACH-2 in X-Vivo: Means 68% and 90.1%, SD 12.8% and 3.04%; p<0.001 for both).

Next, the impact of TGF-β1 on a primary model of HIV-1 latency was tested using freshly isolated CD4+ T cells. The experiment was based on the model by Berkhout and colleagues (50) with some modifications (FIG. 1F). Specifically, CD4+ T cells were isolated from HIV-1 uninfected, de-identified donor PBMC and activated them with PHA and IL-2 for 3 days before infection via spinoculation with HIV ADA. After extensive wash of the viral inoculum, cells were cultured 48 hrs in presence of T20 (enfuvirtide) to avoid viral spread beyond the cells with initial integrations. The overall initial infection achieved was very low despite the spinoculation step (˜0.1% of p24-gag+ cells, FIGS. 3A-3B). However, PMA stimulation was able to increase the frequency of p24-gag+ cells consistently (FIG. 1G and FIG. 4) and this was significantly inhibited when TGF-1 (10 ng/ml) was added concomitantly with PMA. In contrast the addition of both TGF-β1 and galunisertib (1 μM) or galunisertib alone did not alter the frequency of p24-gag+ cells from the level in control cultures (FIG. 1G and FIG. 4).

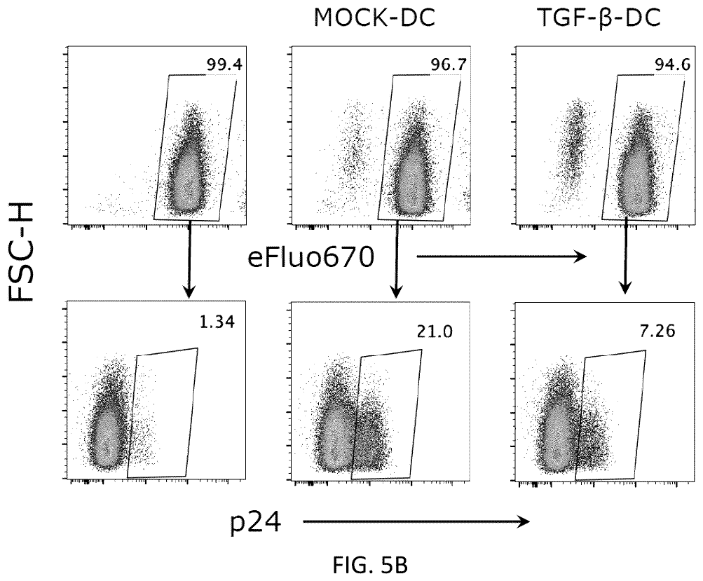

Finally, considering that dendritic cells (DC) are important players in reversing HIV latency (51) and that TGF-β1 impairs DC activation (52, 53), the impact of TGF-β1 on DC-mediated HIV-1 latency reactivation (50, 54) was tested. Monocyte derived (mo)DCs were generated from primary monocytes as previously described (55) and treated or not with TGF-β1 for the last 2 days of culture. moDCs generated in the presence of TGF-β1 exhibited a less mature phenotype with tendency toward lower HLA-DR and significantly lower DEC-205 expression (FIG. 5A). When co-cultured with eFluo670 pre-labeled U1 cells, moDCs were able to promptly activate HIV-1 expression with the frequency of p24-Gag+ cells increasing of several folds within a few hours (FIG. 1H and FIG. 4). However, the frequency of p24-gag+ cells in the cocultures with TGF-β1-treated DCs was significantly less than in co-cultures with untreated DCs and this was blocked when TGF-β1-treatment of DCs occurred in presence of galunisertib (FIG. 1H and FIG. 5).

### Example 2—Blocking TGF-β Signaling Increase LRA-Induced HIV-1 Reactivation Ex Vivo

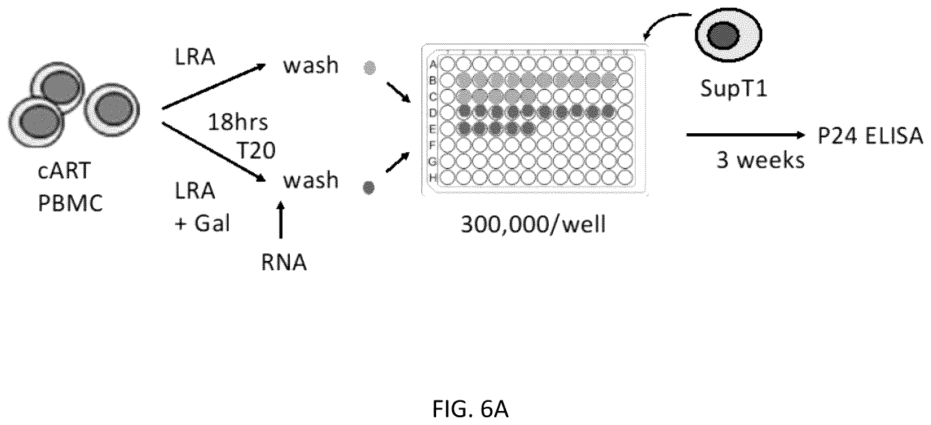

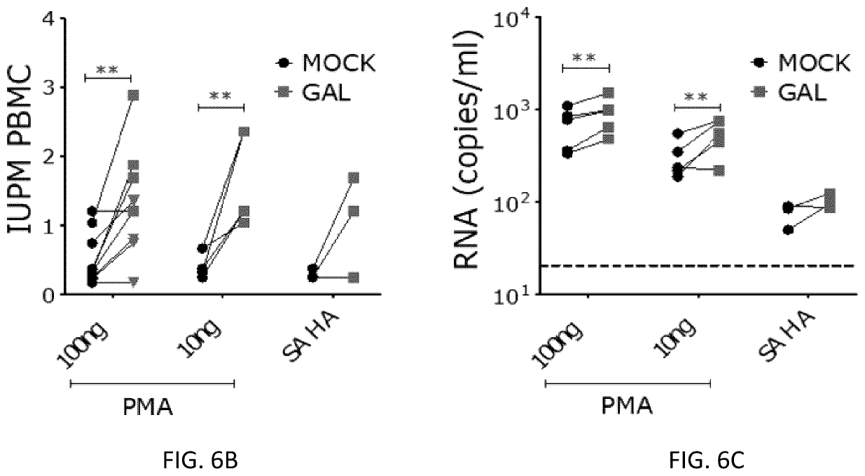

Considering that TGF-β is present in FBS, used to supplement PBMC and T cell cultures, and that PBMC from people living with HIV-1 release TGF-β in culture supernatants (56-58), we hypothesized that blocking TGF-β while reversing HIV-1 latency ex vivo from PBMC of ART-treated, aviremic individuals may lead to higher frequency of HIV-1 reactivation than in untreated cultures. To test this hypothesis, we used a modified version of the classical quantitative viral outgrowth assay (qVOA) (59). We used whole PBMC to assess potential latency reactivation from any HIV-1-infected circulating cell and not only from resting CD4+ T cells (FIG. 6A). PBMC from two different sets of aviremic, ART-treated donors (Table 1) were activated with PMA (at 100 ng/ml or 10 ng/ml) and with Vorinostat (SAHA, at 1 μM) in presence of T20 and galunisertib (1 μM) or mock solution. After overnight incubation supernatants were collected and assayed for both vRNA in a subset of donors (n=5) and viral outgrowth (n=9). After washing out the PMA, cells were plated at 3×10⁵/well in 96 well plates with or without galunisertib in replicates (>14 replicate wells) and SupT1 cells were added to amplify the infection. Infectious unit per million cells (IUPM) were calculated based on the number of infected wells as determined by p24 ELISA (FIG. 6A). A significantly higher frequency of latency reactivation events occurred in presence of galunisertib in PBMC from most donors (FIG. 6B). This occurred with both high and low PMA concentrations and when reactivation was performed with Vorinostat. No other latency reversal agents (LRA) were tested. Moreover, higher levels of vRNA were detected after PMA stimulation in galunisertib-treated PBMC than in mock treated (FIG. 6C).

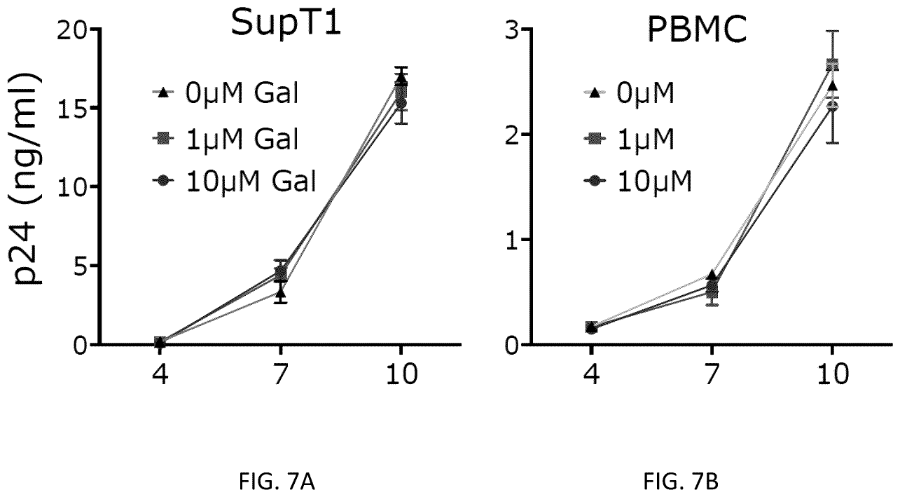

To control that addition of galunisertib would not directly impact HIV-1 replication in SupT1 cells or PBMC increasing the chances to detect infection, ex vivo infection of SupT1 cells and PBMC from healthy donors in presence of different concentration of galunisertib was performed (FIG. 7). Galunisertib had no impact on the HIV-1 growth curves in these in vitro infections.

TABLE 1
Participant's characteristics

| | | | | | | | Time |
|---|---|---|---|---|---|---|---|
| | | | | | | | on |
| | | Race and | | CD4 | VL | | ART |
| ID | Sex | ethnicity | Age | (counts) | (copies/mL) | ARVs | (y) |
| ART1 | M | Black (AA) | 55 | 630 | <40 | RIL/TAF/FTC | 20 |
| ART2 | M | White not | 60 | 392 | <40 | DTG/FTC/TAF | 24 |
| | | Hispanic | | | | | |
| ART3 | M | With note | 57 | 586 | <40 | ABV/DTG/3TC | 20 |
| | | Hispanic | | | | | |
| ART4 | F | Black (AA) | 56 | 949 | <40 | EFV/3TC/TDF | >20 |
| ART5 | M | White not | 56 | 551 | <40 | EVG/Cob/TAF/FTC | 13 |
| | | Hispanic | | | | | |
| RAD1 | M | Black (AA) | 25 | NC | <40 | TDF/FTC/RIV/ATZ | 5.32 |
| RAD2 | M | Hispanic/Latinx | 24 | NC | <40 | ELV/Cob/TDF/FTC | 3.18 |
| RAD3 | M | Black (AA) | 23 | NC | <40 | EFV/TDF/FTC | 4.13 |
| RAD4 | M | Black (AA) | 32 | NC | <40 | TDF/FTC | 4.77 |

Abbreviations in Table 1: VL, viral load; ARVs, antiretroviral therapies; AA, African American; RIL, rilpivirine; TAF, tenofovir alafenamide; FTC, emtricitabine; DTG, dolutegravir; ABV, abacavir; 3TC, lamivudine; TDF, tenofovir; EVG, elvitegravir; Cob, cobicistat; NC, not counted; RIV, ritonavir; ATZ, azidothymidine; ELV, elvitegravir; EFV, efavirenz.

### Example 3—Blocking TGF-β Leads to SIV Reactivation In Vivo

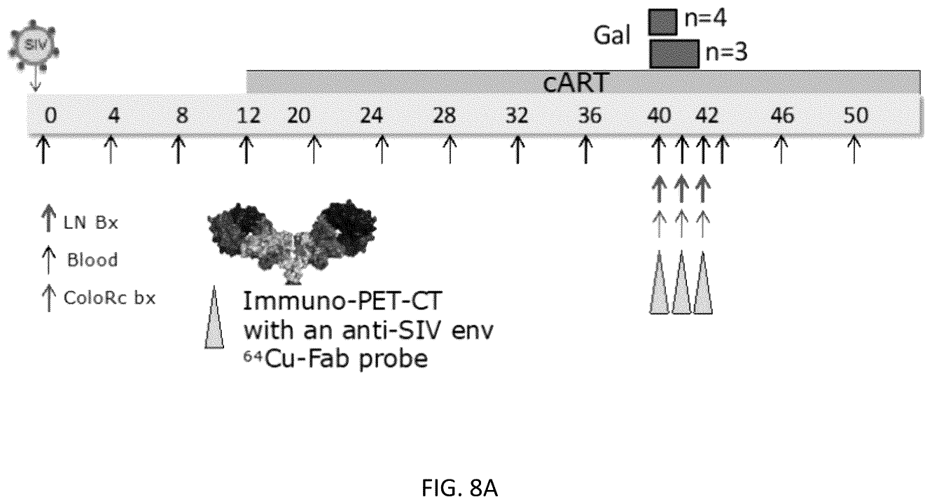

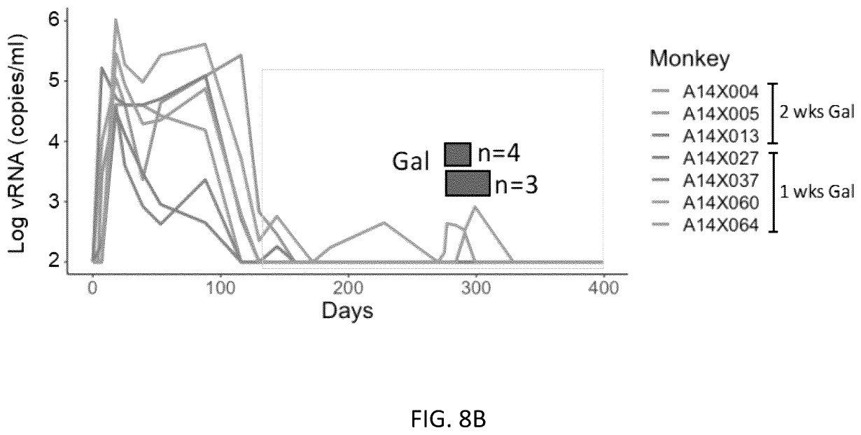

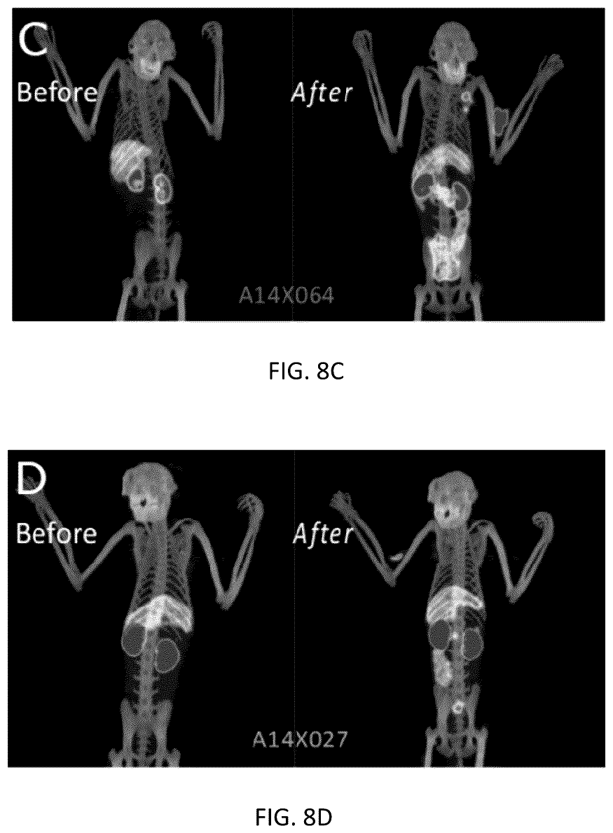

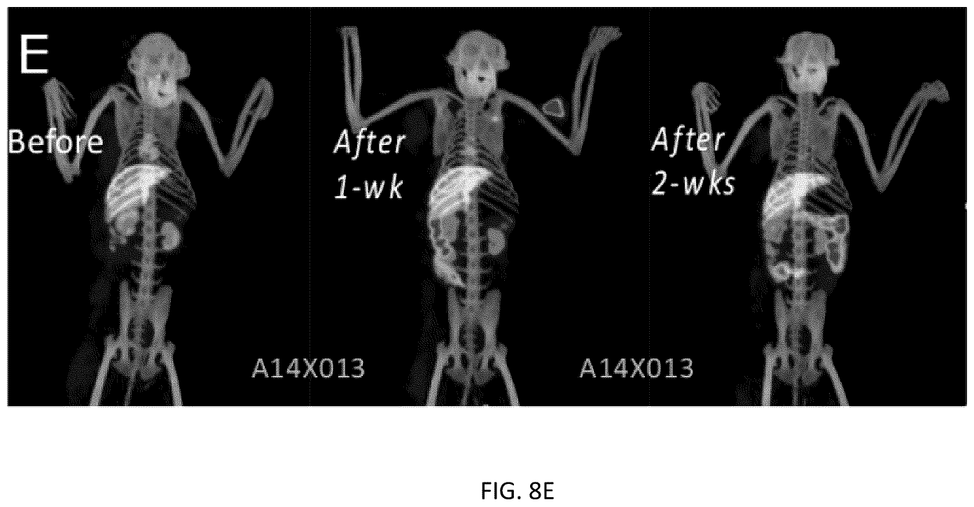

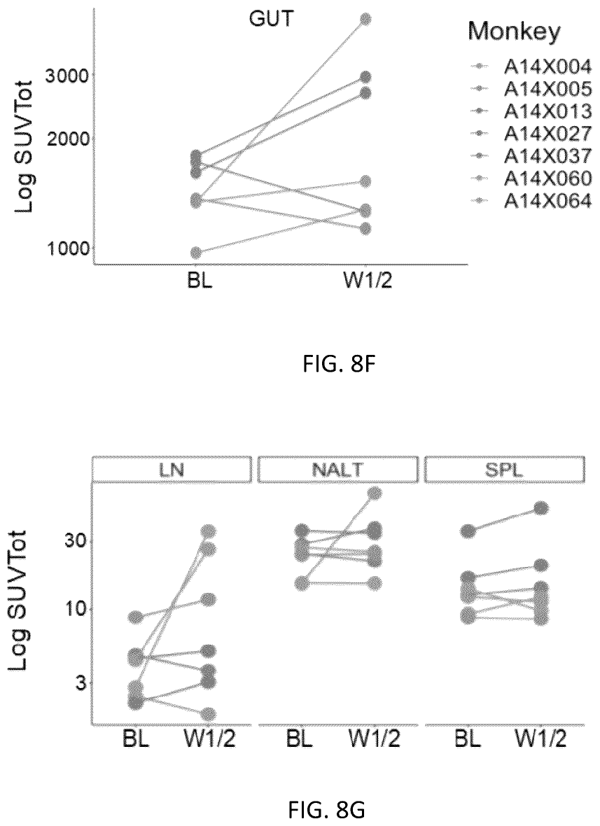

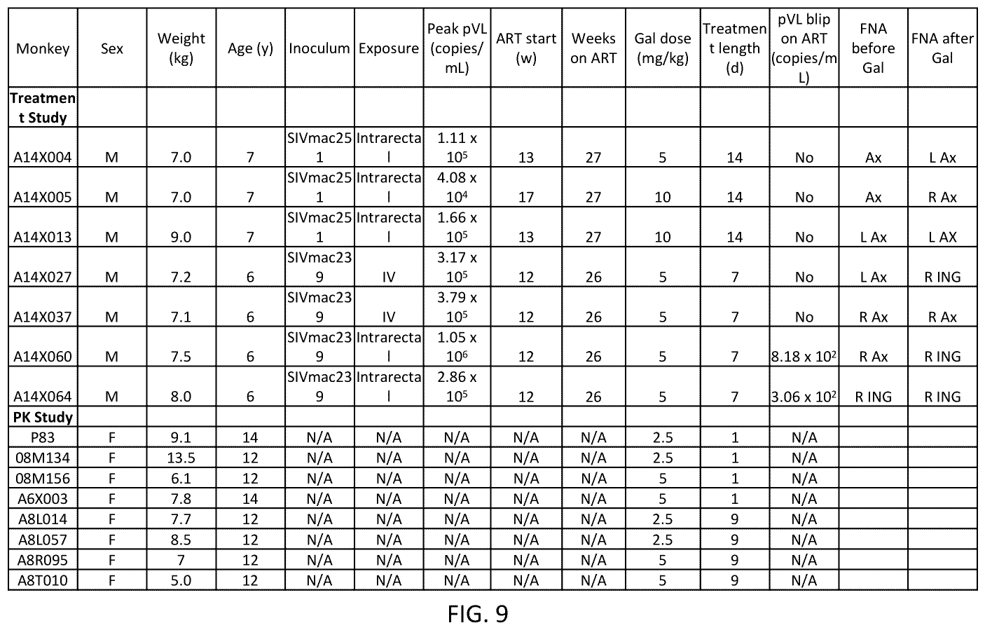

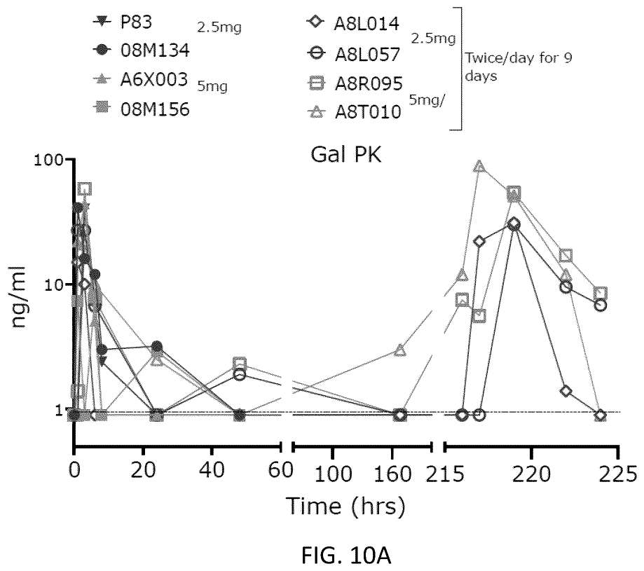

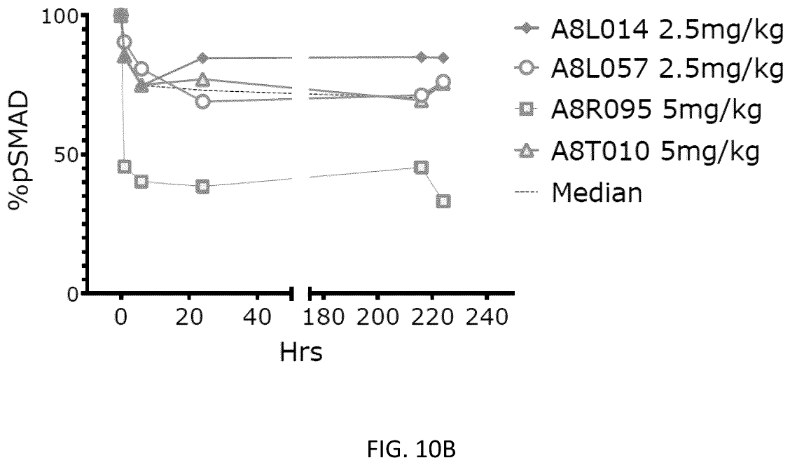

To test whether galunisertib might facilitate spontaneous episodes of HIV latency reversal, especially in tissues rich in TGF-β such as the gut, we administered galunisertib at 5 mg/kg or 10 mg/kg via oral gavage to 7 SIV infected, ART-treated macaques (FIG. 8A and FIG. 9). Macaques were infected intrarectally (2000TCID50), or intravenously (IV) (300TCID50) of SIVmac251 or SIVmac239 (FIG. 9). All macaques but one were ARV suppressed (plasma viral load; pVL<100 copies/ml) for at least 100 days before galunisertib treatment. Galunisertib was used at 5-10 mg/kg, which is equivalent to the dose administered in clinical trials for cancer treatment (43, 60) and predicted to be within the therapeutic window in humans by extensive PK/PD studies (42, 61). PK in rhesus macaques was evaluated in a separate group of naïve animals (FIG. 9) and found to be substantially lower than in humans (FIG. 10A and (61)). However, examination of pSMAD2/3 over total SMAD in PBMC suggested that galunisertib was active in the macaques at these concentrations (˜25% reduction in pSMAD2/3 FIG. 10B).

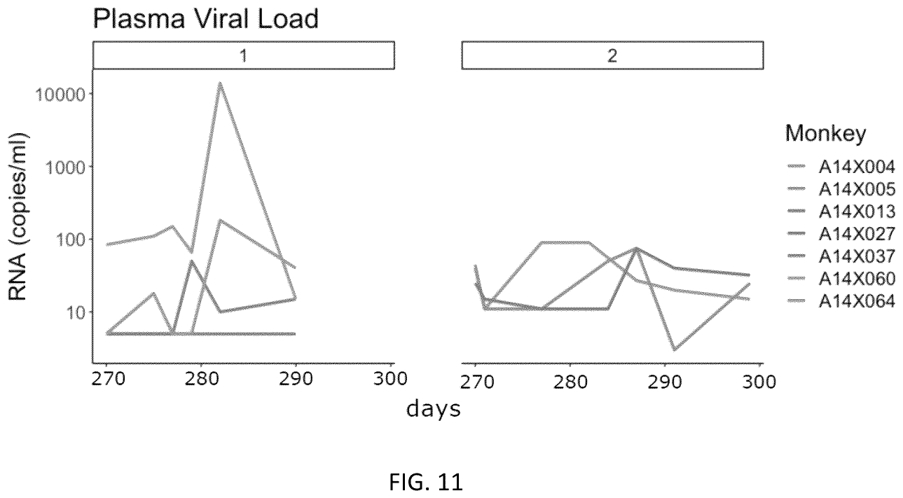

Galunisertib was administered orally twice per day for 1 week for 4 macaques and 2 weeks for 3 macaques. An increase in pVL over 100 copies/ml was detected in 2 of the 7 macaques (FIG. 8B and FIG. 9). Additional small changes in pVL occurred below 100 copies/ml in the other macaques when pVL was analyzed by ultrasensitive (LLOD 5 copies/ml) assay at Leidos. However, most of the macaques had detectable pVL at baseline when using the ultrasensitive assay and extensive baseline analysis was not performed to determine whether these changes represented a true deviation from baseline (FIG. 11).

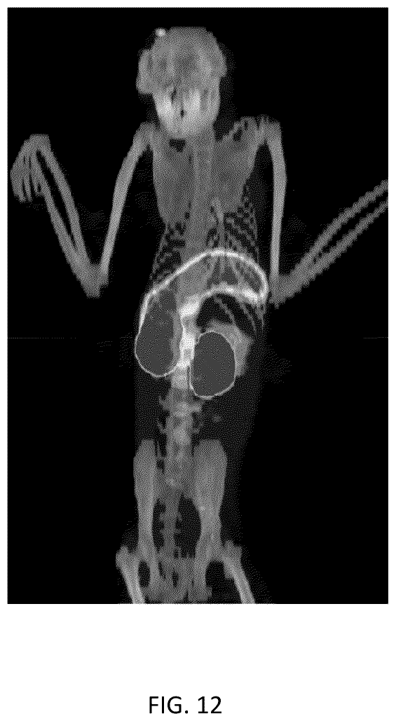

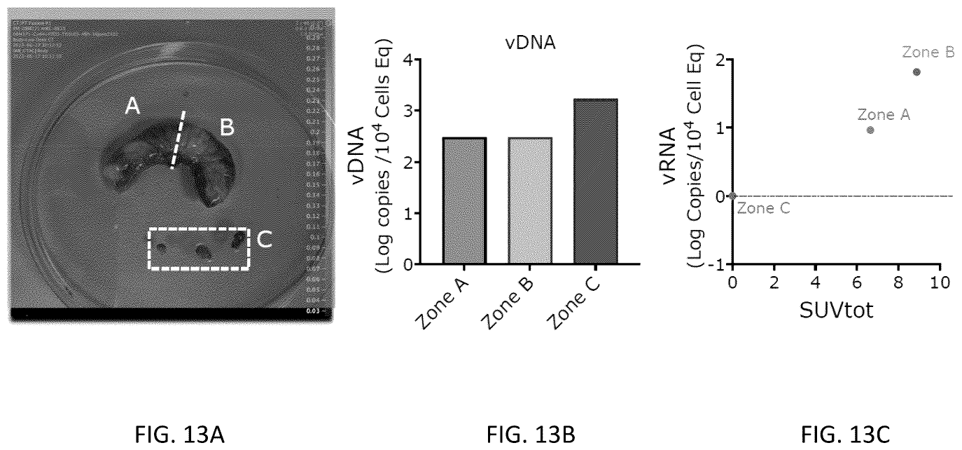

ImmunoPET/CT images with the validated ⁶⁴Cu-DOTA-F(ab′)2 p7D3 anti SIV-env probe (62, 63), colorectal biopsies (bx), lymph node (LN) fine needle aspirates (FNA), and blood samples were collected at baseline (day −1 of treatment) and on day 7 or 14 of treatment. 223 Notably, a clear extensive viral reactivation occurred in the gut of 3 of the 7 macaques as documented by the notable increase in PET signal (A14X064, A14X027 and A14X013, FIGS. 8C-8D-8E). A smaller increase in the gut was also present in A14X004 and A14X060 (FIG. 8F). An increase in PET signal also occurred in the LNs of A14X064, A14X013 and A14X060 (FIGS. 8C and 8E). These increases were captured by the change in total standard uptake values (SUV) in the different anatomical areas as summarized in FIGS. 8F and 8G. No reactivation was detected in macaque A14X037 and A14X005. The ⁶⁴Cu-DOTA-F(ab′)2 p7D3 was previously validated in SIV uninfected macaques in (62). However, a probe generated using a rhesus IgG1 Fab against an irrelevant antigen ⁶⁴Cu-DOTA-F(ab′)2 plgG1 in an SIV infected macaque was used as further control (FIG. 12). Moreover, a resection of the PET positive area in the small intestine of an SIV infected macaque was used as control of the association between the PET signal and the level of SIV replication in a specific tissue (FIG. 13).

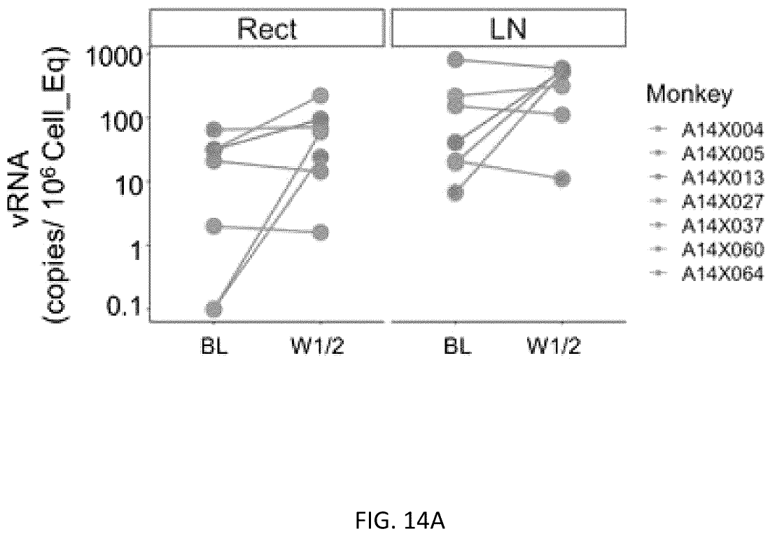

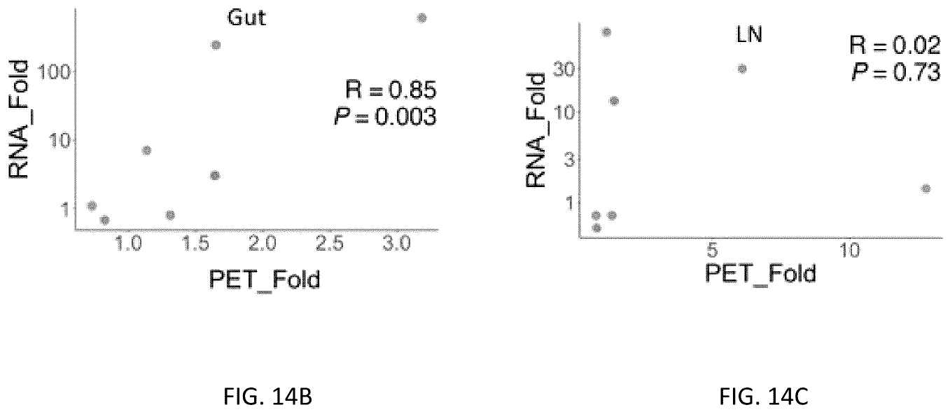

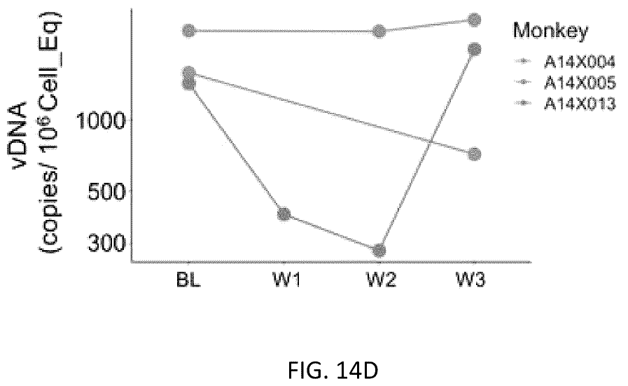

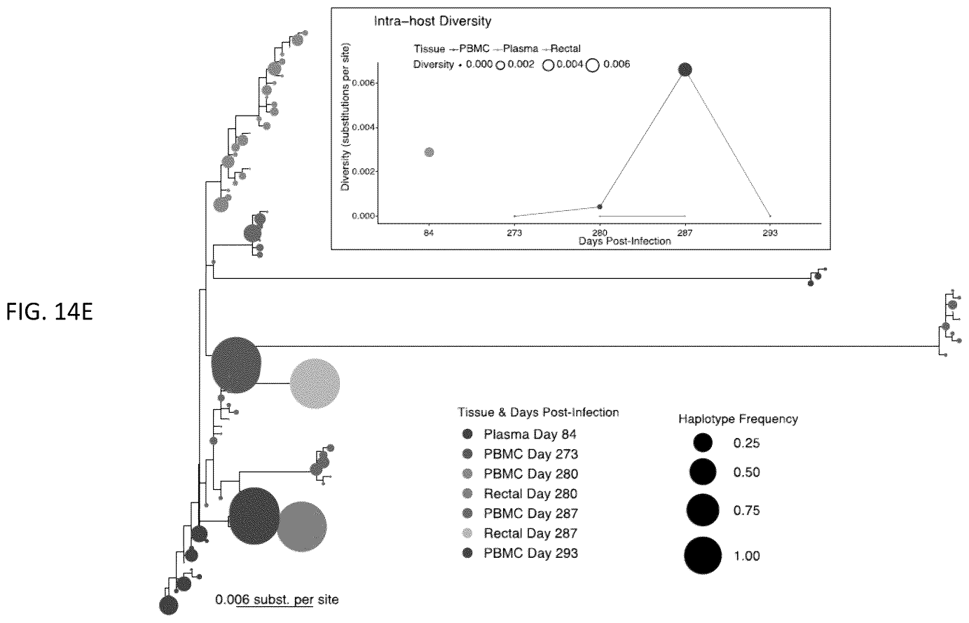

Viral reactivation in the macaques was confirmed by large (1 to 3 log) increases in cell associated vRNA (CA-RNA) observed when colorectal biopsies and LN FNA were assayed for RNA (FIG. 14A). Notably, the increase in rectal biopsy CA-RNA correlated well (R=0.85; FIG. 14B; Upper) with the increase in SUVtot in the gut region. This occurred even though biopsies were collected from the colorectal area while the SUVtot for the gut represent the signal in the entire intestine. In contrast, no correlation was present between vRNA in the FNA that sampled a single lymph node (FIG. 9) and the SUVtot signal in both axillary LN (FIG. 14B; Lower).

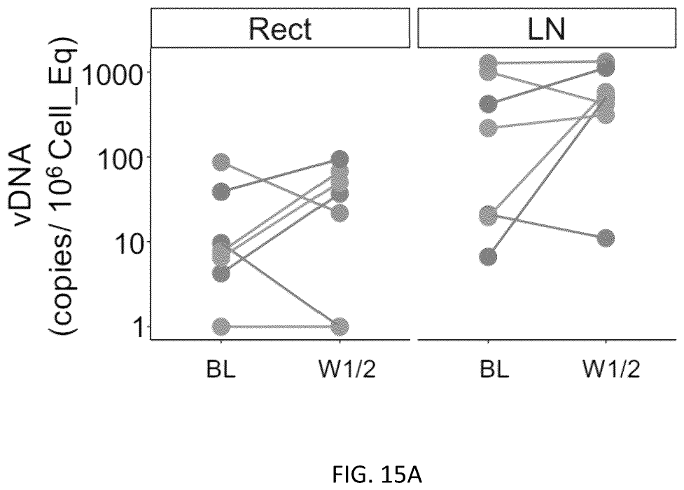

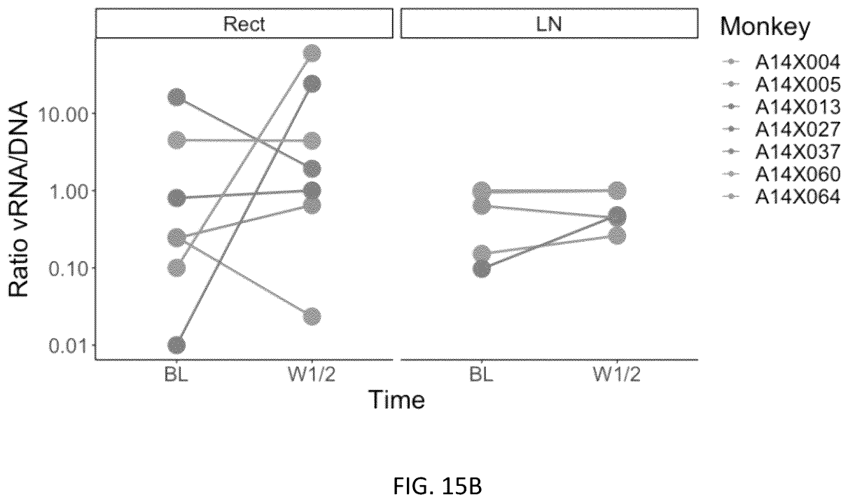

In some macaques vRNA increase was paralleled by a small increase in CA-DNA (FIG. 15A, e.g., in A14X060 for colorectal bx and A14X027 and A14X064 for LN). In other macaques CA-RNA increase was not accompanied by an increase in CA-DNA and CA-DNA varied substantially between baseline and post-galunisertib treatment. However, the ratio CA-RNA/DNA increased substantially in at least 2 macaques (A14X027 and A14X064, FIG. 15B).

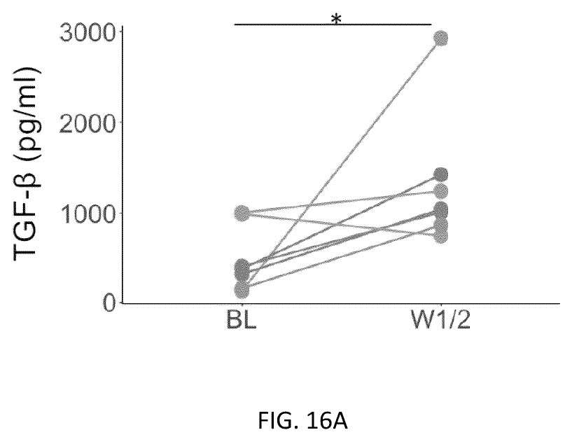

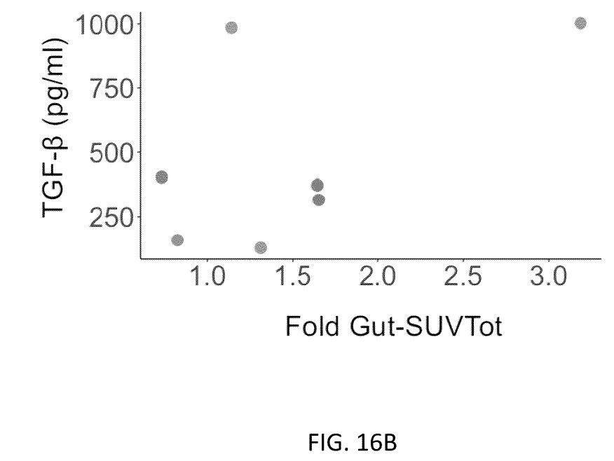

The levels of CA-vDNA in the PBMC of the 3 macaques that were treated for 2 weeks (samples were not available for the animals treated for 1 week) were examined. Interestingly, we noted a drop in the CA-vDNA levels in the PBMC of A14X013. Since this macaque had a strong viral reactivation signal via PET (FIG. 8E), we considered this drop interesting and worth further evaluation by viral sequencing. The increase in CA-vRNA in the tissues of this macaque was balanced by an increase in CA-DNA, suggesting that galunisertib may have induced recirculation of infected cells from blood to tissues and vice versa. This was supported by the sequencing data that demonstrated a substantial increase in the diversification of the intra-host viral quasispecies obtained from A14X013 PBMCs during the two weeks of galunisertib treatment (FIG. 14D). The inferred phylogeny of the intra-host quasispecies at the different time points and anatomical locations suggest the expansion of multiple viral lineages at week 1 and 2 after galunisertib initiation in PBMC (FIG. 14D). Viral diversity was low at the time of galunisertib initiation in PBMC. This was followed by the high levels of diversification in blood while observing compartmentalization between PBMC and rectal tissues that additionally displayed low diversity populations. At week 3 after galunisertib treatment initiation (1 week after the end of the treatment), viral diversity in blood dropped and the new dominant viral populations in blood were closely related to the later viral populations in rectal tissues detected a week earlier. Overall, this suggests a significant viral expansion during galunisertib treatment of multiple infection foci and migration of viral populations between tissue and blood in A14X013. Interestingly, we observed an increases in the levels of TGF-β in blood after galunisertib treatment (FIG. 16A). However, reactivation was not linked to baseline concentrations of circulating total TGF-β (FIG. 16B).

### Example 4—Blocking TGF-β Enhances SIV-Specific Responses

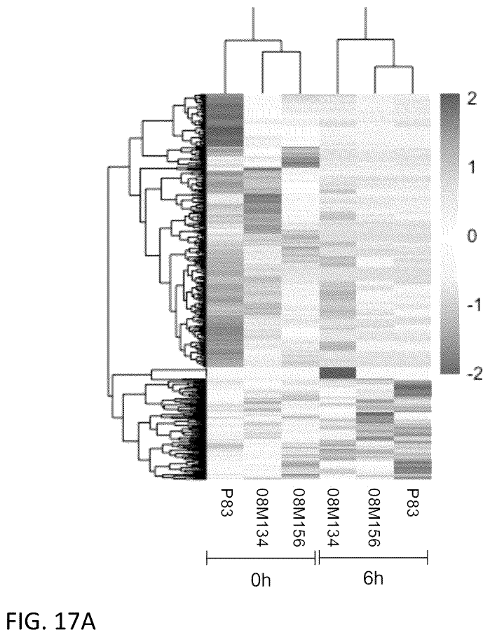

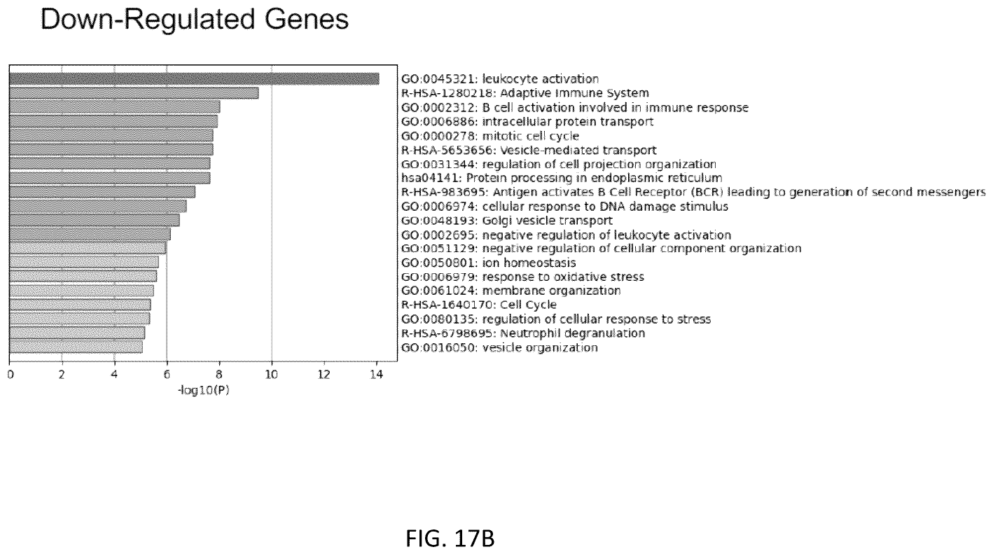

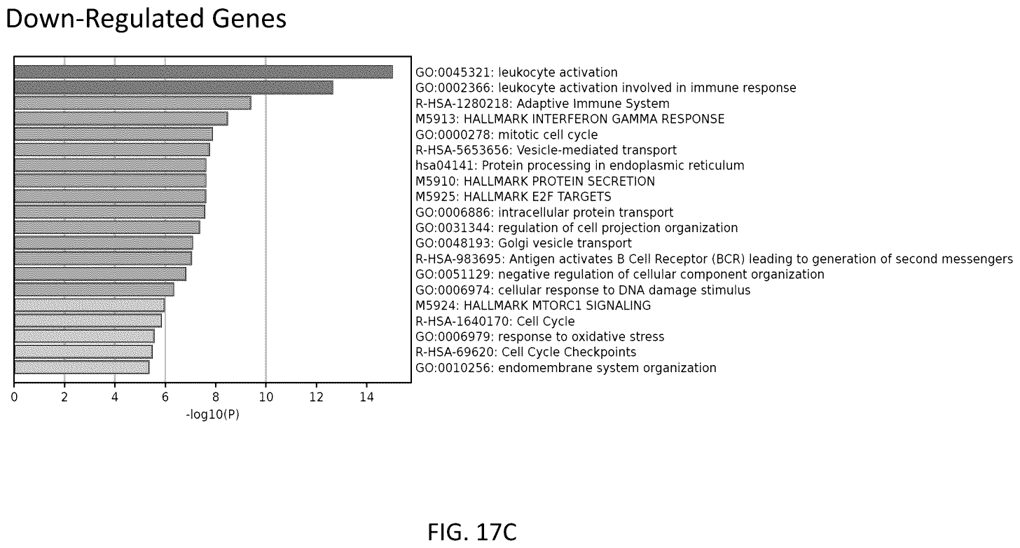

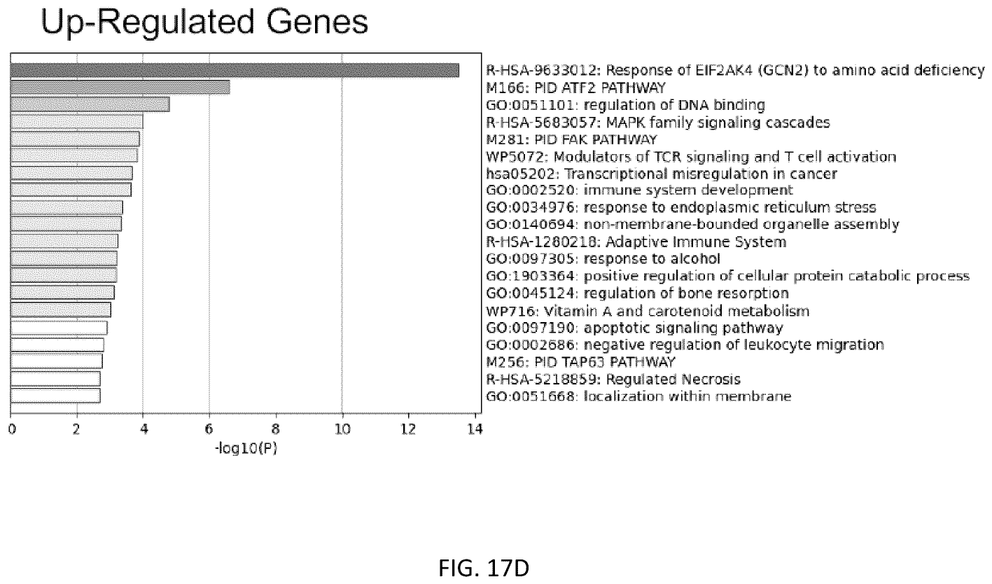

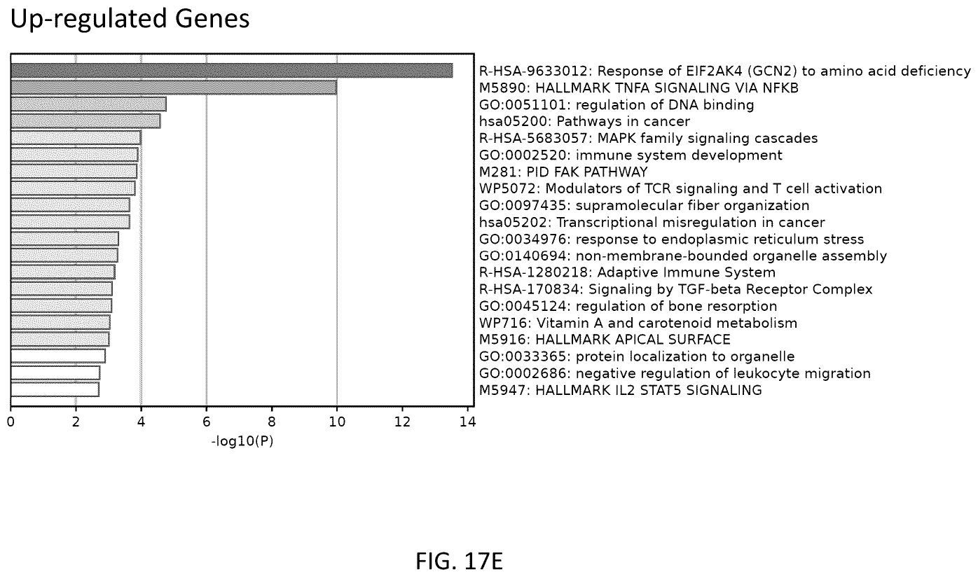

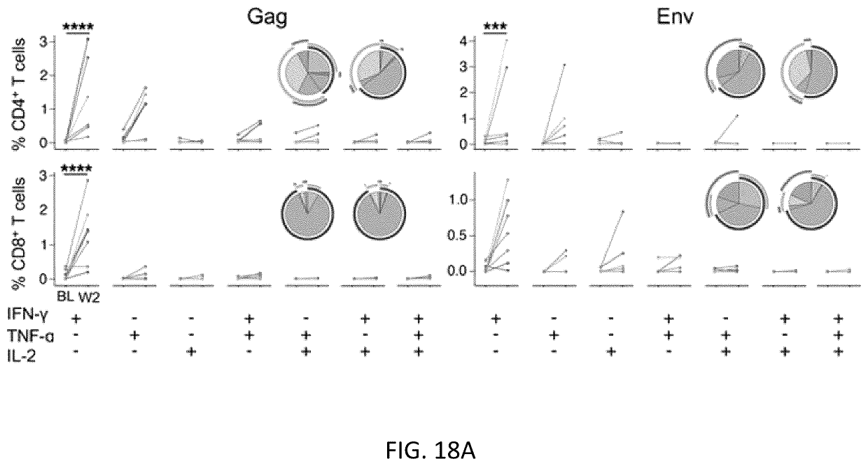

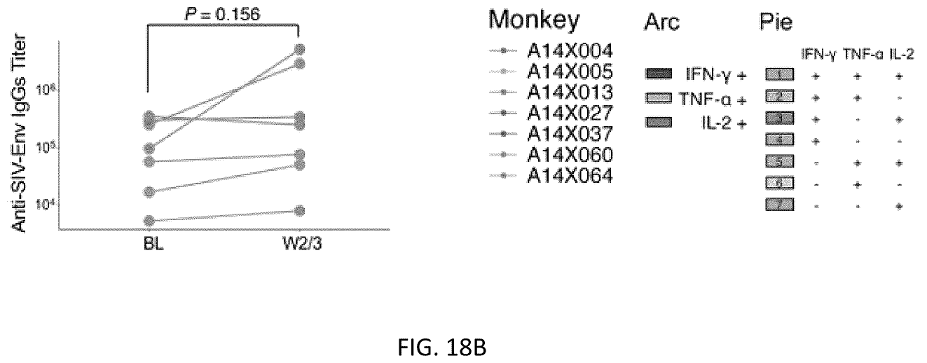

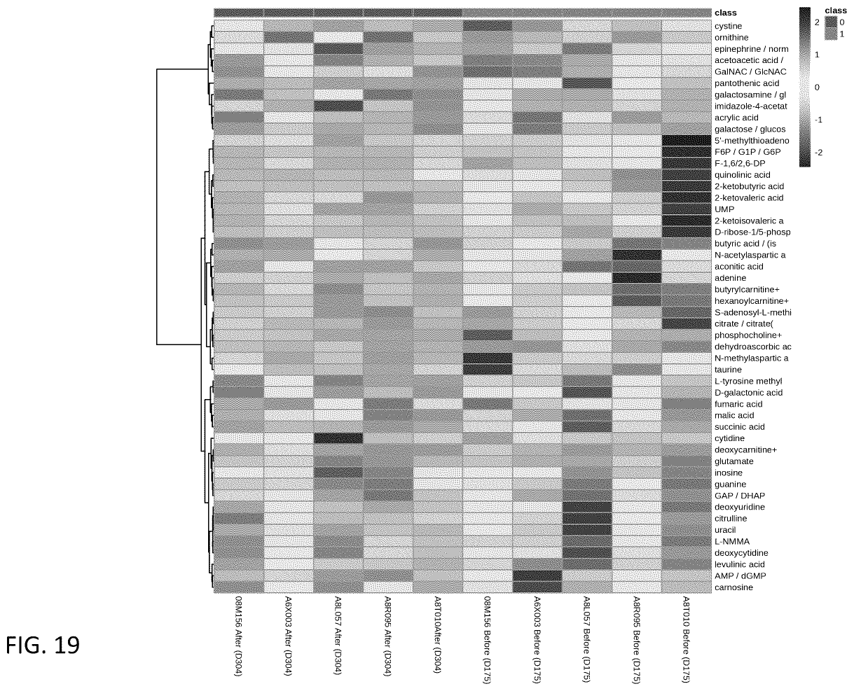

Since TGF-β is primarily an immunosuppressive factor, blocking TGF-β1 signaling was expected to impact phenotype and function of immune cells independent of SIV infection. Indeed, a preliminary mRNAseq analysis of CD4 T cells from before and after (6 hrs) Galunisertib treatment in 3 naïve macaques suggested several changes in pathways regulating immune functions such as leukocyte activation and adaptive immune system (downregulated) and regulation of DNA binding, MAPK, and FAK pathways and TCR signaling (upregulated) (FIGS. 17A-17E). Hence, the impact of TGF-β1 blockade with galunisertib on SIV-specific T cell and B cell responses was investigated. T cell responses were probed via classical PBMC stimulation with gag and env peptide antigen followed by detection of intracellular IFN-γ, TNF-α, and IL-2 by flow cytometry. significant increase in the frequency of IFN-γ producing CD4 and CD8 T cells was found following both gag and env peptides stimulation (FIG. 18A). However, no increase in the frequency of cells producing multiple cytokines (pluripotent cells) was observed Subset analysis using a CD95 memory marker was not performed. Finally, in 3 of the 7 macaques there was a notable (0.5 to 1 Log) increase in the titer of SIV-env specific antibodies following galunisertib treatment (FIG. 18B). However, this increase did not occur in macaques with large viral reactivation as measured by TotSUV increase. Rather antibody titers increased in animals with no change (A14X037) or relatively lower increase (A14X027 and A14X004) in gut TotSUV (FIG. 8F).

### Example 5—Targeting TGF-β Leads to Attrition of the Viral Reservoir During ART

Seven rhesus macaques were infected intravenously with the barcoded SIVmac239M2 and treated with ART from week 6 post-infection (pi). After 29 weeks on ART, the macaques were treated with a more extensive Gal regiment consisting of four 2-weeks cycles of oral Gal (20 mg/kg) twice per day. Blood, lymph nodes and mucosal samples were collected before and after each of the first 3 treatment cycles and at the end of the 4th cycle. PET/CT imaging using the ⁶⁴Cu-DOTA-F(ab)2 p7D3 probe were performed before and after the first 3 Gal cycles. ART was interrupted 3 weeks after the end of the 4th cycle and the macaques euthanized 7 weeks later.

Gal treatment induced viral reactivation in blood with blips above 100 copies/ml in 5 out of the 7 macaques. Extensive SIV reactivation in tissues was documented by PET/CT in all macaques particularly after cycle 2. Immunophenotyping and barcode analysis is undergoing. However, preliminary data suggest that Gal stimulated an effector phenotype in T cells with increased expression of CD95 and shedding of CD62L. However, Gal did non induce systemic immune activation at least at the end of the 1st cycle, as suggested by PBMC RNAseq data and lack of increased expression of CD69 and CD38 on T cells. Finally, cell-associated viral DNA (vDNA) decreased by over a Log in lymph nodes and colorectal biopsies of all treated macaques by the end of Gal cycle 4th compared to before Gal. This suggests that targeting TGF-β leads to attrition of the viral reservoir during ART, particularly in tissues, by a combination of an increased frequency of SIV reactivation events and enhanced SIV-specific responses.

### Discussion

HIV-1 persists in different cell types during ART and it is now clear that the mechanisms of viral persistence are heterogeneous. HIV-1 transcriptional activity varies in different cell types and is dependent, at least in part, on tissue location and environmental cues. HIV-1 latency in T cells is maintained through diverse mechanisms that include blocks in transcriptional elongation, completion, and splicing(2). The differential role of these blocks on HIV-1 expression in T cells depends on the tissue and microenvironment where infected cells reside and the same factors may also influence the ability of HIV-1 to emerge from latency (64, 65). Similar heterogeneous, tissue/environment-dependent mechanisms may be at play in regulating HIV-1 replication in other HIV-1 cell reservoir types such as myeloid cells. Finally, the long-term persistence of these infected cells may be dependent on homeostatic(66, 67) or antigen-driven proliferation(3, 68) with a potential contribution of promoter insertion mutagenesis(69, 70). The different cellular processes that regulate the dynamics of HIV-1 reservoir remain to be fully clarified. However, a common characteristic shared by HIV-1 target cells of both T and myeloid cell lineages that constitute the HIV-1 “invisible” reservoir is their “resting” phenotype. TGF-β represents a common factor released at high level in PLWH. It limits the ability of HIV-1 infected cells to respond to activation stimuli and raises the threshold for TCR-driven proliferation(71), while not fully impairing homeostatic proliferation(72). The capacity of TGF-β to broadly inhibit cell activation in several different potential HIV-1 cellular reservoirs, while not fully blocking the ability of infected cells to proliferate led us to test the hypothesis that TGF-β might be a major factor that contributes to the low inducibility of intact proviral sequences in a wide range of cell types(13). We reasoned that inhibiting TGF-β signaling might decrease the threshold for transcription and translation of HIV-1 proteins in cells activated by different stimuli both ex vivo (in presence of exogenous TGF-β1, including the TGF-β1 present in FBS(21)) and in vivo. In tissues, especially mucosal tissues such as the gut, TGF-β is a major player and regulator of tissue homeostasis(73). Moreover, the gut is a site of constant antigenic exposure. This is especially true in HIV infected subjects where compromised gut barrier function is a source of inflammatory stimuli that likely drives stochastic HIV-1 reactivation. Indeed, in vitro, using the classical cell line models ACH-2 and U1, we demonstrated that addition of exogenous TGF-β decreases the ability of PMA, a powerful PKC activator, to induce HIV-1 reactivation and, as consequence, fewer cells expressed HIV antigens in presence of TGF-β. This effect was blunted when TGF-β was added in presence of galunisertib. In some cases, we detected enhanced HIV-1 reactivation in the presence of galunisertib alone, which likely reflected galunisertib-mediated inhibition of FBS-derived TGF-β.

Here, TGF-β has been demonstrated to act both directly and indirectly on HIV-1 infected cells. Indeed, TGF-β-treated moDCs exhibited a reduced ability to reactivate HIV-1 from latently infected T cells. Historically, PBMCs and CD4+ T cells have been cultured in the presence of FBS. Both FBS-derived TGF-β and TGF-β released by HIV-infected PBMC likely exert an inhibitory effect on HIV reactivation ex vivo in qVOA-type assays. This explains the activity of galunisertib in increasing the frequency of latency reactivation in our qVOA. Importantly, the qVOA was a highly simplified and modified version of the classical qVOA(59). A major limitation of this work is that we have only tested PMA and Vorinostat as LRAs. We did not determine whether inhibiting TGF-β signaling may also increase HIV reactivation following treatment with BRD4 inhibitors, HMT inhibitors or TLR inhibitors. TGF-β1 is known to inhibit TLR-signaling, so it is likely that galunisertib will enhance the LRA activity of TLR agonists. Its impact on other LRA classes is less clear, and will need to be addressed in future studies.

An important result obtained involved the ability of galunisertib to drive SIV reactivation in vivo in aviremic, SIV infected macaques. While a clear SIV env signal was detected in tissues by PET/CT following galunisertib treatment in 5 out of 7 animals, an increase in VL was detected in only 2 out of the 7 animals. A14X, whose viremia had not been continuously suppressed in blood before galunisertib treatment, had a very high (>2 Log) increase in pVL with a relatively low increase in gut SUVtot. However, this macaque had a substantial increase in PET signal in the LNs which may explain the increase in pVL. In contrast, in the other 4 macaques that showed reactivation in tissue there was minimal or no increase in vRNA in blood. Considering that blood was not sampled every day, rather every 3-4 days, it is possible that a transient increase in pVL was not detected. However, it is also possible that viral expression/reactivation was localized in tissues and not enough virus reached the blood to be detected. Importantly, the observed increase in SUVtot in the gut was reflected in the increase in vRNA in colorectal biopsies. This occurred despite the highly focalized sampling of biopsies compared with measuring the overall PET signal in the entire organ, warranting further study and confirmation. However, it is important that the data collected with the two different techniques agree with each other. In contrast, the lack of correlation between increase in PET signal in lymph nodes and the increase in vRNA in FNA may be due to the FNA technique involving sampling a single site, not necessarily axillary, while the PET signal that was isolated with the MIM software encompassed only the axillary LNs. A further confirmation of the value of the increase in PET signal in specific tissue area is given by the analysis of a gut tissue resection that captured an area of PET signal in an SIVmac239 infected macaque (FIG. 13). The SUVtot in each tissue area correlates with the amount of vRNA detected that part of the tissue. The sensitivity of the 64CuDOTA-F(ab′)2 p7D3 probe has not been evaluated in vivo because of its dependency not only on number of SIV-gp120 expressing cells, but also on the amount of gp120 expression and cell density within a specific tissue volume. However, FIG. 13 demonstrates that immunoPET/CT can capture small differences in viral replication at least as well as RT-qPCR would.

Some insights into the dynamics of viral rebound on ART during galunisertib treatment comes from the sequencing done on A14X013. This animal had a large increase in gut SUVtot detected by immunoPET, but no increase in pVL was detected with our sampling schedule. Yet we found evidence of increased viral diversity during galunisertib treatment particularly in blood that concluded with a new dominant viral population closely related to those detected in rectal tissue. Although this analysis was done only in 1 macaque, it suggests that galunisertib may induce viral expansion of multiple infection foci and migration of viral populations between tissue and blood.

Finally, an increase in SIV-specific immune responses was observed that appeared to be driven by the galunisertib treatment. Since these increases were also detected in the animals that exhibited no detectable rebound (A14X005 and A14X037), they may not have been directly related to increases in viral antigen. This is intriguing and suggests potential direct effect of inhibiting TGFBR1 signaling on SIV-specific CD4 and CD8 cells. An increase in T cell function was also detected during the preclinical and clinical development of galunisertib(41, 42). Interestingly, the two animals where reactivation could not be detected had the highest increase in SIV-specific antibody responses. This suggests that the increased SIV-responses may have masked SIV reactivation in these animals. Whether this increased immune response could translate into increased purging of the viral reservoir was not the focus of this first proof-of-concept study and remains to be determined. In any case, the combination of viral reactivation and enhanced cellular and humoral responses in this first pilot study is highly promising and suggests that galunisertib may have an impact on the viral reservoir alone or in combination with other strategies to enhance immune functions killing virus-expressing cells. The studies presented herein have several limitations. They include the use of cell lines as models for HIV latency, which may not fully recapitulate the mechanisms of latency in vivo. Moreover, as mentioned above only PMA and SAHA were tested in combination with galunisertib in a limited number of PBMC samples from aviremic PLWH. Additional studies will be needed to dissect potential differences in the activity of galunisertib on different mechanisms of HIV-1 reactivation. Regarding the in vivo studies, the limited number of animals and lack of prolonged follow-up or ART interruption as well as relatively short treatment course are limitations that can be addressed in future studies now that galunisertib has been determined to be able to mediate virus reactivation. The disclosed dosing was based on pre-clinical studies in other animals and on the therapeutic index used for clinical studies in humans. However, the concentration of galunisertib in the blood of macaques is at least 10-fold lower than the average concentration detected in human plasma after similar dosing. Future work will require more detailed PK/PD studies in macaques. Another drawback of this study is that analysis of cell phenotypes in PBMC and tissues before and after galunisertib administration in vivo are missing. This was not done because of limited sample availability. In future studies, it will be important to determine whether galunisertib treatment leads to an increase in markers of immune activation or an increase in the expression of inflammatory markers. Finally, virus sequencing was limited to 1 macaque and to gag. This limits our ability to draw any conclusion on the effect of galunisertib on viral dynamics. Near full-length viral sequencing should be performed in future studies in all animals to better clarify the impact of galunisertib on viral dynamics during ART. It is noteworthy that viral reactivation did not correlate with higher baseline TGF-β concentrations in blood. Hence, the effect of galunisertib on the virus appears to be independent of TGF-β levels in plasma and the levels in plasma may not directly reflect the levels in tissues. Moreover, we monitored total TGF-β and the levels of active TGF-β may be different. Future studies should include monitoring of TGF-β in tissue at baseline to understand whether galunisertib may work better in the context of high baseline tissue TGF-β levels.

In conclusion, we have demonstrated that TGF-β inhibits HIV reactivation from latently infected cells, an activity that may impact ex vivo assays aimed at quantifying the size and dynamics of reservoirs. We further demonstrate that a small molecule TGFBR1 inhibitor, galunisertib, in advanced stages of clinical development, can promote viral reactivation ex vivo and in vivo. Measurements of pVL did not detect reactivation in vivo in all instances, likely due to insufficient sensitivity. This work demonstrates that immunoPET/CT imaging can provide important additional information regarding the efficacy of HIV-1 cure strategies. Because this was a first attempt at using an antagonist of TGF-β to reactivate the HIV-1 reservoir in vivo, it is may be that improved treatment protocols will lead to improved activity. Nonetheless, these results are both surprising and promising and may lead to novel therapeutic interventions to reduce or eliminate the HIV-1 reservoir and bring the field closer to a cure.

### Materials and Methods

### Study Design

A total of 15 adult male and female Indian Rhesus macaques (Macaca mulatta; Mamu A*01, B*08 and B*17 negative) were used for the in vivo studies described (FIG. 9). Two additional Rhesus macaques A8L057 and 08M171 were infected with SIVmac239M2 (generously provided by Brandon Keele) and used as controls for, respectively, FIG. 4 and FIG. 5 at, respectively, week 56 and 32 post-infection. All rhesus macaques used in these studies were selected form the colonies bred and raised at the New Iberia Research Center (NIRC), University of Louisiana at Lafayette. Treatment of all macaques were in accordance with NIRC standards and any abnormal observations and/or signs of illness or distress were reported per NIRC SOPs. All efforts were made to minimize suffering and stress. Appropriate procedures were performed to ensure that potential distress, pain, discomfort were limited to that unavoidable in the conduct of the research plan. Ketamine (10 mg/kg) and/or telazol (4 mg/kg) were used for sample administration, collection of samples and conducting image collections. Analgesics were used when determined appropriate by veterinary medical staff. Monkeys were fed monkey chow supplemented with fresh fruit or vegetables and water ad libitum.

Rhesus macaques (n=7) were infected with SIVmac251 or SIVmac239 rectally or intravenously (FIG. 9) and ART (Tenofovir [PMPA], Emtricitabine [FTC] and Dolutegravir [DTG]) was initiated between week 12 and 17 post infection. Galunisertib treatment was initiated after 26 or 27 weeks on ART for 7 or 14 days. Galunisertib powder (MedChemExpress—MCE, NJ, USA) was dissolved in water and given orally in a treat twice daily at 5 mg/kg or 10 mg/kg (FIG. 9). Blood viral load was monitored biweekly before and during cART and every 3-4 days during 438 Galunisertib treatment. Colorectal biopsies and LN FNA were collected before and after galunisertib treatment.

PET/CT imaging with a 64Cu-DOTA-F(ab′)2 p7D3 anti SIV-env probe were performed before Galunisertib treatment, after 7 days in all macaques and after 14 days in the macaques that were treated for 14 days. Briefly, a solution of 64Cu was incubated with DOTA-F(ab′)2 p7D3 antibody reconstituted with 50 μL of IM NH40Ac, pH of 5.5 and incubated for 1 hour at 37° C.; then purified by ultrafiltration in a desalting spin column (10K MWCO, Thermo Fisher) and finally eluted with sterile saline prior to injection. The labeled probe was injected IV via catheters, 24 h before PET-CT scanning. For taking PET/CT scans, the macaques were sedated with ketamine HCL (10 mg/kg), and/or telazol (4-8 mg/kg) via intramuscular and supplemented with ketamine (5 mg/Kg) as needed to perform the procedure. The macaque's body was immobilized in dorsal recumbency on the scanner table and PET/CT scans were acquired using a Philips Gemini TF64 PET/CT scanner. The final CT image was compiled from 250 to 300 slices, depending on macaque size.

PET Image analysis was performed using the MIM software. PETCT fusions were generated scaled according to calculated Standardized Uptake Values. The SUV scale for the PET scans was selected based on the overall signal intensity of the PET scans (whole body), and the CT scale was selected for optimal visibility of the tissues. All images and maximum image projections were set to the same scale for visual comparisons.

Regions of interest (ROI) were isolated using a combination of the Region Grow function and manual contouring on a representative scan. These regions were the copied onto subsequent scans of the same animal using a specialized developed workflow. This workflow uses the CT scans to map the selected ROI and locate that corresponding volume in subsequent scans. Manual adjustments were then used to counter any changes in the animals' orientation between scans. The signals of the whole body as well as within these ROI were quantified, and Max, Mean, and Total SUVs were calculated for further analyses. Organs of high and unwanted signal (kidneys and liver) were masked using the Region Grow function for selecting the boundaries of the organ signal and the Mask function to reduce the signal within the contour to background levels (for A14X004 only).

### SIVmac251 and SIVmac239 Preparation and Administration

Viral stocks were propagated and titrated in Rhesus PBMC and prepared for either intrarectal or intravenous dosing in a biosafety cabinet and diluted in medium. Rhesus macaques were inoculated intravenously with 300 TCID50 of SIVmac239 or intrarectally with 2000 TCID50 of SIVmac251 or SIVmac239. Intravenous injections or intrarectal administration were done using 1 mL of diluted virus while macaques were under sedation.

### In Vitro Models of HIV Latency Reactivation

ACH2 cells and U1 cells were obtained through the NIH HIV Reagent Program, Division of AIDS, NIAID, NIH (contributed by Dr. Thomas Folks). Cells (105/well) were cultured in triplicates in RPMI with 10% FBS and Pen/Strep in presence of TGF-β1 (10 ng/ml R&D systems), TGF-β1 and galunisertib (1 μM MedChemExpress), galunisertib alone or mock treated. In the HIV latency reactivation conditions, PMA (Phorbol 12-myristate 13-acetate, SigmaAldrich) was added at 100 ng/ml together with galunisertib or TGF-β1. After 18 hrs, cells were stained intracellularly for p24 using the KC57 antibody (NIH HIV Reagent Program) as previously described (74).

Blood from de-identified donors was obtained from the New York Blood Bank and used as source of CD4 T cells for the primary model of HIV latency in vitro. Total CD4+ T cells were isolated with the CD4 T cells isolation kit (Miltenyi) and activated with 5 μg/ml of PHA and 50 U/ml of IL-2 for 3 days. After washing out the PHA, cells were infected with 200TCID50 of HIV-ADA (NIH HIV Reagent Program) in 12 or 24 wells/plates and spinoculated at 1200 g for 2 hrs at 25° C. After additional 3 hrs of incubation at 37° C., viral inoculum was washed and cells plated in presence of T20 (Enfurtivide acetate salt, 1 μM NIH AIDS reagent program). After 48 hrs, cells were treated with PMA (100 ng/ml) in presence and absence of galunisertib (1 μM) overnight and stained for p24 intracellularly as described above.

CD14⁺ cells were isolated from PBMC using the Miltenyi CD14⁺ beads. Monocyte-derived DCs (moDCs) were obtained from CD14+ cells cultured in GM-CSF and IL-4 as previously described (55). At day 4 of moDCs differentiation, TGF-β1 (10 ng/ml R&D systems) was added to the moDCs to generate the TGF-β-DCs (or both TGF-β1 and galunisertib). At day 6, 2 days after TGF-1 addition, moDCs were phenotyped (in 3 donors; See Table 2 for antibodies list) and co-cultured with eFluor670 (eBioscience)-labelled U1 at 3:1 U1:DC ratio (5 donors). After 18 hrs of co-culture, cells were stained for p24 intracellularly as described above.

TABLE 2
List of antibodies used for moDC phenotyping and PBMC ICS.

| Marker | Dye | Clone | Manufacturer |
|---|---|---|---|
| HLA-DR | BV605 | L243 | BioLegend |
| CD54 | AF700 | HA58 | BioLegend |
| CD106 | PE | STA | BioLegend |
| CD209 | BV421 | 9E9A8 | BioLegend |
| CD169 | PEcy7 | 7239 | BioLegend |
| DEC205 | AF647 | MMRI7 | BioLegend |
| CD3 | V450 | SP34-2 | BD Biosciences |
| CD4 | BUV395 | L200 | BD Biosciences |
| CD8 | PCP-Cy5.5 | SK1 | BD Biosciences |
| IFN-γ | Alexa Fluor 700 | B27 | BD Biosciences |
| TNF-α | FITC | Mab11 | BioLegend |
| IL-2 | BV605 | MQ1-17H12 | BioLegend |

### Quantitative Viral Outgrowth Assay

The standard qVOA assay (59) was extensively modified. PBMC from aviremic donors were used in absence of resting CD4+ T cells isolation. De-identified PBMC were obtained either from NIAID protocol 14-I-0039 or 10-I-N048 or from the Northwestern University RADAR cohort.

PBMC were thawed and cultured in RPMI 10% FBS at 3×10⁶/ml. Cells were activated with PMA (100 ng/ml or 10 ng/ml) or Vorinostat (1 μM, NIH HIV Reagent Program) in presence of T20 (HIV Reagent Program) and IL-2 (100 U/ml) and galunisertib (1 μM) or mock (DMSO). After 18 hrs, cells were washed twice in PBS and plated at 3×10⁵/well (>14 replicate wells; a number where we expected <1 HIV latently infected cell/well (75)) in a flat-bottom 96 wells/plate. 105 SupT1-R5 (M10 clone, HIV Reagent Program) were added in each well and galunisertib (1 μM) or an equal volume of DMSO were added to the respective wells in each condition. 50 μl of supernatant was collected after 7, 14 and 21 days of co-culture and monitored for p24 presence by p24 ELISA (PerkinElmer). IUPM were calculated based on the number of positive wells using the IUPM calculator provided by Dr. Siliciano (76). Supernatant from stimulated PBMC before co-culture with SupT1 cells was tested by gag-RT-qPCR (77).

For control experiments of the impact of galunisertib on HIV infection of SupT1 cells and PBMC, SupT1 cells and PHA-activated PBMC were cultured respectively at 1 and 2 millions/ml in R10 (in presence of 20 U/ml of IL-2 for the PBMC) and infected with 50 TCID50/10⁶ cells of HIV-Bal (0.01 MOI). HIV replication was monitored by p24 ELISA.

### Plasma and Tissue SIV Viral Loads

Blood was collected in EDTA tubes and plasma was separated by density gradient centrifugation and used for the determination of plasma VL by SIVgag qRT-PCR at NIRC or at Leidos (Quantitative Molecular Diagnostics Core, AIDS and Cancer Virus Program Frederick National Laboratory). Tissue VL from snap frozen colorectal biopsies and LN FNA were performed at Leidos (A14X004, A14X005 and A14X013) as described in (78) or, for the remaining macaques and from PBMC of all macaques they were performed as described in (77).

### Galunisertib Pharmokinetics and Pharmacodynamics in Macaque Blood

Eight naïve macaques (FIG. 9) were used to collect plasma and PBMC for PK/PD studies. 4 macaques received galunisertib (2.5 mg/kg, n=2 or 5 mg/kg, n=2) once and blood was collected at 1 hr, 3 hr and 6 hrs, 24 hrs and 48 hrs. 4 macaques received galunisertib (2.5 mg/kg, n=2 or 5 mg/kg, n=2) twice daily for 9 days and samples were collected 1 hr, 3 hr and 8 hrs after administration on day 1 and on day 9 right before and 1 hr and 3 and 6 hrs after the last administration. Plasma concentrations of galunisertib were measured by IMSERC (Integrated Molecular Structure Education and Research Center) utilizing liquid chromatography mass spectrometry (Shimadzu Kyoto, Japan ultra-high performance liquid chromatography—UHPLC—system and SCIEX Framingham, MA Qtrap 6500+ triple quadrupole mass spectrometer). All standard, quality controls (QC) and experimental samples were prepared and ran in duplicate using 20 μL of plasma (spiked with galunisertib if a standard or QC) adding 15 μL of 8% phosphoric acid solution, 200 μL of acetonitrile (to crash plasma proteins) spiked with tramadol as an internal standard. The standard curve was prepared as a 10-point curve from 1.0 to 1,000 ng/ml with three QCs. For the LCMS method, a 1 μL injection was performed of the resulting supernatant separated on a Phenomenex (Torrance, CA) Kinetex® Biphenyl 2.6 μm 100A LC column (50×2.1 mm). The separation gradient was performed using 0.1% formic acid in water (A) and 0.1% formic acid in methanol (B) mobile phases following a gradient at 0.6 mL/min flow rate of: 0.5 minutes 10% B, 4.0 minutes 85% B and hold for 1.0 minutes, and returning to 10% B at 5.1 minutes holding for 1.4 minutes. All samples and standards were processed in duplicate with the average area under the curve (AUC) being used for quantitation reporting. After internal standard correction, all samples and standards outside of a 20% CV between preparation replicates were rerun in duplicate to meet this requirement. The resulting limit of quantitation (LOQ) was 1.0 ng/ml.

For PD studies, the levels of phosphorylated SMAD2/3 were measured using the PathScan PhospoSMAD2/3 ELISA kit (Cell Signaling Technology, CST) over the levels of Total SMAD2/3 in cell lysates Total SMAD ELISA Kit (Cell Signaling Technology, CST) normalized on protein content.

Sequencing of SIV Genome in PBMV, Rectal Biopsies, and Plasma from A14X013

DNA was extracted from PBMC and rectal biopsies using the DNAeasy blood and tissue kit (Qiagen), while RNA was extracted from plasma using the QIAamp Viral RNA Mini Kit (Qiagen). Extracted RNA was immediately reverse transcribed using SuperScript™ VILO™ CDNA Synthesis Kit (Invitrogen). Q5® Hot Start High-Fidelity 2X Master Mix (NEB) was used to amplify the gag gene with primers Outer-Gag-F (5′-GAAGCAGGAAAATCCCTAGCAG-3′) and 8R (5′—CCAACTGACCATCCTTTTCCATCTTT-3′) followed by a second amplification round using primers 1F (5′-TCCTGAGTACGGCTGAGTGAAG-3′) and 7R (5′-TCCTATTCCTCCTACTATTTTTGGGGT-3′) (79). PCR cycling conditions were as follows: 98° C. for 30 s, 35 cycles of 15 s at 95° C., 30 s at 65° C., 2 min at 72° C. and a final extension of 72° C. for 2 min. Sequencing library preparation of the obtained amplicons was performed using the SeqWell plex Well 384 kit per manufacturer's instructions. Pooled libraries were sequenced on the Illumina MiSeq using the V2 500 cycle kit. We subsequently performed reference-based mapping assembly of the sequencing reads using the HAPHPIPE protocol and SIVmac239 reference genome (M33262.1). With this protocol, reads were trimmed to remove adapters and low-quality sequences using Trimmomatic v0.39 and error-corrected using SPAdes v3.13.1. PBMC samples were sequenced twice to confirm the results obtained. Sequences were deposited in GenBank: OP390088-OP390161.

### Phylogenetic and Diversity Analysis

With the assembled reads we performed probabilistic inference of intra-host viral quasispecies for each sample obtained from A14X013 using QuasiRecomb. The sequences of the inferred viral haplotypes from each quasispecies were aligned using MAFFT v7.453 software and a Maximum Likelihood (ML) phylogeny was reconstructed with IQ-Tree v2.0.5 to track the spatio-temporal evolution of the intra-host viral populations. The final tree representation was performed with the R package ggtree v3.2.1 (doi.org/10.1111/2041-210X.12628). Intra-host diversity for each sample analyzed was calculated using DistanceCalculator in Biopython and pairwise distances weighted by the estimated frequency of the haplotype with an in-house python script.

### Anti-SIV T Cell Responses

Frozen PBMC collected right before the first galunisertib administration and right after were thawed in AIM V medium (Thermo Fisher) plated on a plate pre-coated with 2.5 μg/ml goat anti mouse (GAM) IgGs and cross linked with 10 μg/ml anti-CD28 and anti-CD49d antibodies (Sigma Aldrich). Cells were stimulated either with SIVMAC239 GAG peptide pool (2 μg/ml) or with with SIVMAC239 ENV peptide pool (2 μg/ml AIDS reagents program, Division of AIDS, NIAID, NIH) or the same volume of DMSO as baseline control. Pooled cells stimulated with 1 μg/ml PMA/Ionomycin were used a positive activation control. One hour later 10 μg/ml BFA and monensin (GolgiStop, BD Biosciences) were added to each well. After 5 hours, cells were transferred to a FACS plate and stained with the LIVE/DEAD Aqua viability dye (Thermo Fisher Scientific) and antibodies against cell surface and cytokines (Table 2).

### Anti-Envelope Antibodies

96-well plates were coated with the SIV gp130 Recombinant protein 595 (AIDS reagents program, Division of AIDS, NIAID, NIH at 1 μg/ml in coating carbonate buffer 2 hours at room temperature (RT). After washing the plates 4 times with washing buffer, the plates were incubated in blocking buffer (2% BSA in PBS) overnight. Plates were washed and dilutions of test sera or control sera were added to the plate starting at 1:50. Sera were incubated at 37° C. for 30′ and plate washed and detected with anti-Rhesus IgG HRP (NHPRR) 30′ at 37° C. After washing and substrate incubation at RT for 30′ plates were read at 450/650 nm.

### Statistics

Data from different conditions of ACH-2 and U1 experiments were compared using the Kruskal-Wallis ANOVA test followed by the Dunn's test corrected for multiple comparisons. For the QVOA experiments, IUPM with and without galunisertib were compared in the LRA different conditions using 2-way ANOVA followed by the Sidak multiple comparisons test. Before and after galunisertib SUV, tissue VL data as well as anti-envelope titers and frequency of cells producing different cytokines combination following peptide pool stimulation were compared using a mixed-effect model with the Sidak multiple comparisons test, with a singled pooled variant. DC phenotype between TGF-β-DC and moDCs was compared using the ratio paired t-test. Statistical analysis was performed using Prism GraphPad 9.3 and R.

### REFERENCES

1. Siliciano J D, Siliciano R F. Recent developments in the search for a cure for HIV-1 infection: Targeting the latent reservoir for HIV-1. J Allergy Clin Immunol. 2014; 134(1): 12-9.
2. Yukl S A, et al. HIV latency in isolated patient CD4(+) T cells may be due to blocks in HIV transcriptional elongation, completion, and splicing. Sci Transl Med. 2018; 10(430).
3. Halvas E K, et al. HIV-1 viremia not suppressible by antiretroviral therapy can originate from large T cell clones producing infectious virus. J Clin Invest. 2020; 130(11):5847-57.
4. Kruize Z, Kootstra N A. The Role of Macrophages in HIV-1 Persistence and Pathogenesis. Frontiers in microbiology. 2019; 10:2828.
5. Abreu C M, et al. Myeloid and CD4 T Cells Comprise the Latent Reservoir in Antiretroviral Therapy-Suppressed SIVmac251-Infected Macaques. mBio. 2019; 10(4).
6. Ganor Y, et al. HIV-1 reservoirs in urethral macrophages of patients under suppressive antiretroviral therapy. Nat Microbiol. 2019; 4(4):633-44.
7. Murray J M. Latent HIV dynamics and implications for sustained viral suppression in the absence of antiretroviral therapy. J Virus Erad. 2018; 4(2):91-8.
8. Assoumou L, et al. A low HIV-DNA level in peripheral blood mononuclear cells at antiretroviral treatment interruption predicts a higher probability of maintaining viral control. AIDS. 2015; 29(15):2003-7.
9. Fidler S, et al. Virological Blips and Predictors of Post Treatment Viral Control After Stopping ART Started in Primary HIV Infection. Journal of acquired immune deficiency syndromes (1999). 2017; 74(2): 126-33.
10. Singh V, et al. Latency Reversal 2.0: Giving the Immune System a Seat at the Table. Curr HIV AIDS Rep. 2021; 18(2): 117-27.
11. Abner E, Jordan A. HIV “shock and kill” therapy: In need of revision. Antiviral Res. 2019; 166: 19-34.
12. Ait-Ammar A, et al. Current Status of Latency Reversing Agents Facing the Heterogeneity of HIV-1 Cellular and Tissue Reservoirs. Frontiers in microbiology. 2019; 10:3060.
13. Siliciano J D, Siliciano R F. Low Inducibility of Latent Human Immunodeficiency Virus Type 1 Proviruses as a Major Barrier to Cure. J Infect Dis. 2021; 223(12 Suppl 2): 13-21.
14. Ho Y C, Shan L, et al. Replication-competent noninduced proviruses in the latent reservoir increase barrier to HIV-1 cure. Cell. 2013; 155(3):540-51.
15. Biancheri P, et al. The role of transforming growth factor (TGF)-beta in modulating the immune response and fibrogenesis in the gut. Cytokine Growth Factor Rev. 2014; 25(1):45-55.
16. Konkel J E, Chen W. Balancing acts: the role of TGF-beta in the mucosal immune system. Trends Mol Med. 2011; 17(11):668-76.
17. Huang F, Chen Y G. Regulation of TGF-beta receptor activity. Cell Biosci. 2012; 2:9.
18. Xu J, Lamouille S, and Derynck R. TGF-beta-induced epithelial to mesenchymal transition. Cell Res. 2009; 19(2):156-72.
19. Gorelik L, Flavell R A. Abrogation of TGFbeta signaling in T cells leads to spontaneous T cell differentiation and autoimmune disease. Immunity. 2000; 12(2): 171-81.
20. Li M O, et al. Transforming growth factor-beta regulation of immune responses. Anmu Rev Immunol. 2006; 24:99-146.
21. Classen S, et al. Human resting CD4+ T cells are constitutively inhibited by TGF beta under steady-state conditions. J Immunol. 2007; 178(11):6931-40.
22. Zhang N, Bevan M J. TGF-beta signaling to T cells inhibits autoimmunity during lymphopenia driven proliferation. Nat Immunol. 2012; 13(7):667-73.
23. Nandan D, Reiner N E. TGF-beta attenuates the class II transactivator and reveals an accessory pathway of IFN-gamma action. J Immunol. 1997; 158(3): 1095-101.
24. Sanjabi S, et al. Regulation of the Immune Response by TGF-beta: From Conception to Autoimmunity and Infection. Cold Spring Harb Perspect Biol. 2017; 9(6).
25. Wiercinska-Drapalo A, et al. Increased plasma transforming growth factor-beta1 is associated with disease progression in HIV-1-infected patients. Viral Immunol. 2004; 17(1): 109-13.
26. Johnson M D, et al. Transforming growth factor beta and monocyte chemotactic protein-1 are elevated in cerebrospinal fluid of immunocompromised patients with HIV-1 infection. J NeuroAIDS. 2004; 2(4):33-43.
27. Maina E K, et al. Plasma concentrations of transforming growth factor beta 1 in non progressive HIV-1 infection correlates with markers of disease progression. Cytokine. 2016; 81:109-16.
28. Estes J D, et al. Antifibrotic therapy in simian immunodeficiency virus infection preserves CD4+ T-cell populations and improves immune reconstitution with antiretroviral therapy. The Journal of infectious diseases. 2015; 211(5): 744-54.
29. Reinhold D, et al. HIV-1 Tat: immunosuppression via TGF-beta1 induction. Immunol Today. 1999; 20(8):384-5.
30. Hu R, et al. HIV-1 gp160 induces transforming growth factor-beta production in human PBMC. Clin Immunol Immunopathol. 1996; 80(3 Pt 1):283-9.
31. Theron A J, et al. The Role of Transforming Growth Factor Beta-1 in the Progression of HIV/AIDS and Development of Non-AIDS-Defining Fibrotic Disorders. Frontiers in immunology. 2017; 8:1461.
32. Schacker T W, et al. Collagen deposition in HIV-1 infected lymphatic tissues and T cell homeostasis. The Journal of clinical investigation. 2002; 110(8): 1133-9.
33. Schacker T W, et al. Amount of lymphatic tissue fibrosis in HIV infection predicts magnitude of HAART-associated change in peripheral CD4 cell count. Aids. 2005; 19(18):2169-71.
34. Yoshimura A, Muto G. TGF-beta function in immune suppression. Curr Top Microbiol Immunol. 2011; 350:127-47.
35. Tu E, et al. T Cell Receptor-Regulated TGF-beta Type I Receptor Expression Determines T Cell Quiescence and Activation. Immunity. 2018; 48(4):745-59 e6.
36. Dobrowolski C, et al. Entry of Polarized Effector Cells into Quiescence Forces HIV Latency. MBio. 2019; 10(2).
37. Kulpa D A, et al. Differentiation into an Effector Memory Phenotype Potentiates HIV-1 Latency Reversal in CD4(+) T Cells. J Virol. 2019; 93(24).
38. Chinnapaiyan S, et al. TGF-beta1 increases viral burden and promotes HIV-1 latency in primary differentiated human bronchial epithelial cells. Scientific reports. 2019; 9(1): 12552.
39. Kulkarni A B, et al. Transforming growth factor beta 1 null mutation in mice causes excessive inflammatory response and early death. Proc Natl Acad Sci USA. 1993; 90(2): 770-4.
40. Huang T, et al. Biological activity differences between TGF-beta1 and TGF-beta3 correlate with differences in the rigidity and arrangement of their component monomers. Biochemistry. 2014; 53(36):5737-49.
41. Holmgaard R B, et al. Targeting the TGFbeta pathway with galunisertib, a TGFbetaRI small molecule inhibitor, promotes anti-tumor immunity leading to durable, complete responses, as monotherapy and in combination with checkpoint blockade. J Immunother Cancer. 2018; 6(1):47.
42. Herbertz S, et al. Clinical development of galunisertib (LY2157299 monohydrate), a small molecule inhibitor of transforming growth factor-beta signaling pathway. Drug Des Devel Ther. 2015; 9:4479-99.
43. Harding J J, et al. Phase 1b study of galunisertib and ramucirumab in patients with advanced hepatocellular carcinoma. Cancer Med. 2021; 10(9):3059-67.
44. Melisi D, et al. Safety and activity of the TGFbeta receptor I kinase inhibitor galunisertib plus the anti-P D-L1 antibody durvalumab in metastatic pancreatic cancer. J Immunother Cancer. 2021; 9(3).
45. Adams B. Eli Lilly cuts 3 cancer drugs amid Q4 clear-out. fiercebiotech.com/biotech/eli-lilly-cuts-three-cancer-drugs-amid-q4-clear out.
46. Folks™, et al. Cytokine-induced expression of HIV-1 in a chronically infected promonocyte cell line. Science. 1987; 238(4828):800-2.
47. Clouse K A, et al. Monokine regulation of human immunodeficiency virus-1 expression in a chronically infected human T cell clone. J Immunol. 1989; 142(2):431-8.
48. Symons J, et al. HIV integration sites in latently infected cell lines: evidence of ongoing replication. Retrovirology. 2017; 14(1):2.
49. Emiliani S, et al. A point mutation in the HIV-1 Tat responsive element is associated with postintegration latency. Proc Natl Acad Sci USA. 1996; 93(13):6377-81.
50. van Montfort T, et al. Dendritic cells potently purge latent HIV-1 beyond TCR-stimulation, activating the PI3K-Akt-mTOR pathway. EBioMedicine. 2019; 42:97-108.
51. Kristoff J, et al. Role of Dendritic Cells in Exposing Latent HIV-1 for the Kill. Viruses. 2019; 12(1).
52. Ohtani T, et al. TGF-beta1 dampens the susceptibility of dendritic cells to environmental stimulation, leading to the requirement for danger signals for activation. Immunology. 2009; 126(4):485-99.
53. Speck S, et al. TGF-beta signaling initiated in dendritic cells instructs suppressive effects on Th17 differentiation at the site of neuroinflammation. PLOS One. 2014; 9(7):e102390.
54. van der Sluis R M, et al. Dendritic cell-induced activation of latent HIV-1 provirus in actively proliferating primary T lymphocytes. PLOS pathogens. 2013; 9(3):e1003259.
55. Guerra-Perez N, et al. Retinoic acid imprints a mucosal-like phenotype on dendritic cells with an increased ability to fuel HIV-1 infection. Journal of immunology. 2015; 194(5):2415-23.
56. Gupta D, et al. HIV-1 infected peripheral blood mononuclear cells modulate the fibrogenic activity of hepatic stellate cells through secreted TGF-beta and JNK signaling. PLOS One. 2014; 9(3):e91569.
57. Navikas V, et al. Increased levels of interferon-gamma (IFN-gamma), IL-4 and transforming growth factor-beta (TGF-beta) mRNA expressing blood mononuclear cells in human HIV infection. Clin Exp Immunol. 1994; 96(1): 59-63.
58. Kekow J, et al. Transforming growth factor beta and noncytopathic mechanisms of immunodeficiency in human immunodeficiency virus infection. Proceedings of the National Academy of Sciences of the United States of America. 1990; 87(21): 8321-5.
59. Laird G M, et al. Rapid quantification of the latent reservoir for HIV-1 using a viral outgrowth assay. PLOS pathogens. 2013; 9(5):e1003398.
60. Melisi D, et al. Galunisertib plus gemcitabine vs. gemcitabine for first-line treatment of patients with unresectable pancreatic cancer. Br J Cancer. 2018; 119(10): 1208-14.
61. Gueorguieva I, et al. Defining a therapeutic window for the novel TGF-beta inhibitor LY2157299 monohydrate based on a pharmacokinetic/pharmacodynamic model. Br J Clin Pharmacol. 2014; 77(5):796-807.
62. Santangelo P J, et al. Whole-body immunoPET reveals active SIV dynamics in viremic and antiretroviral therapy-treated macaques. Nat Methods. 2015; 12(5):427-32.
63. Santangelo P J, et al. Early treatment of SIV+ macaques with an alpha4beta7 mAb alters virus distribution and preserves CD4(+) T cells in later stages of infection. Mucosal immunology. 2017.
64. Telwatte S, et al. Gut and blood differ in constitutive blocks to HIV transcription, suggesting tissue-specific differences in the mechanisms that govern HIV latency. PLOS pathogens. 2018; 14(11):e1007357.
65. Telwatte S, et al. Mechanistic differences underlying HIV latency in the gut and blood contribute to differential responses to latency-reversing agents. AIDS. 2020; 34(14):2013-24.
66. Chomont N, et al. HIV reservoir size and persistence are driven by T cell survival and homeostatic proliferation. Nature medicine. 2009; 15(8):893-900.
67. Bosque A, et al. Homeostatic proliferation fails to efficiently reactivate HIV-1 latently infected central memory CD4+ T cells. PLOS Pathog. 2011; 7(10):e1002288.
68. Liu R, et al. The forces driving clonal expansion of the HIV-1 latent reservoir. Virol J. 2020; 17(1):4.
69. Maldarelli F, et al. HIV latency. Specific HIV integration sites are linked to clonal expansion and persistence of infected cells. Science. 2014; 345(6193): 179-83.
70. Bushman F D. Retroviral Insertional Mutagenesis in Humans: Evidence for Four Genetic Mechanisms Promoting Expansion of Cell Clones. Mol Ther. 2020; 28(2):352-6.
71. Delisle J S, et al. The TGF-beta-Smad3 pathway inhibits CD28-dependent cell growth and proliferation of CD4 T cells. Genes Immun. 2013; 14(2): 115-26.
72. Nguyen T P, Sieg S F. TGF-beta inhibits IL-7-induced proliferation in memory but not naive human CD4(+) T cells. Journal of leukocyte biology. 2017; 102(2):499-506.
73. Xu X, et al. Transforming growth factor-beta in stem cells and tissue homeostasis. Bone Res. 2018; 6:2.
74. Goode D, et al. HSV-2-driven increase in the expression of alpha4beta7 correlates with increased susceptibility to vaginal SHIV(SF162P3) infection. PLOS pathogens. 2014; 10(12):e1004567.
75. Finzi D, et al. Identification of a reservoir for HIV-1 in patients on highly active antiretroviral therapy. Science. 1997; 278(5341): 1295-300.
76. Rosenbloom D I, et al. Designing and Interpreting Limiting Dilution Assays: General Principles and Applications to the Latent Reservoir for Human Immunodeficiency Virus-1. Open Forum Infect Dis. 2015; 2(4): ofv123.
77. Frank I, et al. Blocking alpha4beta7 integrin delays viral rebound in SHIVSF162P3-infected macaques treated with anti-HIV broadly neutralizing antibodies. Sci Transl Med. 2021; 13(607).
78. Swanstrom A E, et al. Antibody-mediated depletion of viral reservoirs is limited in SIV956 infected macaques treated early with antiretroviral therapy. The Journal of clinical investigation. 2021; 131(6).
79. Moriarty R V, et al. Validation of multiplex PCR sequencing assay of SIV. Virol J. 2021; 18(1):21.

### TGF-β Blockade Drives a Transitional Effector Phenotype in Immune Cells

HIV-1 persistence early after infection (80, 81), and during ART, is due to the establishment of long-lived viral reservoirs in resting immune cells. A key mechanism of this persistence is the ability of HIV-1 to enter a state of virological latency characterized by the silencing of viral gene expression and/or lack of viral proteins translation (82, 83). This allows the virus to remain invisible to the immune system and latently infected cells to survive and proliferate by homeostatic or antigen-driven proliferation(84, 69). Of note, the viral reservoir was initially thought to be stable. However, recent evidence suggests that stochastic HIV reactivation under ART occurs, and selective killing is favored in cells bearing replication competent virus integrated in transcriptionally active sites within the genome(85-87). Hence, integration site, but especially the activation status of the infected cells profoundly influences HIV-1 persistence.

Ongoing efforts to achieve a functional cure for HIV-1 are directed towards supplementing ART with immunotherapies targeting the viral reservoir. Such strategies, under the umbrella of “shock and kill,” aim to reactivate the replication competent reservoir and elimate latently infected cells by viral cytopathic effects or immune-mediated killing (10, 88). However, these strategies have thus far failed to achieve a reduction of the viral reservoir or post-ART virologic control in either the clinical or preclinical models. This is mainly due to the low inducibility of latent proviruses and the heterogeneity of the mechanisms of persistence (12, 2, 13, 14). In contrast, therapeutic vaccination strategies focused on enhancing HIV/SIV-specific responses have had some discreet measure of success (89). However, currently no single strategy leads simultaneously to latency reversal and stimulation of effective immune responses.

The HIV life cycle and HIV's ability to replicate efficiently are especially dependent on the activation status of the infected cells. In this context, significant advances have been made to directly activate immune cells through non-canonical pathways in order to promote HIV latency reversal(90). However, the activation and differentiation of immune cells is intrinsically linked to cellular metabolism(91). Indeed, signaling pathways that govern immune cell differentiation and activation such as mTOR and Wnt/β-catenin, not only have been implicated in regulating HIV latency(92-94), but are also critical regulators of cell metabolism(94). The metabolic status of an infected cell, in turn, plays a critical role in its ability to support latency reactivation and viral production(92, 96).

In this context, recent evidence suggests that TGF-β plays a critical role in the regulation of immune cell activation and metabolic reprogramming(97-99). Specifically, in the context of CD8+ T cells, TGF-β has been shown to suppress mTOR signaling preserving the metabolic fitness of memory CD8+ T cells(98) and stem-like antigen specific CD8+ T cells through its modulation of Wnt/β-catenin factor TCF1(99, 100). TCF1 and the mTOR pathway are also critical to T cell differentiation and responsible for the transition from activated effector cells to resting memory cells during LCMV infection(95, 99). The regulation of quiescence in CD8+ T cells that follows continuous TGF-β stimulation is critical to the transition to a memory phenotype and it is driven by specific metabolic changes that are linked to decreased glycolytic activity, more efficient mitochondrial respiration, and long-term survival(98, 99)

Similarly, in the context of NK cells, TGF-β has been implicated in decreasing their baseline metabolism driving lower expression of markers of NK cytotoxic activity(101). While TGF-β-mediated suppression of TCR and IL-2 signaling were shown to lead to lower CD4+ T cell activation following cognate antigen recognition in older studies(97, 71), more recent work using CD4+ T cell-specific deletion of the TGF-β receptor demonstrated an even more profound effect of TGF-β on all stages of CD4+ T cell activation, proliferation and cytotoxic response to LCMV than in CD8+ T cells(102). However, little is known on the specific role of TGF-β in regulating the transition to and from memory and effector phenotypes in CD4+ T cells and how this may be associated with TGF-β-driven changes in CD4+ T cells metabolism. Moreover, TGF-β regulates the expression of CD103 and other surface and intracellular factors essential of T cell residency in mucosal tissues(103-105). Hence, TGF-β is considered the master regulator of mucosal immunity(106).

Above it was demonstrated that TGF-β regulates HIV-1 latency in primary CD4+ T cells ex vivo and in vivo. Herein, it is demonstrate how treatment with galunisertib with a 2-week on, 2-week off regiment that mimics the therapeutic regimen employed in the clinic in phase 1 and 2 trials of solid tumors(44, 109), leads to profound transcriptional and functional changes in immune cells in the absence of overt toxicity or increased systemic inflammation. Importantly, we observed a shift toward a transitional effector phenotype in CD4+ T cells and other immune cells both systemically and in the lymph nodes. This shift was accompanied by, and likely responsible for, increased viral reactivation in SIV-infected, ART treated macaques documented by molecular techniques and PET/CT images. At the end of the treatment with galunisertib, lower viral reservoir levels, including total and intact proviral DNA in both PBMC, gut and lymph nodes and significantly higher immune responses were detected.

Using an NHP model of barcoded SIVmac239 intravenous infection and therapeutic dosing of galunisertib (LY2157299), the latency reversal properties of in vivo TGF-β blockade, decreased viral reservoirs and stimulated immune responses were confirmed. Eight SIV-infected macaques on suppressive ART were treated with four 2-week cycles of galunisertib. ART was discontinued 3 weeks after the last dose, and macaques euthanized 6 weeks after ART-interruption(ATI). One macaque did not rebound, while the remaining rebounded between week 2 and 6 post-ATI. Galunisertib led to viral reactivation as indicated by plasma viral load and immunoPET/CT with the 64Cu-DOTA-F(ab′)2-p7D3-probe. Half to 1 Log decrease in cell-associated (CA—)SIV DNA was detected in lymph nodes, gut and PBMC, while intact pro-virus in PBMC decreased by 3-fold. No systemic increase in inflammatory cytokines was observed High-dimensions cytometry, bulk and single-cell RNAseq revealed a shift toward an effector phenotype in T and NK cells. In summary, these studies demonstrated that galunisertib, a clinical stage TGF-β inhibitor, reverses SIV latency and decreases SIV reservoirs by driving T cells toward an effector phenotype, enhancing immune responses in vivo in absence of toxicity.

### Example 6—Two Weeks on-Off Therapeutic Regiment with Galunisertib Leads to Viral Reactivation SIV Infected, ART-Treated Macaques

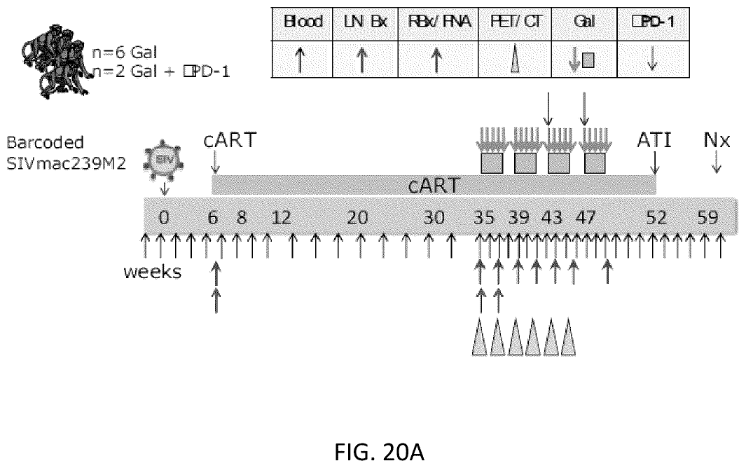

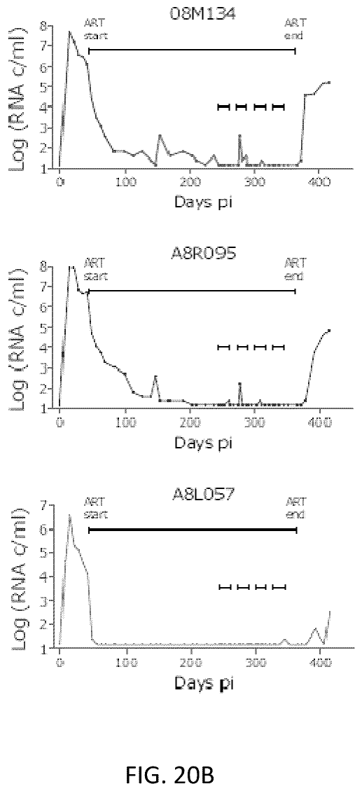

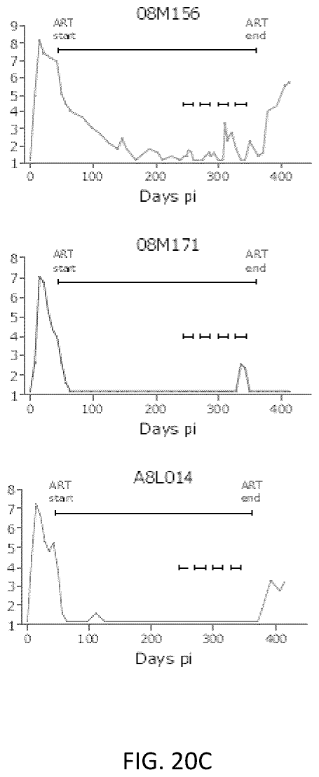

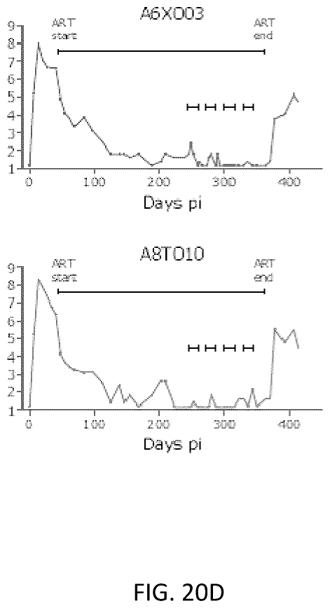

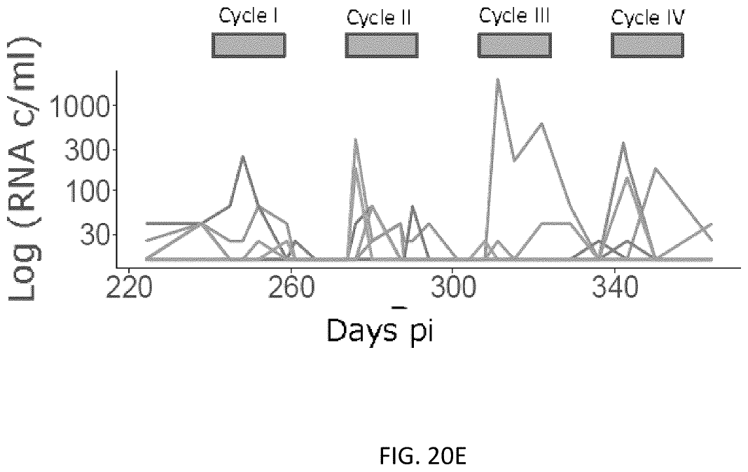

To confirm galunisertib-driven HIV/SIV latency reversal and investigate the underlying mechanisms, 8 Indian origin rhesus macaques (Macaca Mulatta, Mamu-A01-, -B08, -B17-, all females) were infected intravenously with 300 TCID50 of the barcoded SIVmac239M2. We initiated ART treatment (daily co-formulated Tenofovir [PMPA], Emtricitabine [FTC] and Dolutegravir [DTG]) on week 6 post-infection (pi). A 2-week on, 2-week off therapeutic cycle with galunisertib (20 mg/Kg twice/daily orally) started at week ˜35 pi and continued for a total of 4 cycles (FIG. 20A and Table 3). ART was discontinued 3 weeks after the last galunisertib dose, and the macaques were followed for 6 weeks after ART discontinuation. Median peak plasma viral load (pVL) was 108 copies/ml at week 2 pi. A rhesus anti-PD1 antibody was administered at 5 mg/kg before the 3rd and 4th cycle to 2 macaques (08M156 and A6X003). However, no differences were noted for these 2 macaques in any of the parameters we measured, and the data were pooled.

TABLE 3
List of macaques used for the study

| | | | | | | | Cycle 1 | Cycle 4 |
|---|---|---|---|---|---|---|---|---|
| | | | | | | ART- | Start | End |
| | | | | | | Start | (BC1) | (AC4) |
| | Age | | Weight* | Infection | | Week | Week | Week |
| Table S1 | (yo)* | Sex | (Kg) | & Route | Treatment | pi | pi | pi |
| 08M134 | 13 | F | 13.55 | SIVmac239M2 IV | Gal | 6 | 35 | 49 |
| 08M156 | 13 | F | 6.10 | SIVmac239M2 IV | Gal + Nivo | 6 | 35 | 49 |
| A6X003 | 15 | F | 7.85 | SIVmac239M2 IV | Gal + Nivo | 6 | 35 | 49 |
| A8L014 | 13 | F | 7.70 | SIVmac239M2 IV | Gal | 6 | 35 | 49 |
| A8L057 | 13 | F | 9.50 | SIVmac239M2 IV | Gal | 6 | 35 | 49 |
| A8R095 | 13 | F | 8.20 | SIVmac239M2 IV | Gal | 6 | 35 | 49 |
| ABT010 | 13 | F | 5.05 | SIVmac239M2 IV | Gal | 6 | 35 | 49 |
| 08M171 | 13 | F | 7.35 | SIVmac239M2 IV | Gal | 6 | 32 | 46 |
| Average | 13.2 | | 8.12 | | | | | |

*At beginning of the study

Full suppression to undetectable levels (pVL LOD 15 copies/ml) was achieved in 3 out of the 8 macaques at week 10 pi. In the other 5 macaques, pVL fell below 65 copies/ml by week 22 pi with a single blip of 400 copies/ml in A8T010 at week 29 pi (FIGS. 20B-20D). Following the start of the galunisertib treatment, pVLs increased in 7 out of 8 macaques from a single peak over undetectable in 08M171 and A8L057 to several peaks and up to 103 copies/ml in the other macaques. Of note pVLs in A8T010 and A8R095 was undetectable for over 5 weeks before, respectively, blips of up to 102 copies/ml were detected following galunisertib treatment initiation (FIGS. 20B-20D). More frequent blips were noted during the first 2 cycles with galunisertib compared to cycles 3 and 4 (FIG. 20E). However, 08M171 and A8L057 did not experience a pVL increase until the 4th cycle.

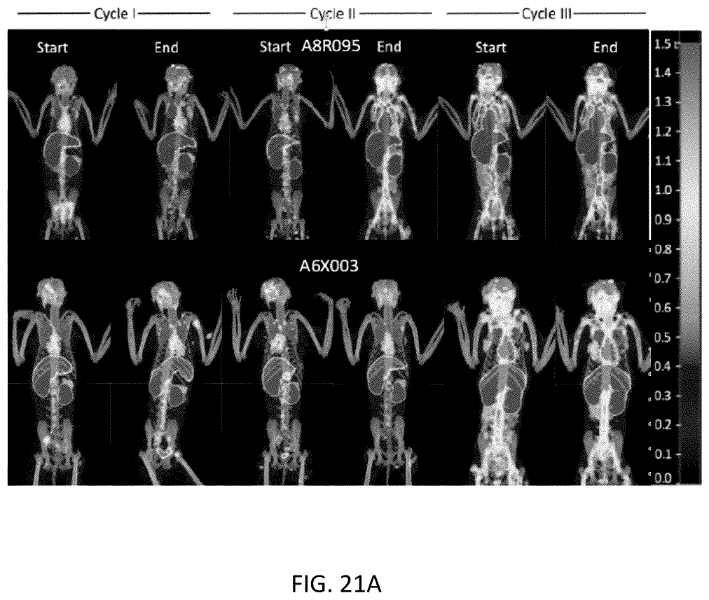

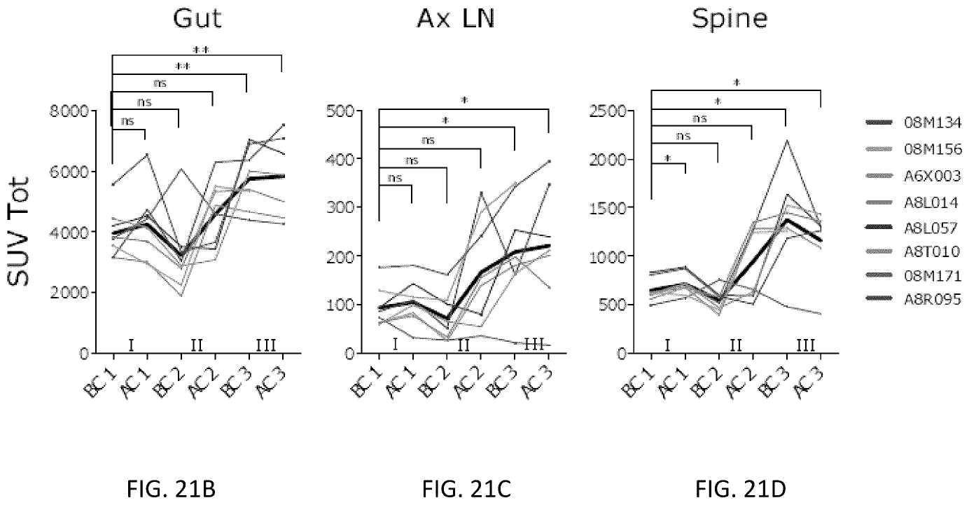

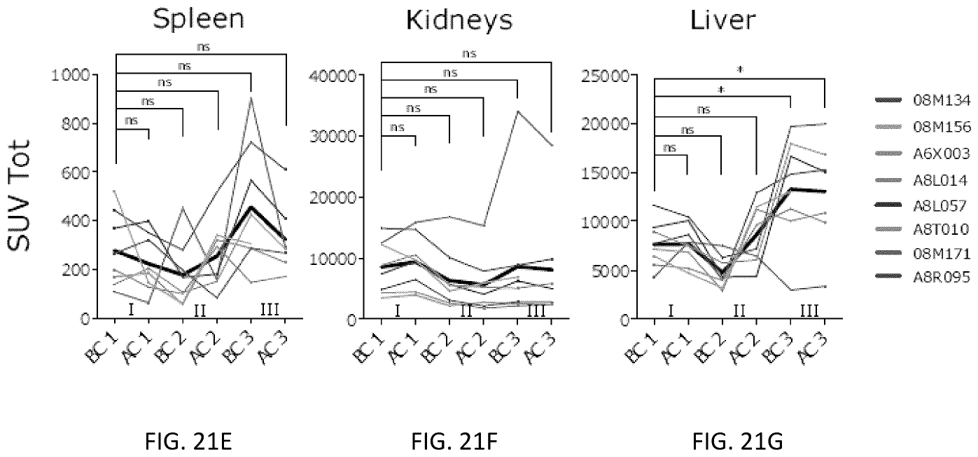

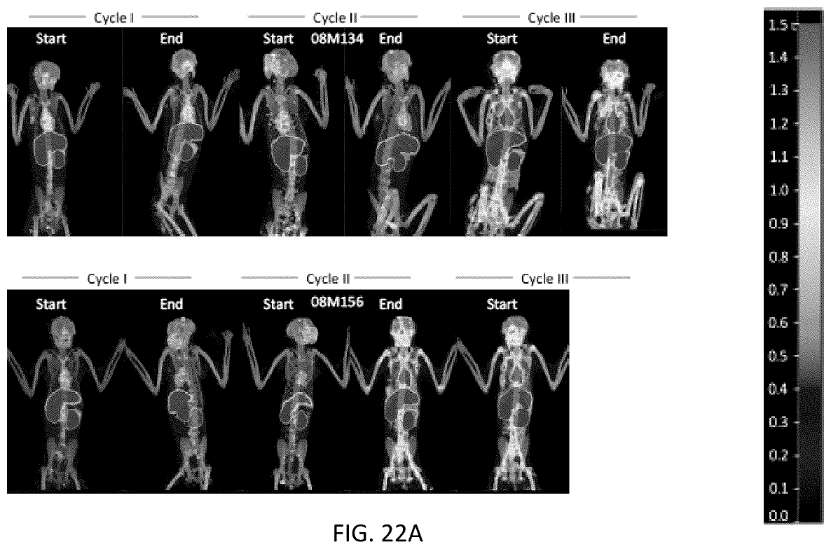

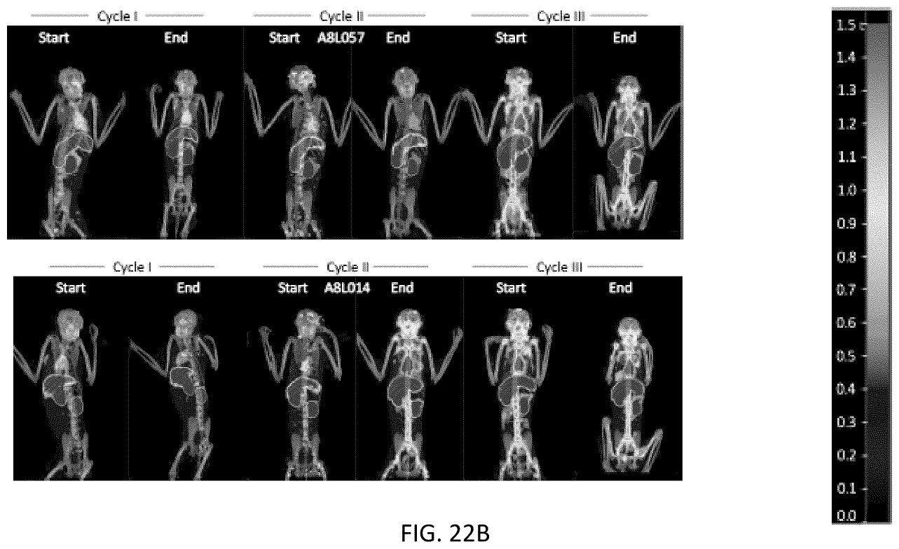

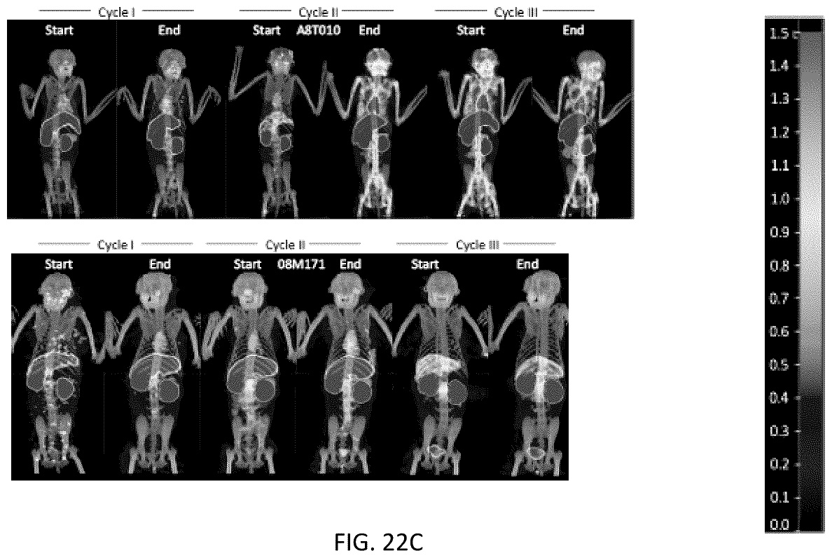

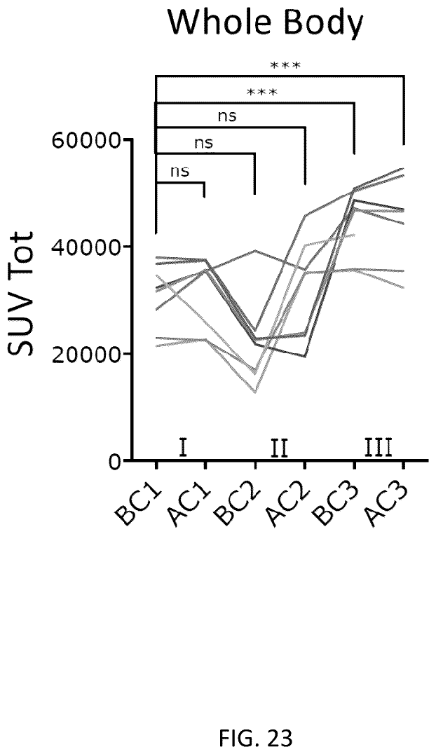

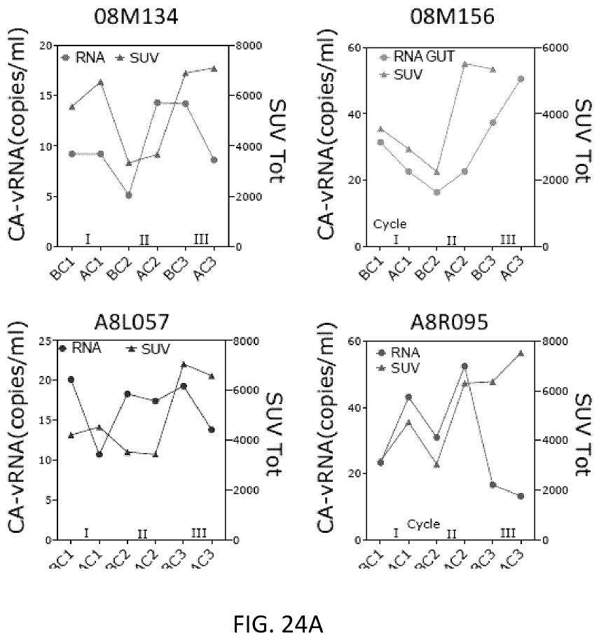

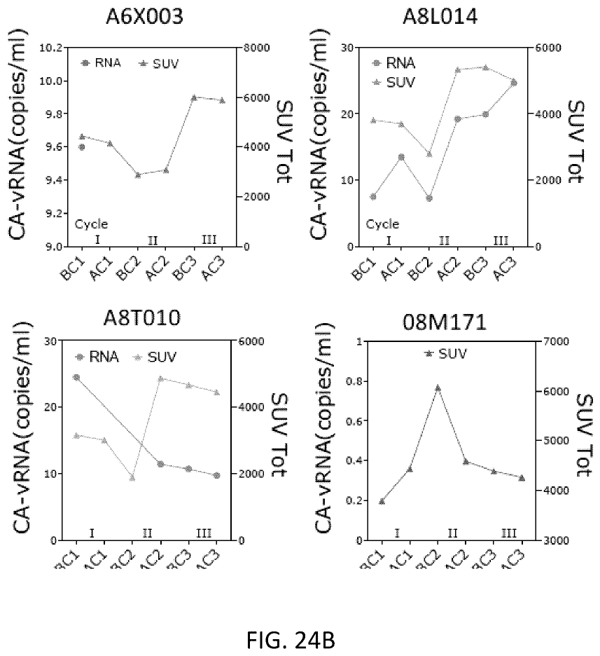

Viral reactivation was documented also using immunoPET/CT. The ⁶⁴Cu-anti-gp120 Fab2(7D3) probe was injected 24 hrs before and after, respectively, the beginning and the end of the first 3 galunisertib cycles and scans performed before and after the first and the last dose. As shown in FIG. 21 and FIG. 22, the PET signal visibly increased in different areas after cycle 2 (157 in A8R095, 08M156, A8L014 and A8T010) or at the beginning of cycle 3 (in A6X003, 08M134 and A8L057). In 08M171 we observed an increase in the gut area only at the beginning of cycle 2. A corresponding increase in standard uptake values (SUV) was detected in the gastrointestinal area and axillary lymph nodes (FIGS. 21B-21C) and was significant in cycle 3 compared to before cycle 1 (BC1). In these anatomical areas, SUV increases correspond to increases in viral replication as shown in previous studies (62, 110). A PET signal increase was also noted in the area of the vertebral column (spine). Spine SUV already increased significantly with the first cycle (FIGS. 21B-21C). However, neither cerebrospinal fluid (CSF) nor bone marrow (BM) was collected during the study, and we have no prior validation of the specificity of the signal in the spine. Hence, whether this signal corresponds to increased viral replication and whether this occurs in the vertebral bones or cerebrospinal fluids remains to be determined. No SUV increase was present at the level of the spleen or kidney, where probe accumulation and background signal likely masked any specific signal. However, a significant increase in SUV was detected in the liver (FIGS. 2B-2C) and whole-body (FIG. 23). Of note, the SUV increase in the gut area in most cases followed an increase in cell-associated vRNA detected in colorectal biopsies at the same time points during treatment (FIGS. 24A-24B).

### Example 7—a Four Cycle Treatment with Galunisertib Decreases Viral Reservoir in Absence of Systemic Inflammation

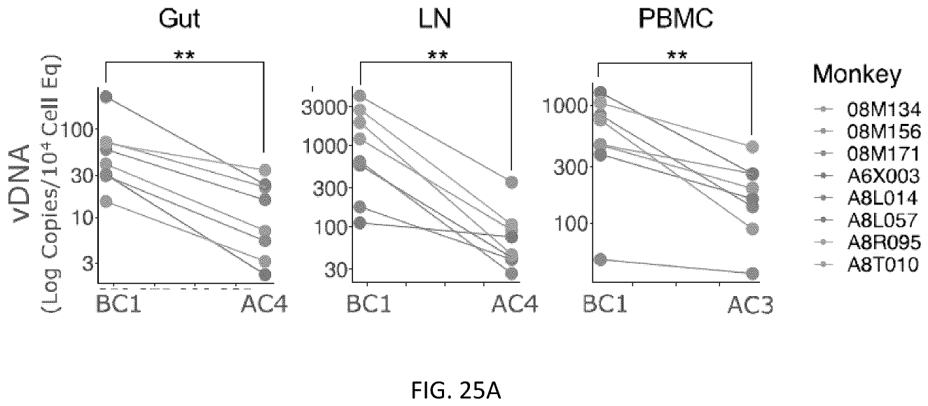

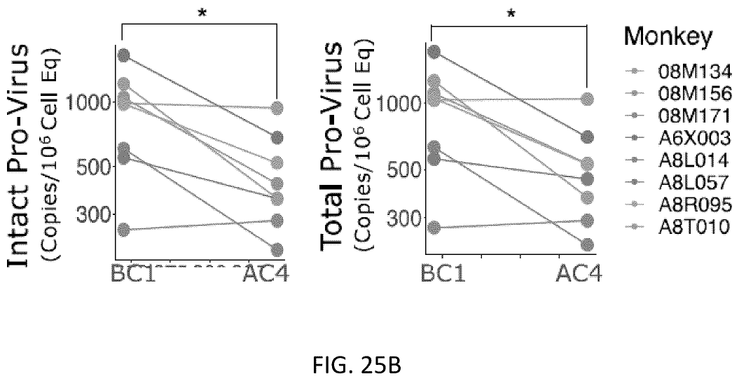

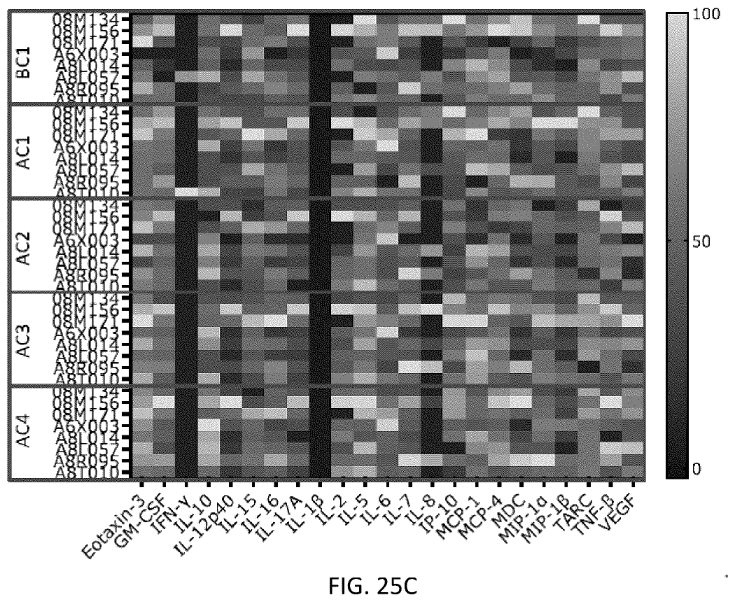

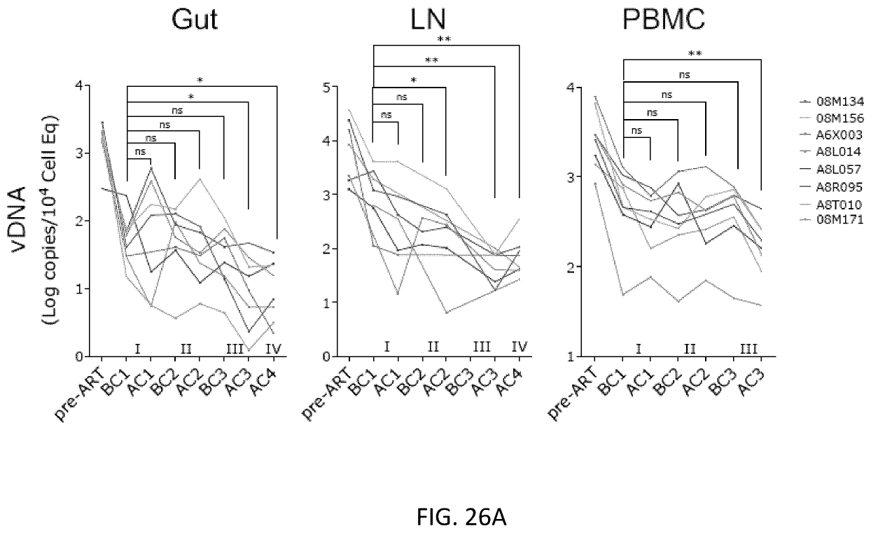

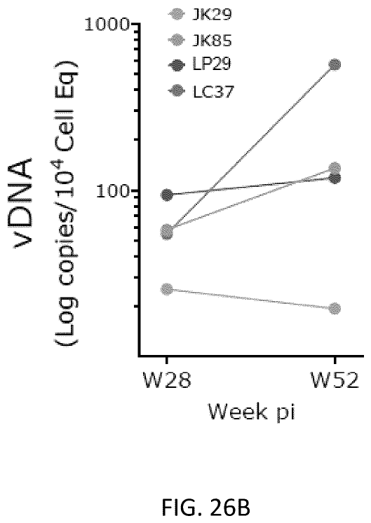

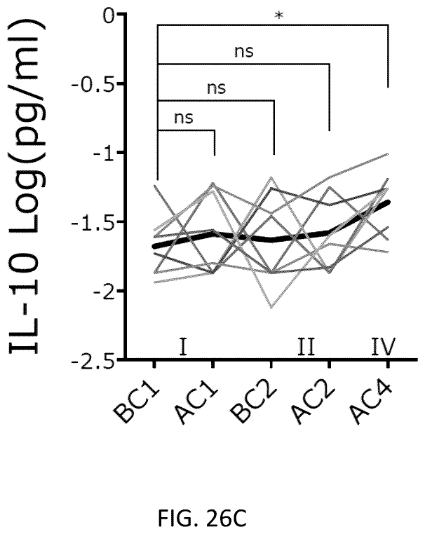

To determine the impact of galunsiertib on SIV reservoir, CA-vDNA was measured in PBMC, colorectal biopsies and lymph nodes (LN). A significant decrease in CA-vDNA was detected in all tissues between week 35 pi (beginning of cycle 1, BC1) and week 49 pi (after/end of cycle 4, AC4) for gut and LN, and between week 35 pi (BC1) and end of cycle 3 (AC3) for PBMC (FIG. 25A, AC4 not measured for PBMC and FIG. 26A). In the gut and LN (right axillary), decreases range from a Log to ⅓ of a Log (gut: median 0.77; range:0.33-0.94 folds LN: median: 0.93; range:0.33-0.98 folds decrease). In the PBMC the decrease was slightly less pronounced with a median half Log decrease (183 median: 0.58; range 0.28-0.88 folds decrease). However, the comparison for the PBMCs was between week ˜35 and week ˜45 (end of cycle 3, AC3) instead of the end of all 4 cycles, because a snap frozen pellet was not available at the end of cycle 4 for PBMCs. The SIV reservoir in PBMCs was also monitored by SIV-IPDA comparing before cycle 1 (BC1) to the end of cycle 4 (AC4). Of note, we observed significant decreases of both total and intact provirus by SIV-IPDA. Intact provirus declined similarly to the CA-vDNA with a median of half Log (median: 0.53; range:0-0.71 folds decrease).

In contrast, no decline in CA-vDNA was detected in the PBMCs of a group of 4 macaques infected intravenously with the same stock of SIVmac239M2 for a separate study. These 4 macaques were placed on ART on week 6 pi as in our study. However, no decline in CA-vDNA was detected under ART, between weeks 28 and 52 pi (untreated group, FIG. 26B). Thus, we conclude that the decline in CA-vDNA in PBMC was not due to ART-alone.

No significant changes we found in any of the clinical variables (chemistry and hematology, data not shown) measured before, during and after the galunisertib therapy. Moreover, we observed no changes in the concentrations of inflammatory chemokines and cytokines measured in plasma before and after the first 2 treatment cycles and at the end of the last cycle (FIG. 25C). The only difference in cytokine and chemokine levels after galunisertib 200 treatment was a small increase in IL-10 detected at the end of the last cycle compared to before treatment (FIG. 26C).

### Example 8—Galunisertib Treatment Drives an Effector Phenotype in T and NK Cells

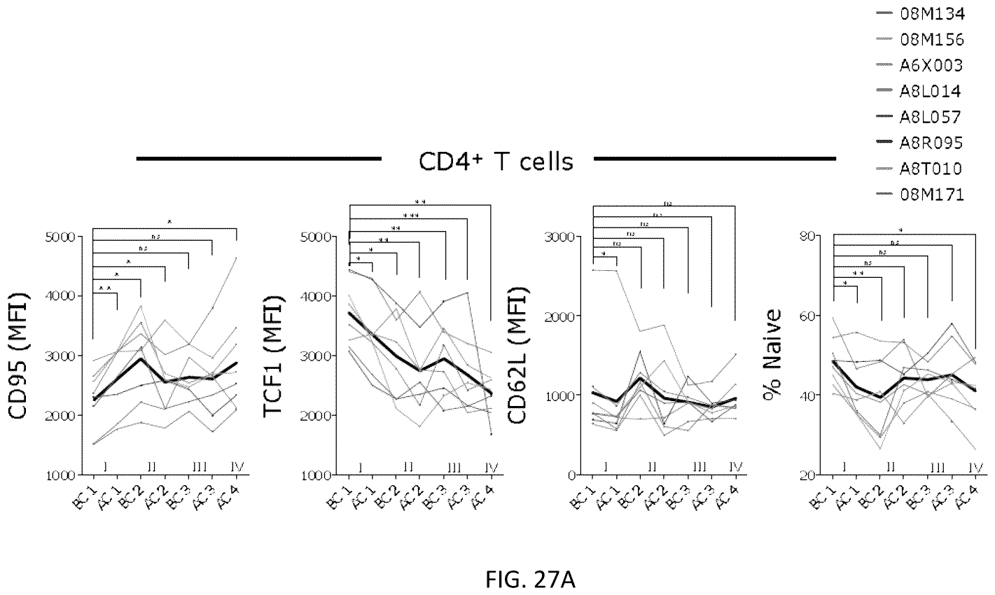

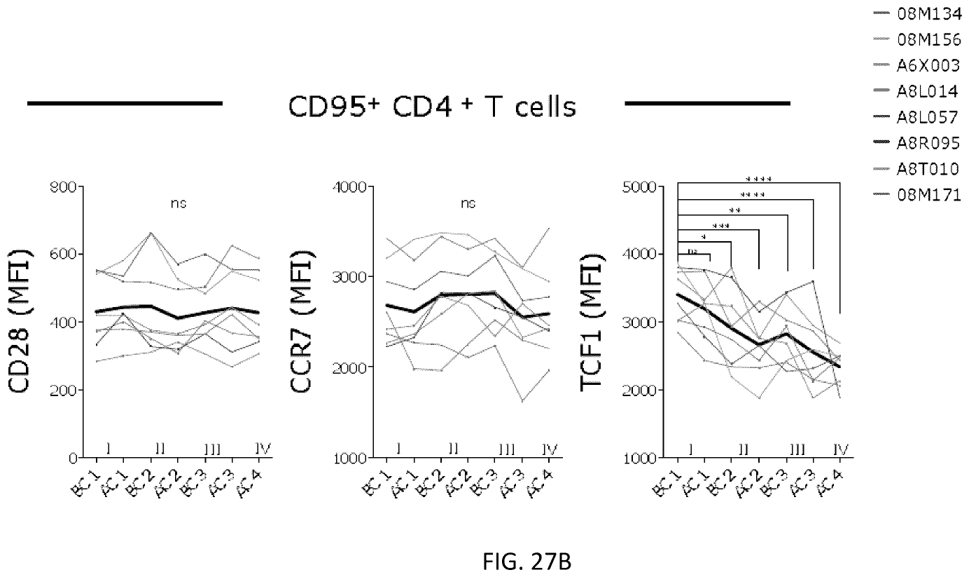

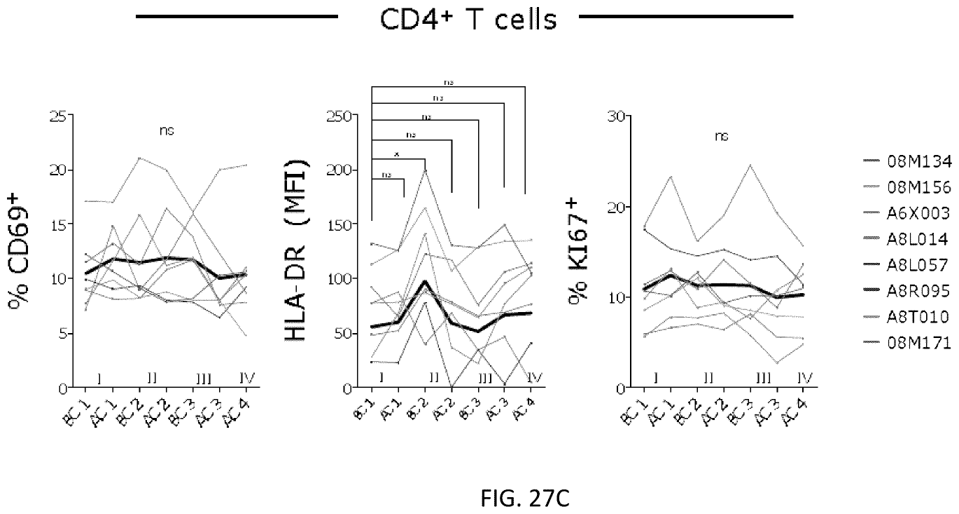

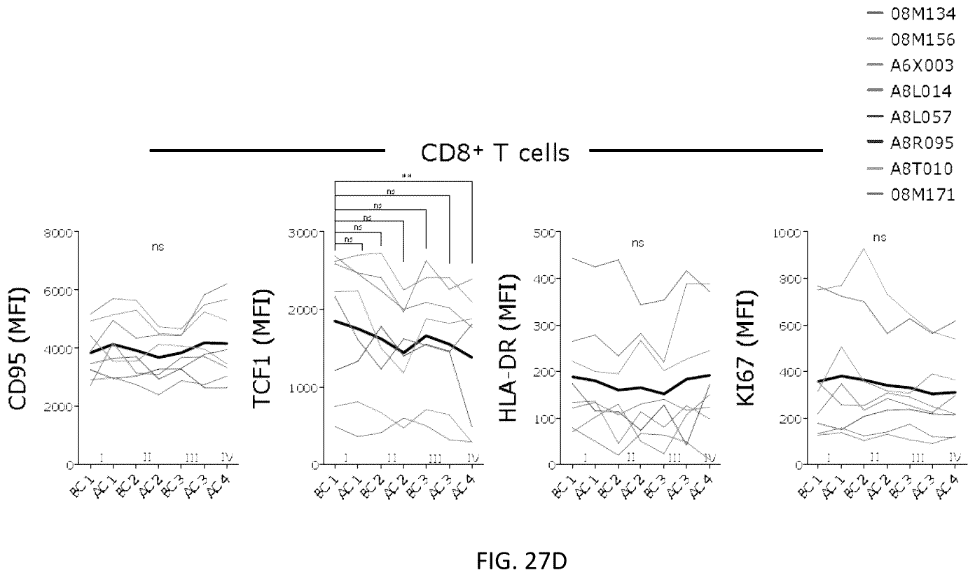

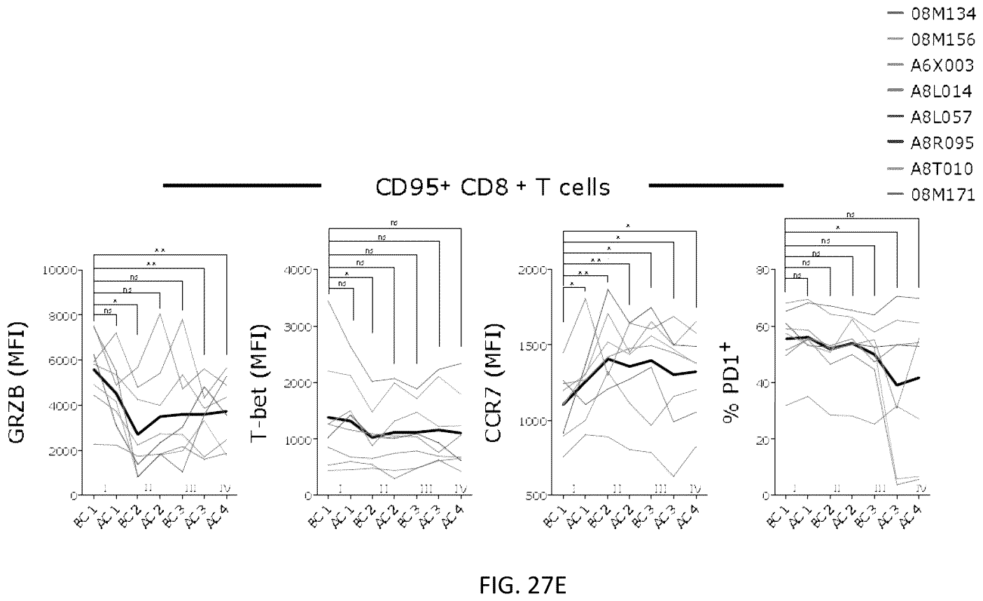

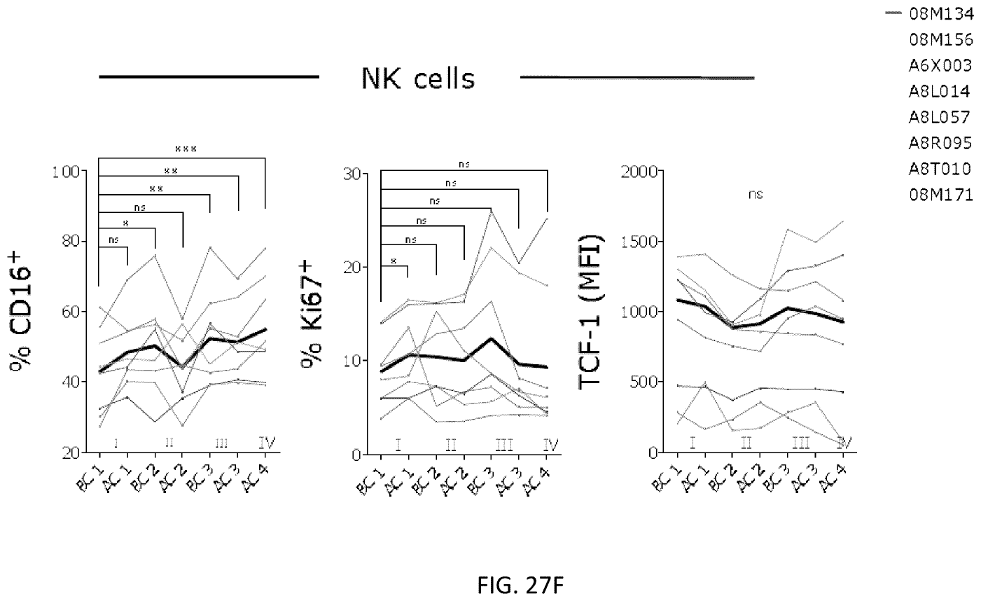

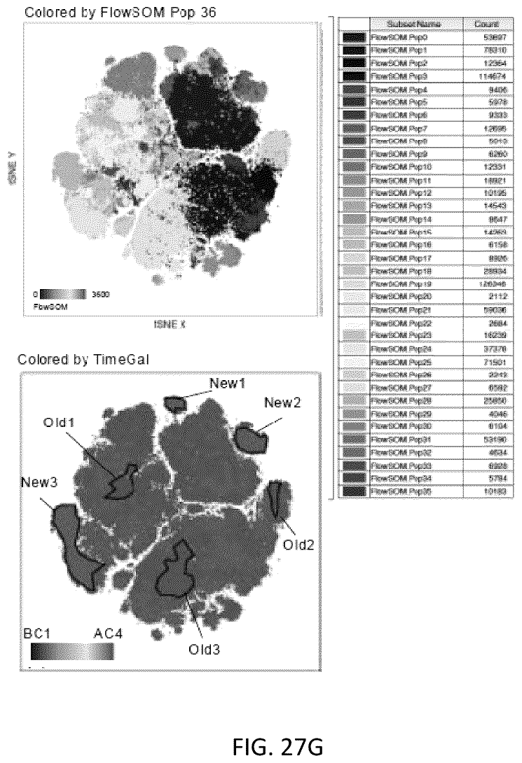

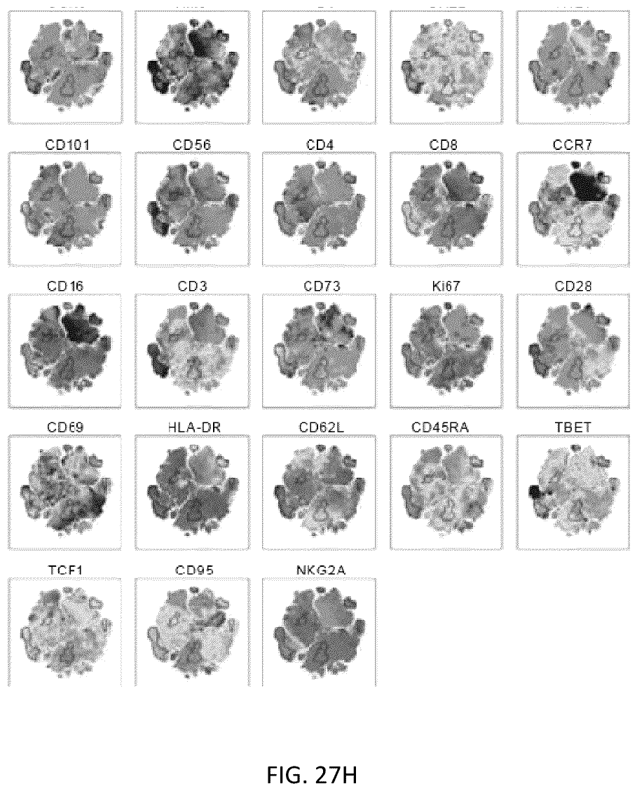

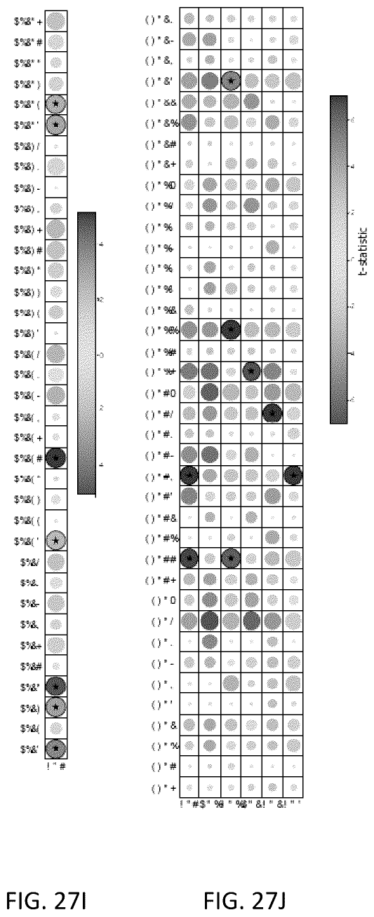

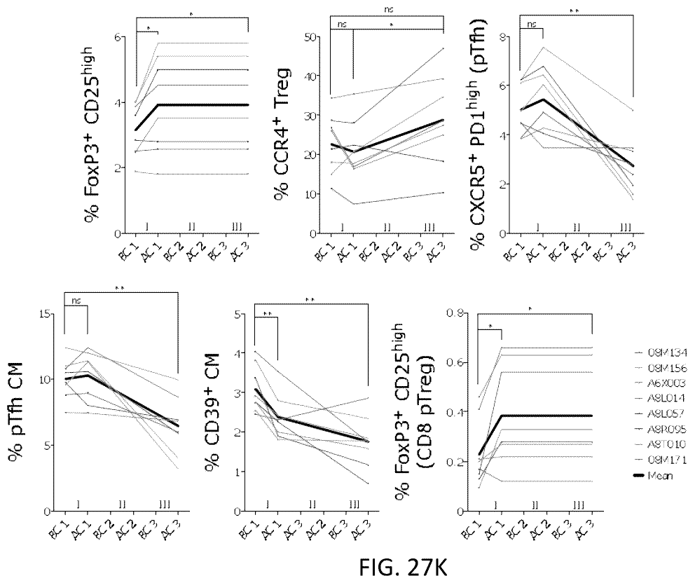

The phenotype of PBMCs before and after Galunisertib treatment was monitored by high parameter flow cytometry of T and NK cell subsets and phenotype (Table 4). Classical subsets and single-color analysis of MFI revealed a substantial increase in the expression of CD95 and a profound consistent decrease in TCF1 expression in CD4+ T cells that continued throughout the treatment (FIG. 27A).

TABLE 4
List of Antibodies used for phenotyping.

| | Marker | Clone | Maker |
|---|---|---|---|
| PBMC High-Dim | | | |
| DAPI | FVS440UV | Live Dead | BD |
| BUV395 | CD4 | L200 | BD |
| BUV563 | CD8 | RPA-T8 | BD |
| BUV661 | CCR7 | 3D12 | BD |
| BUV737 | CD16 | 3G8 | BD |
| BUV805 | CD3 | SP34-2 | BD |
| BV480 | Ki67** | B56 | BD |
| BV510 | CD28 | 28.2 | Biolegend |
| BV605 | CD69 | FN50 | Biolegend |
| BV650 | HLA-DR | L243 | BD |
| BV711 | CD62L | SK11 | BD |
| BV750 | CD45RA | 5H9 | BD |
| BV786 | Tbet | 4B10 | Biolegend |
| AF488 | CCR8 | G034E3 | Biolegend |
| BB630 | Tim-3 | 7D3 | BD |
| BB700/PCPeFluo710 | PD-1 | eBioJ105 | eBioscience |
| BB790 | GRZB** | GB11 | BD |
| PE | TCF-1** | S33-966 | BD |
| PE-CF594 | CD95 | DX2 | Biolegend |
| PE-Vio770 | NKG2A | REA110 | Mylteny |
| APC | a4b7 | Act-1 | NHP |
| AF700/R718 | CD101 | V7.1 | BD |
| APC-Fire750 | CD56 | HCD56 | Biolegend |
| Treg/Tfh Panel | | | |
| FITC | FOXP3 | PCH101 | EBioscience |
| PCP-Cy5.5 | CD4 | L200 | BD |
| BV421 | CTLA4 | BNI3 | Biolegend |
| BV510 | L/D | N/A | BD |
| BV605 | CD95 | DX2 | Biolegend |
| BV650 | PD-1 | EH12.2H7 | Biolegend |
| BV711 | CD39 | A1 | Biolegend |
| BUV395 | CD25 | BC96 | BD |
| BUV737 | CD28 | CD28.2 | BD |
| BUV805 | CD8 | RPA-T8 | Biolegend |
| PE-Cy5 | CXCR5 | 398.4A | Biolegend |
| PE-Cy7 | CCR4 | L291H4 | Biolegend |
| APC | TCF-1 | s33-966(RUO) | BD |
| AL700 | CD3 | SP34-2 | BD |

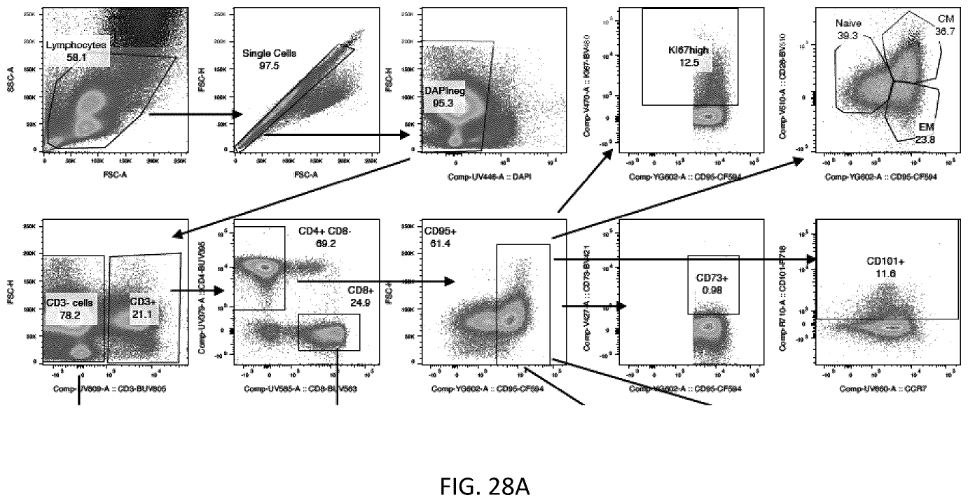

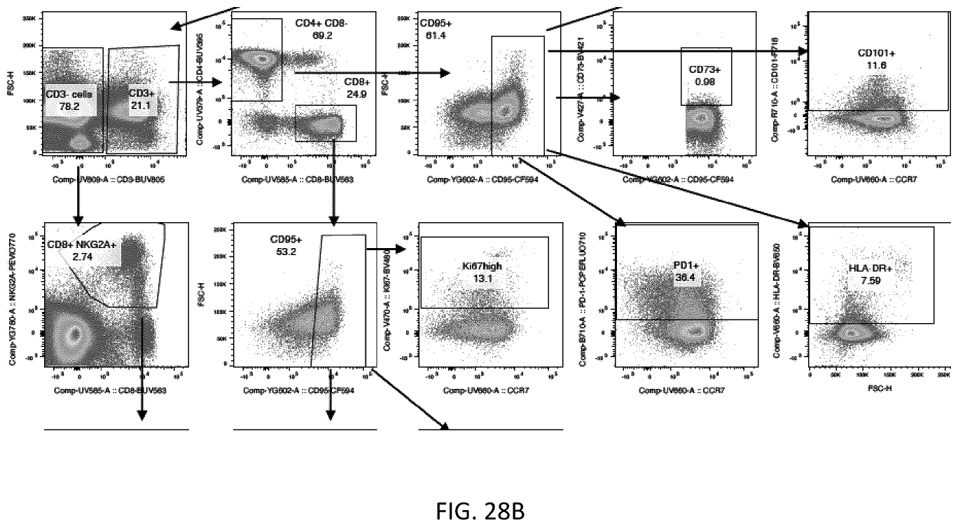

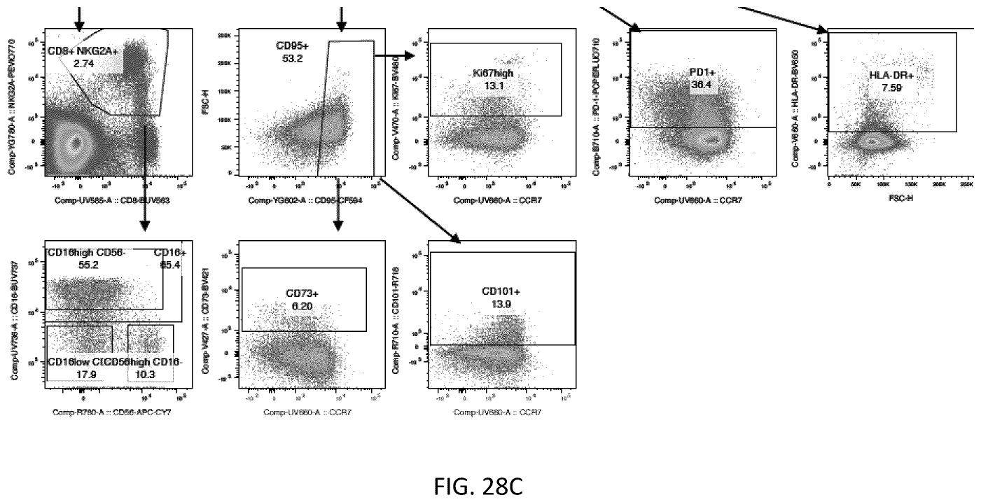

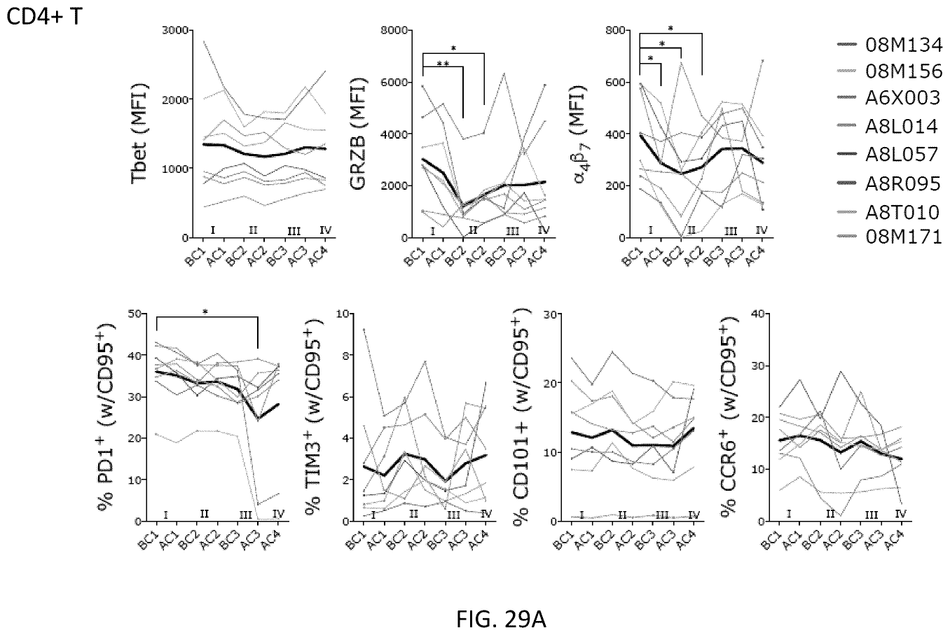

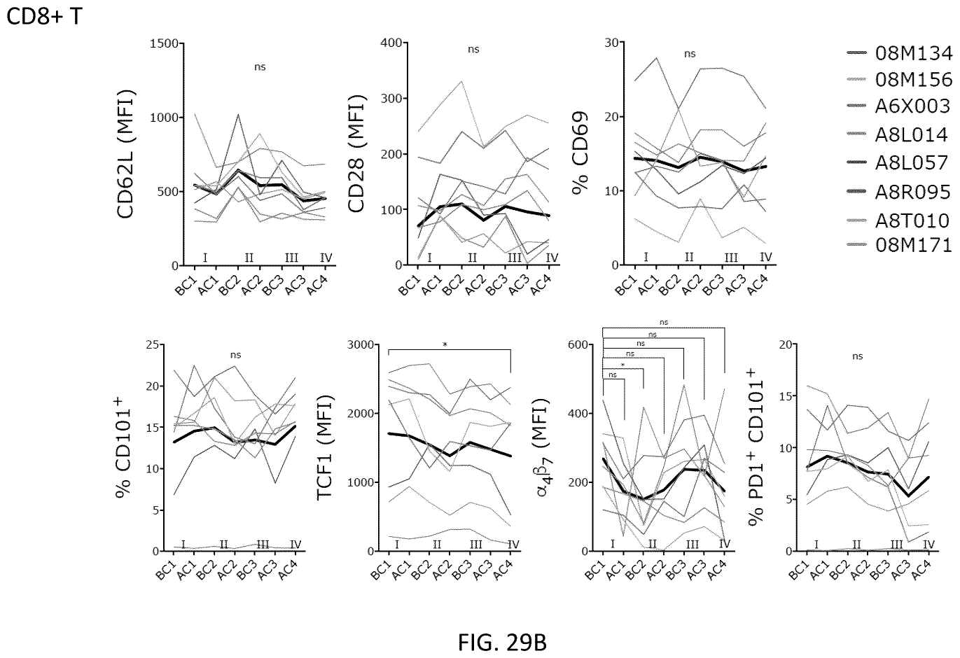

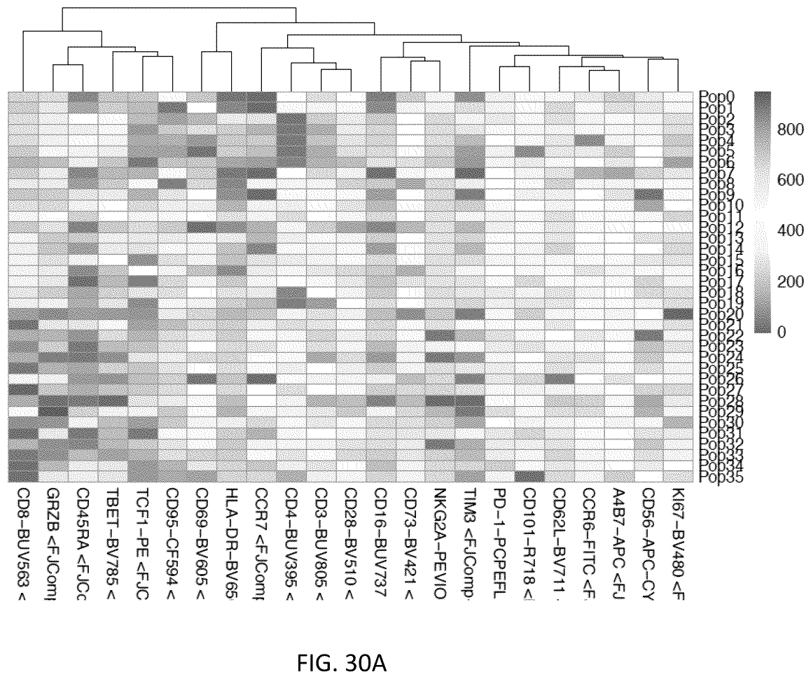

In contrast, a small decrease in CD62L after the first cycle, reverted to baseline during the following cycles. The frequency of naïve cells, defined as CD95− within CD4+ T cells, decreased in parallel with the increase in CD95 (gating strategy 210 in FIG. 28). Interestingly, there was no change in the expression of CCR7 or CD28 within CD95+CD4+ T cells (FIG. 27B). Hence, the frequency of central memory and effector memory as defined by CD95 and CD28 or CCR7 did not change (data not shown). The levels of T-bet did not change significantly (FIG. 29A). However, we detected a downregulation of the gut homing receptor integrin α4β7 and a decrease in the levels of granzyme B (GRZB, FIG. 29A). Of note, the expression of activation markers CD69, HLA-DR and Ki67 remained mostly unchanged, with the exception of an increase in HLA-DR at the beginning of cycle 2 compared to before galunisertib (FIG. 27C). Interestingly, the effect of Galunisertib on CD8+ T cells was not as pronounced as it was on CD4+ T cells. No significant increase was detected in CD95 expression and TCF1downregulation reached significance only at the end of the treatment (FIG. 27D). Markers of cell activation like HLA-DR, Ki67 and CD69 did not change (FIG. 27D and FIG. 29B). However, a significant decrease in T-Bet at the beginning of cycle 2 (BC2) was detected compared to before treatment (BC1). Finally, in contrast to CD4+ T cells, the decrease in GRZB was more pronounced at several time points during treatment and there was a sustained significant increase in CCR7 expression in memory CD8+ T cells (FIG. 27E). Interestingly, the expression of PD1 on both CD4+ T cells and CD8+ T cells either did not change or was slightly downregulated. Importantly, the frequency of PD1+CD101+(TCF1low) exhausted memory CD8+ T cells (111) did not change (FIG. 29B). Finally, within NKG2A+CD8+NK cells, there was a pronounced increase in CD16 expression and an initial increase in the proliferation marker Ki67 (AC1 vs BC1; FIG. 27F).

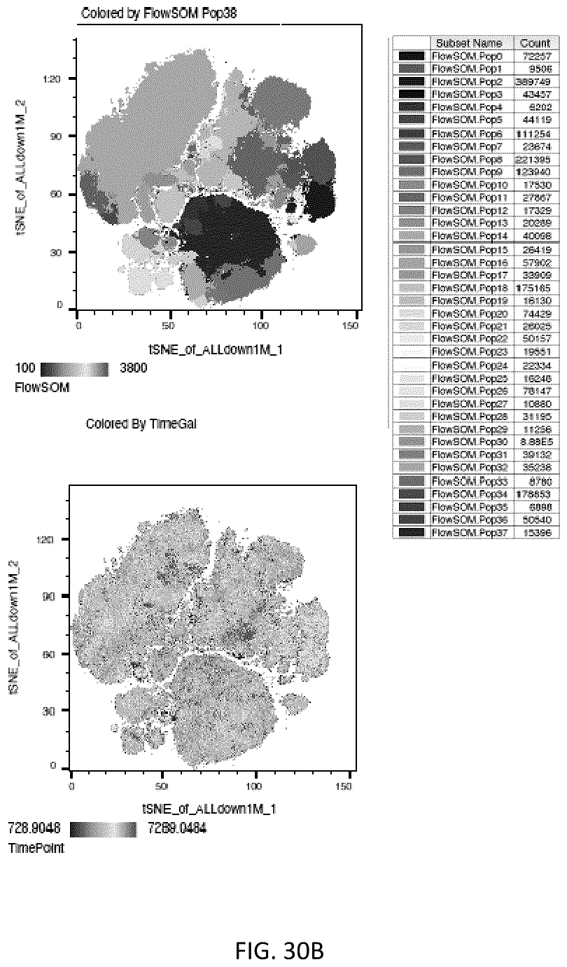

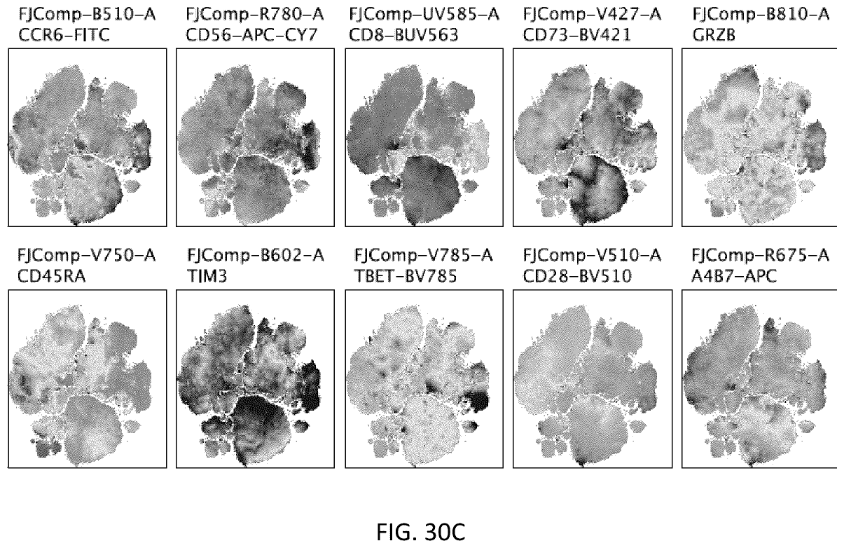

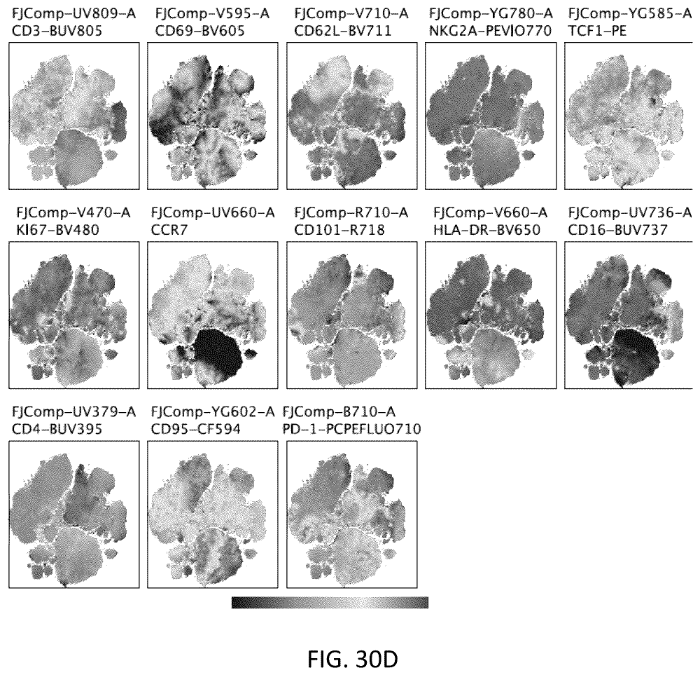

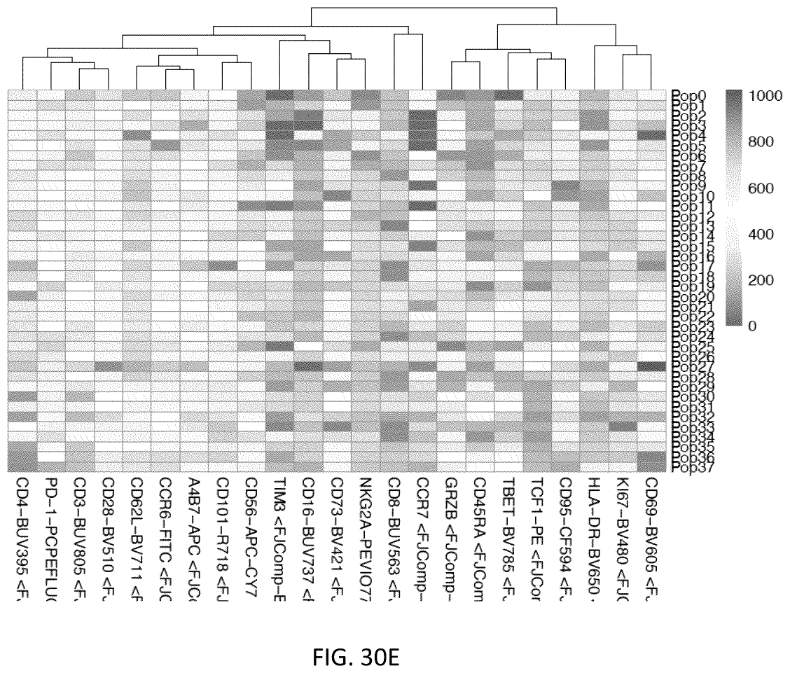

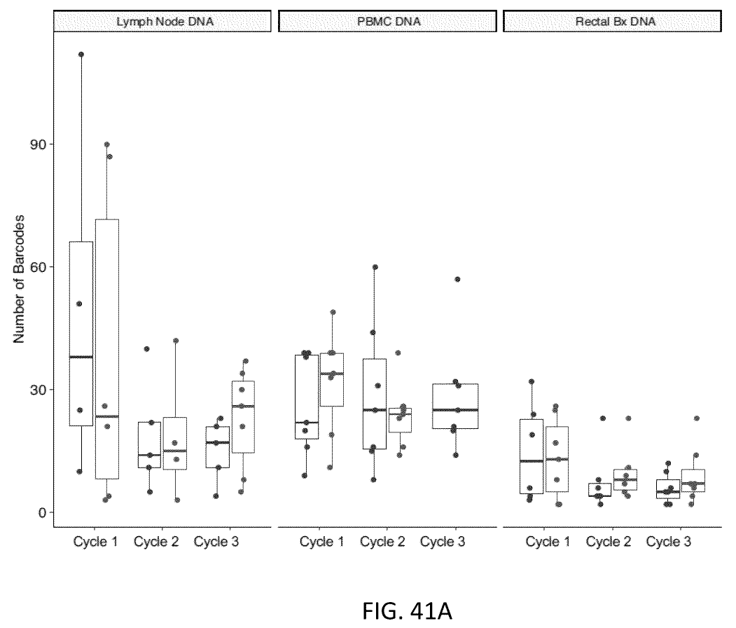

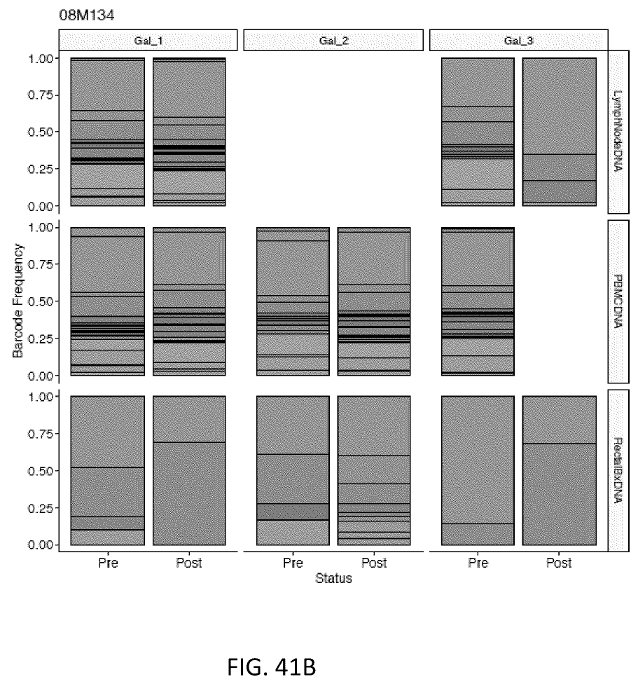

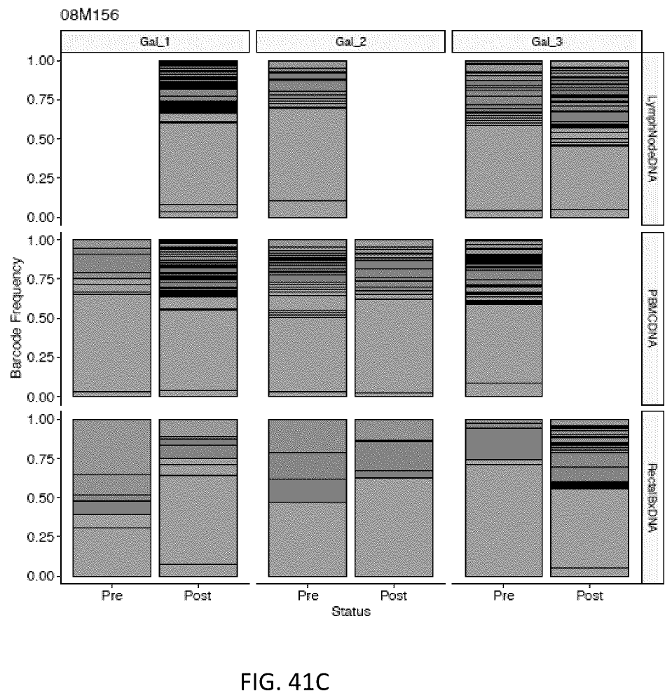

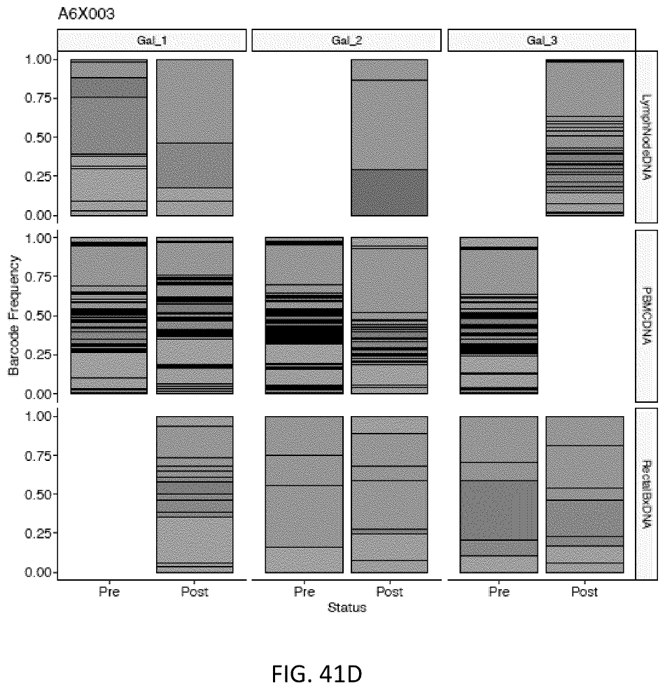

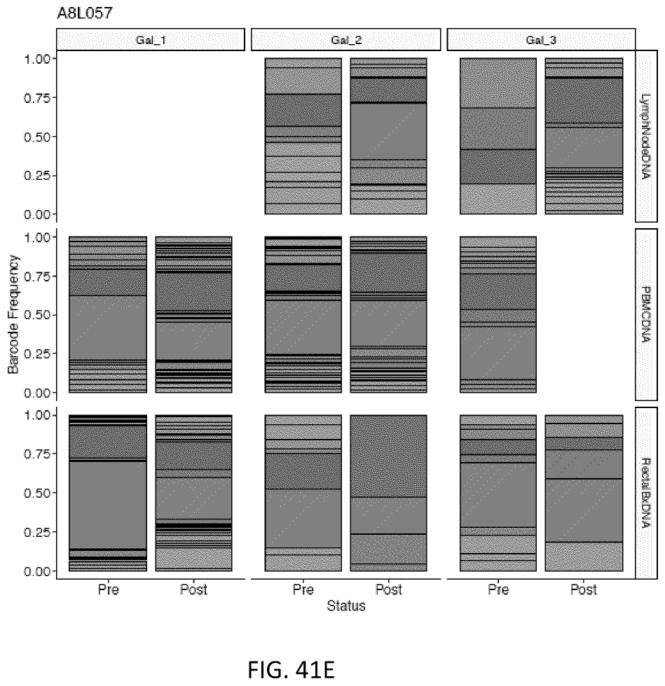

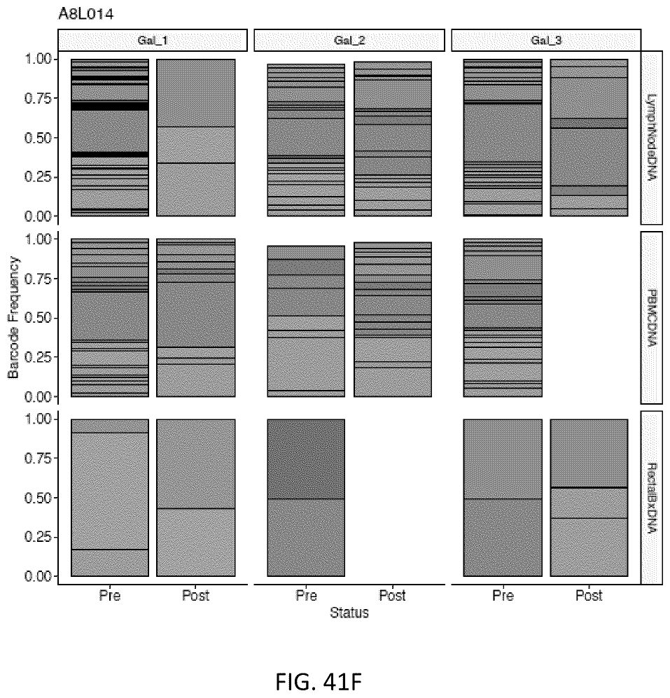

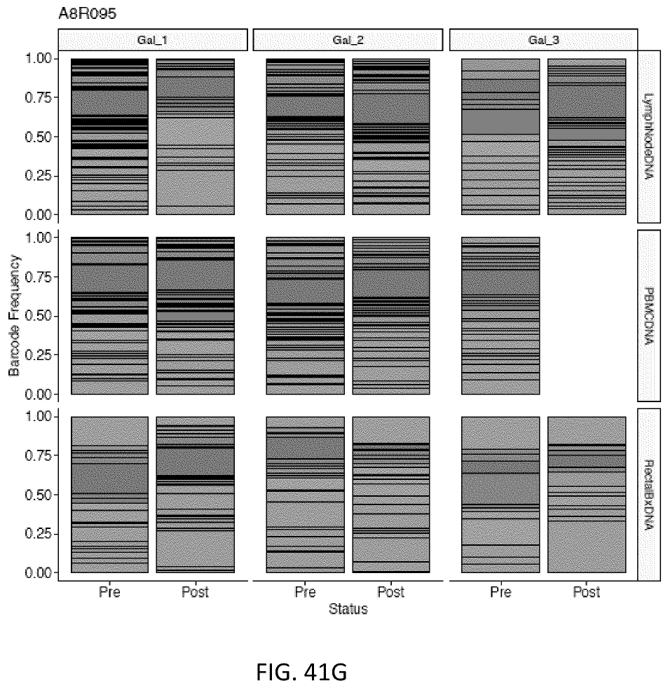

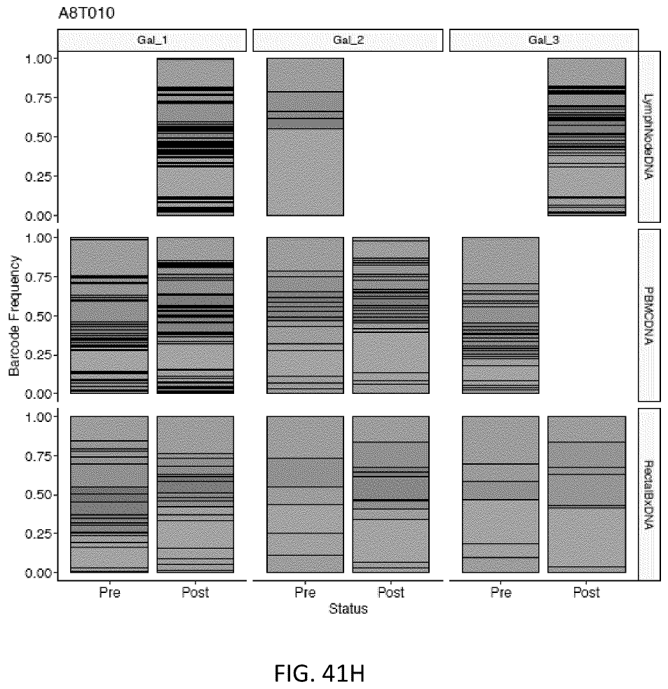

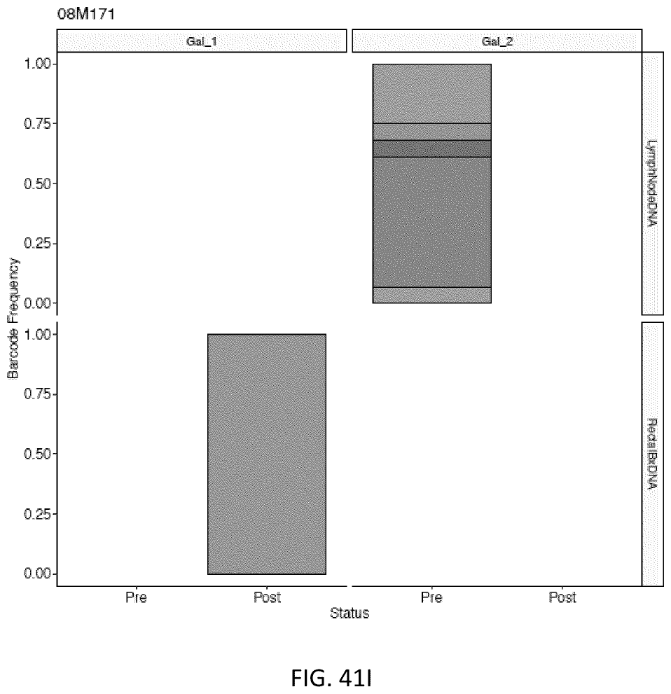

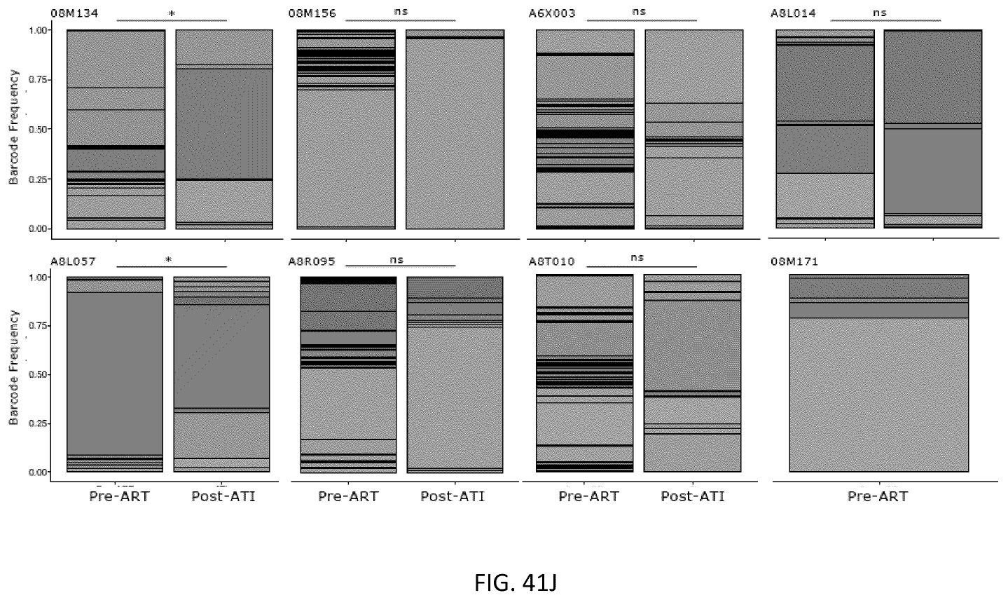

High-dimensional data visualization with tSNE and clustering analysis with FlowSOM (112) confirmed the results of the classical analysis. We performed tSNE and FlowSOM after data clean up with FlowClean and normalization with the SwiftReg algorithm (113). The analysis was time points analyzed 235 (FIG. 30B and FIG. 30C). When we compared only BC1 and AC4, the PhenoGraph clustering algorithm identified 36 populations. FlowSOM with 36 populations identified 6 populations of CD4+ T cells, 6 of CD8+ T cells and 4 of NK cells (NKG2Ahigh CD8+; FIG. 30A). The remaining populations were likely monocytes and other minor subsets. Direct comparison of each population revealed a decrease in Pop 31 (naïve CD8 T cells) and Pop 30 (central memory Ki67+CD8+ T cells) and an increase in Pop 2 and 3 (effector and central memory CD4+ T cells). Finally, there was a decrease in HLA-DR high Pop0 and an increase in CD16high NKG2A- Pop14 (FIG. 41 and FIG. 30A). Visual inspection of the tSNE plots (FIG. 27G-FIG. 27H) revealed 3 areas mostly occupied by cells in the post-galunisertib AC4 group (New1, 2 and 3) which were characterized by high levels of CD16 and GRZB (New 3 is likely NK cells). In contrast, 3 areas mostly occupied by cells in the pre-galunisertib group BC1 (Old1, 2 and 3) were characterized by high levels of TCF1 and low CD95, confirming the finding of the classical analysis. Analysis of all time points with phenograph-derived 38 populations in FlowSOM recapitulated findings obtained with the classical analysis and the BC1 AC4 comparison with no additional insights (FIG. 27J)

### Example 9—Galunisertib Treatment in Vivo Increases pTreg while Decreasing pTfh

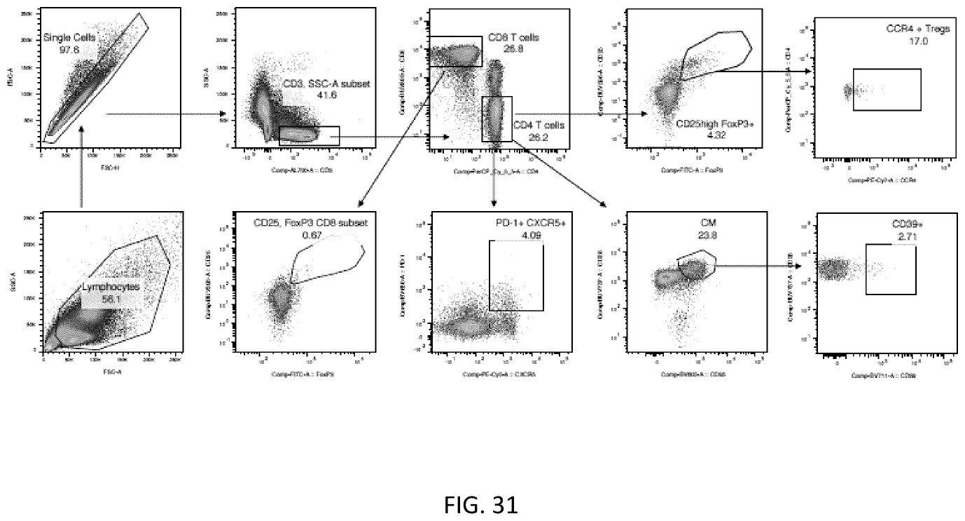

The frequencies of circulating Tregs and Tfh cells were monitored with an established flow cytometry panel (114, 115) after the first and third cycle of galunisertib. The frequency of all CD4+ 253 Tregs (CD25high FoxP3+; gating in FIG. 31) and CD8+ Tregs increased after the first cycle and remained higher until the end of the 3rd cycle, while CCR4+ Treg were proportionally higher at the end of the treatment compared to before (FIG. 27K). In contrast, circulating Tfh (CXCR5+PD1+; gating in Fig S8) were lower at the end of the Galunisertib treatment both within total and central memory CD4+ T cells (FIG. 27K). Finally, the expression of CD39 (ecto-nucleotide triphosphate diphosphohydrolase 1), which tracks within extracellular adenosine and immunosuppressive effects, was lower on total and central memory CD4+ T cells (FIG. 27K).

### Example 10—Bulk RNAseq of PBMC and Single-Cell (Sc)RNAseq Analysis of LN Confirm a Profound Shift Toward Effector Phenotype

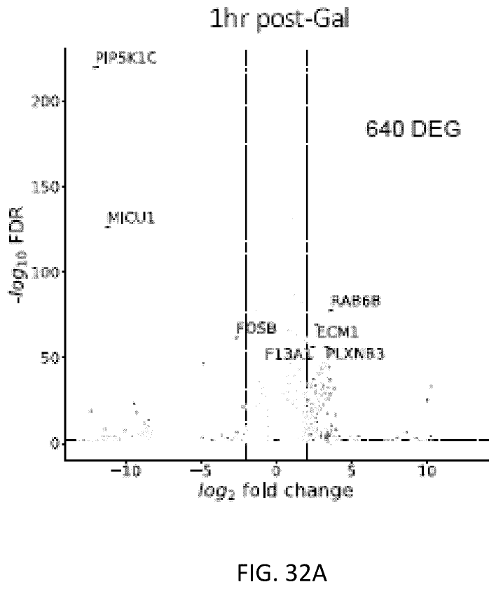

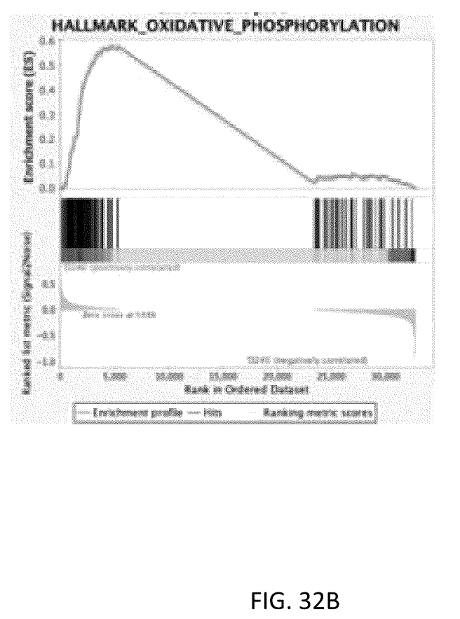

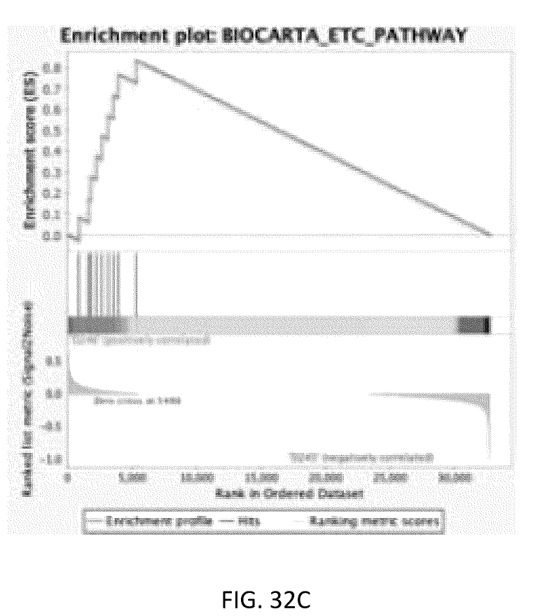

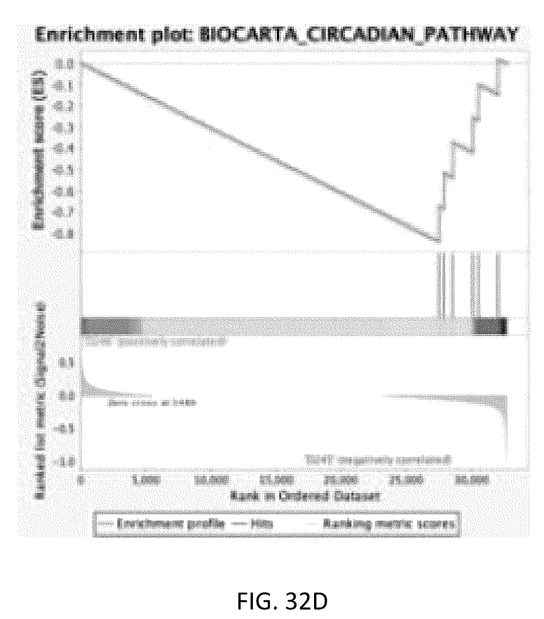

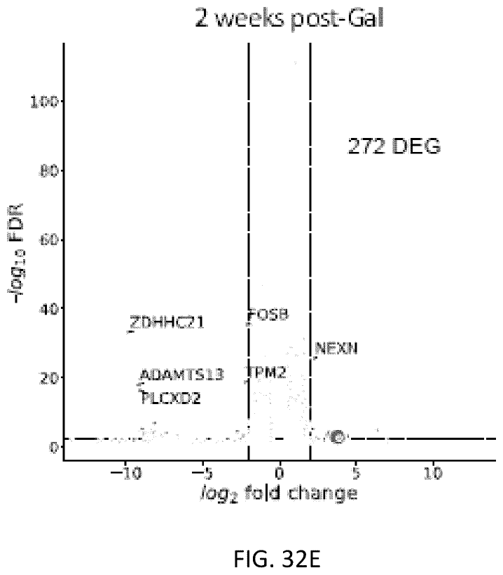

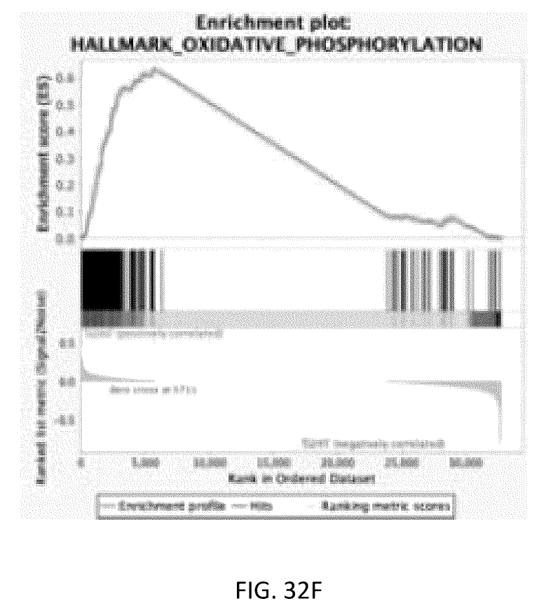

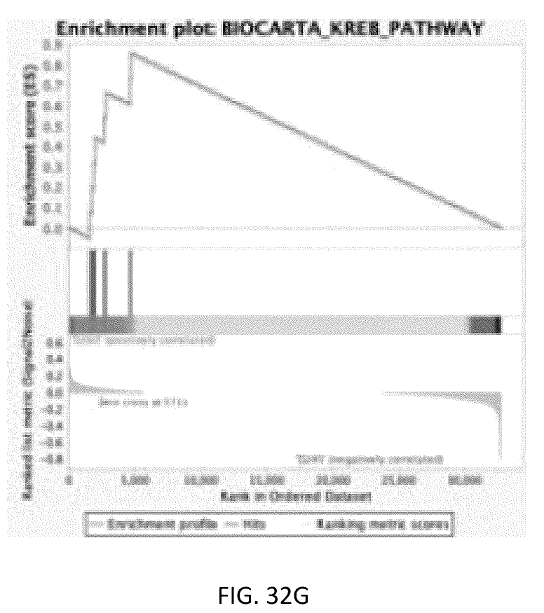

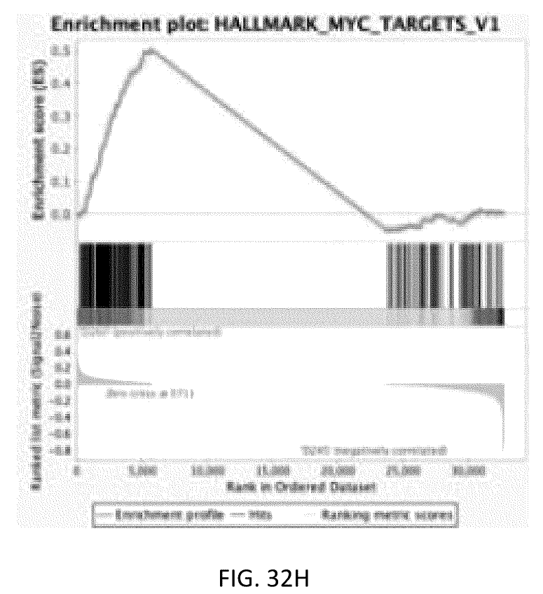

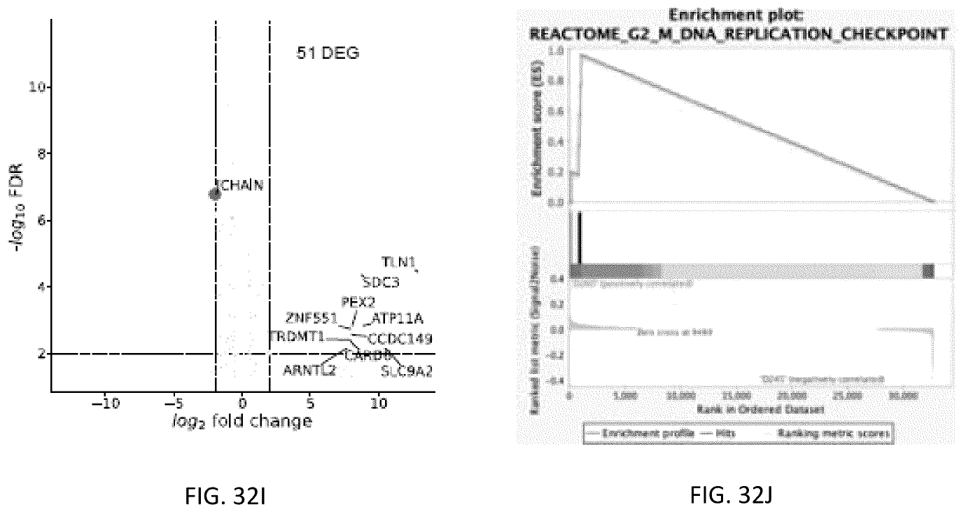

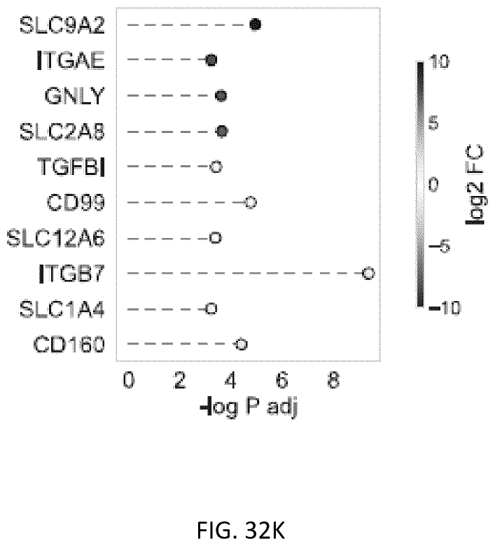

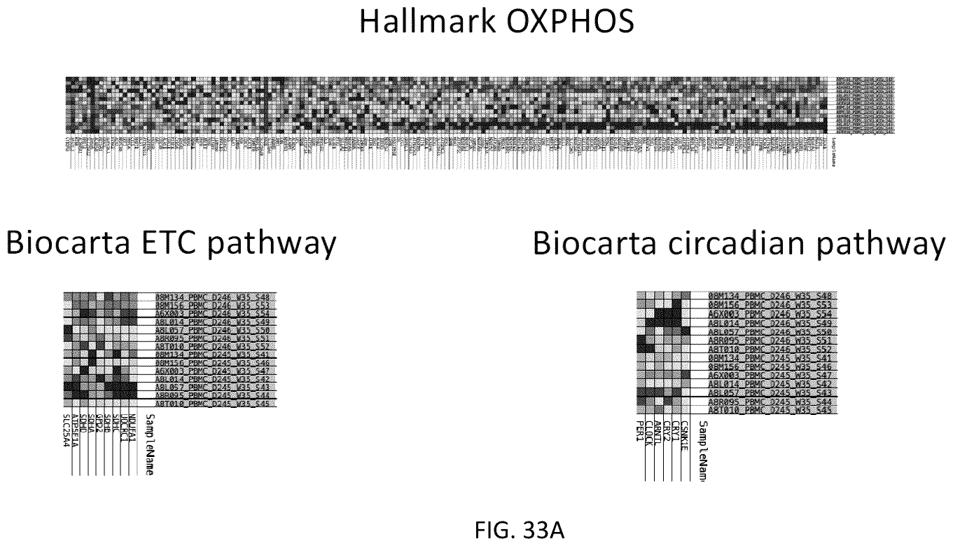

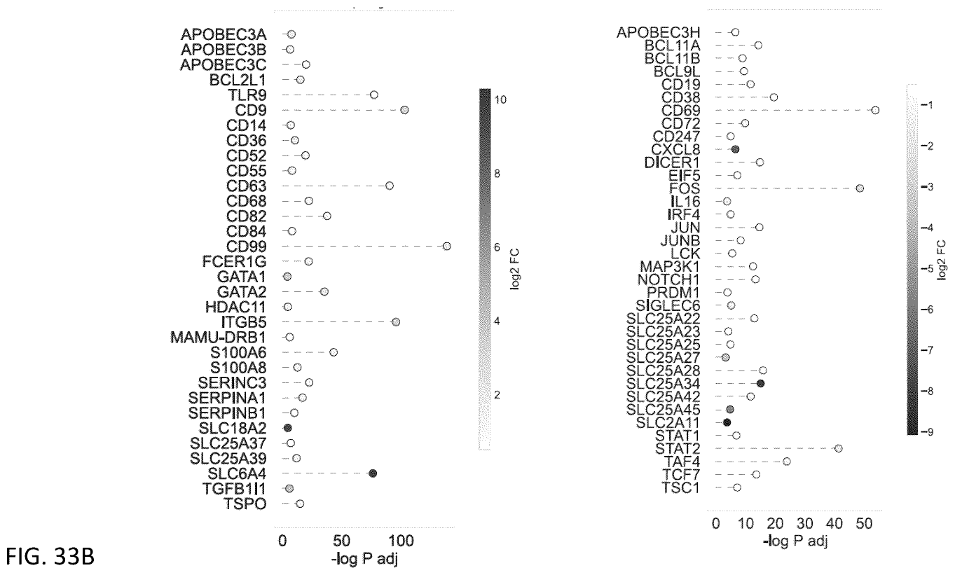

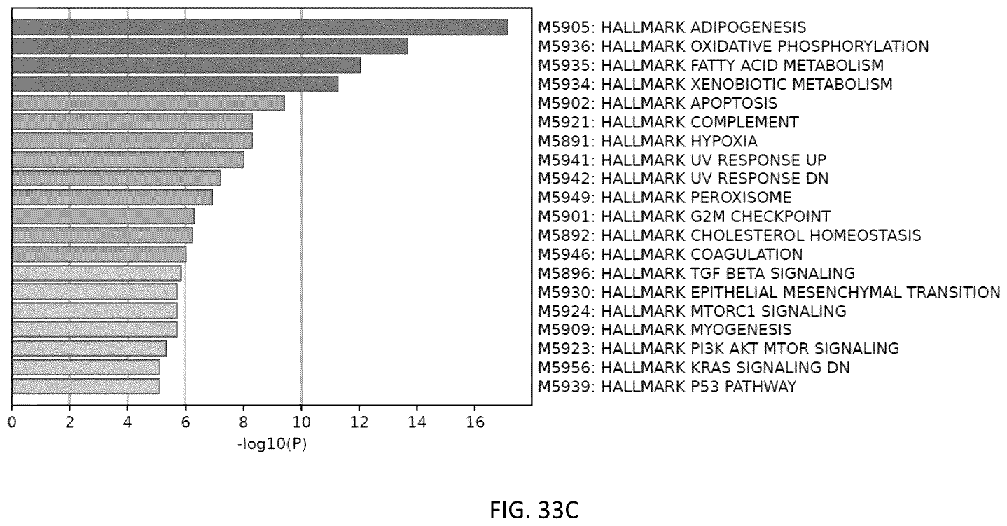

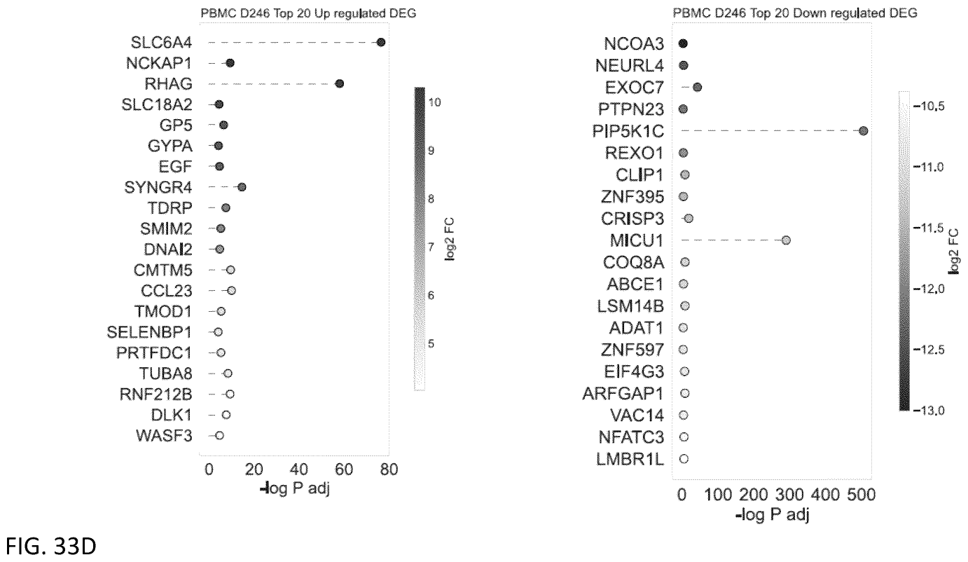

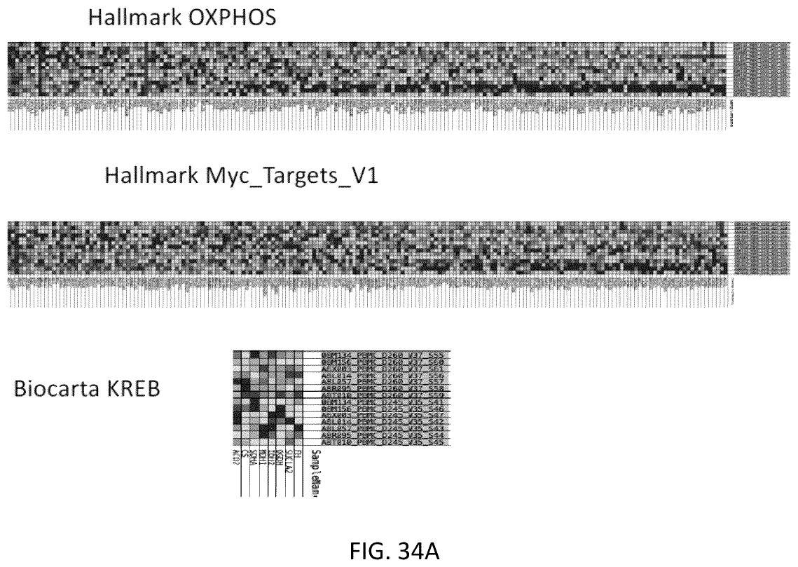

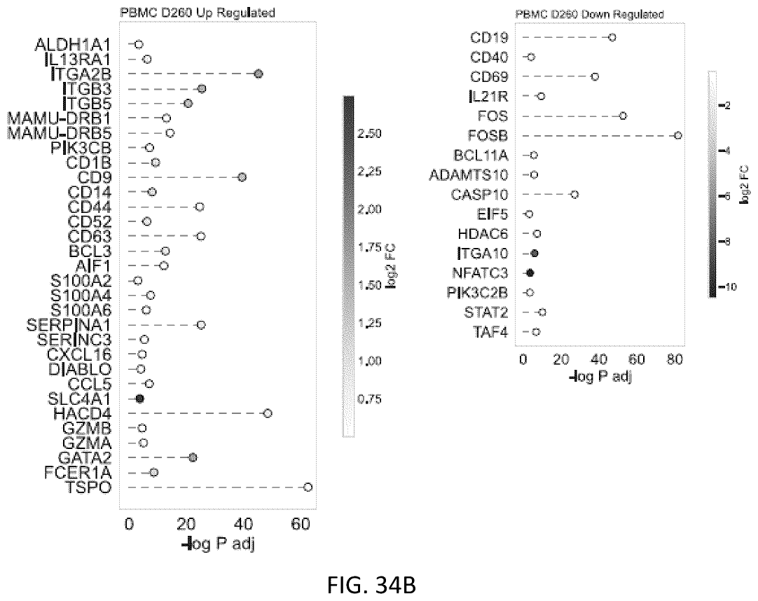

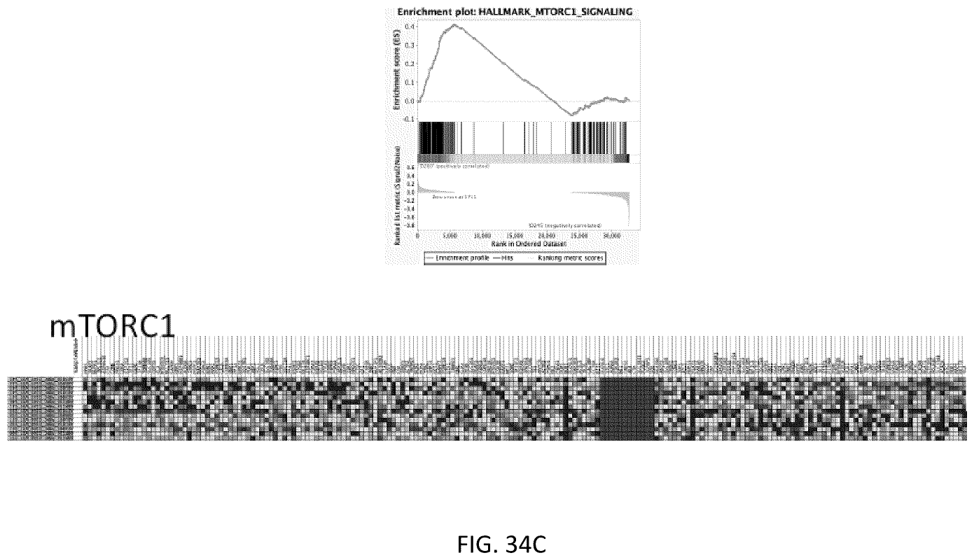

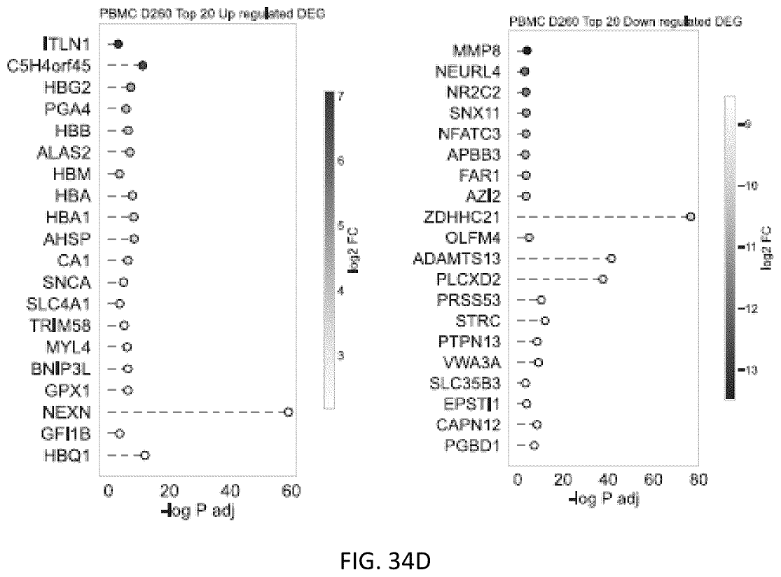

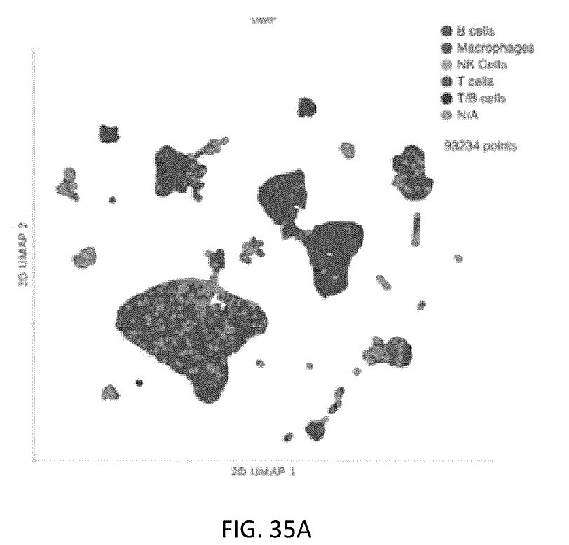

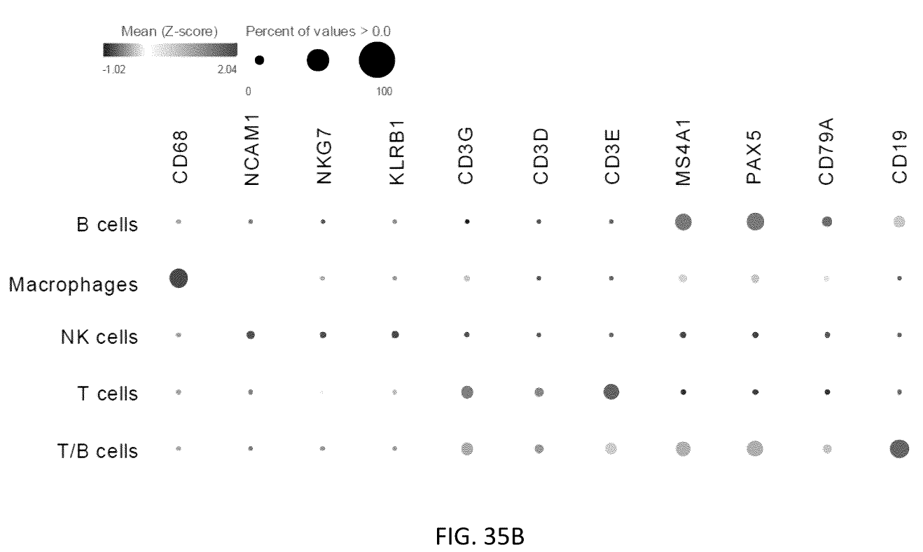

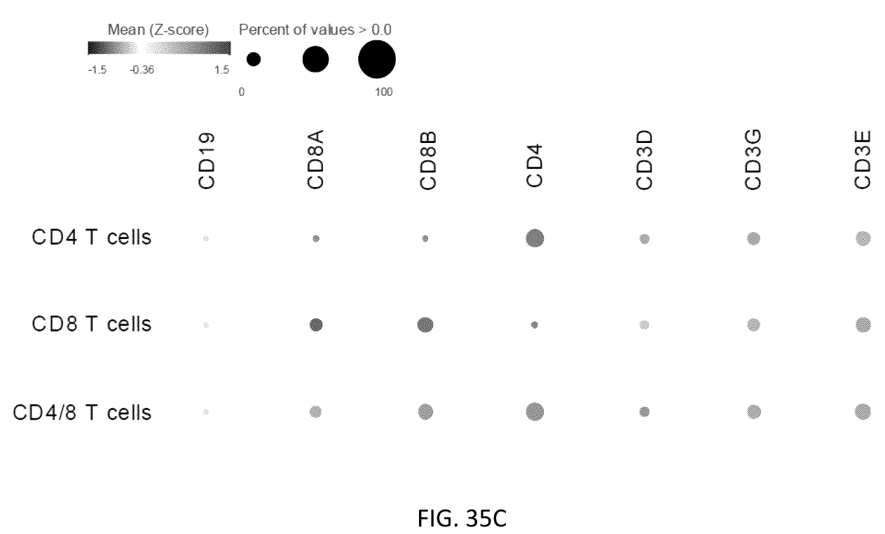

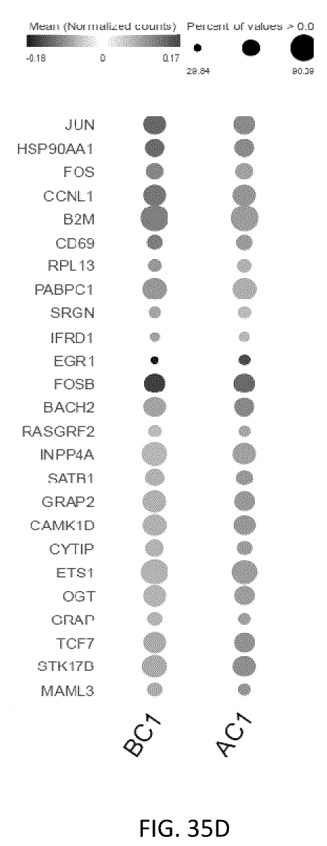

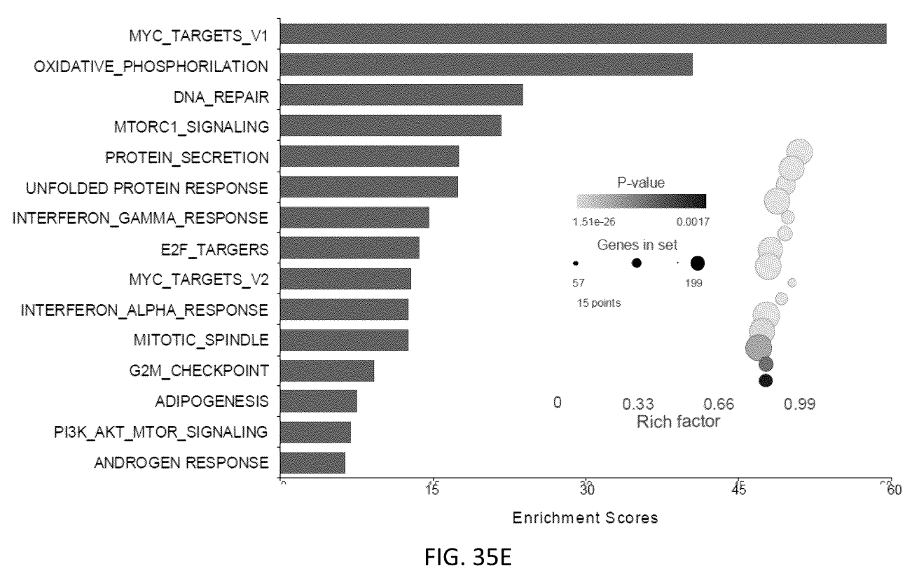

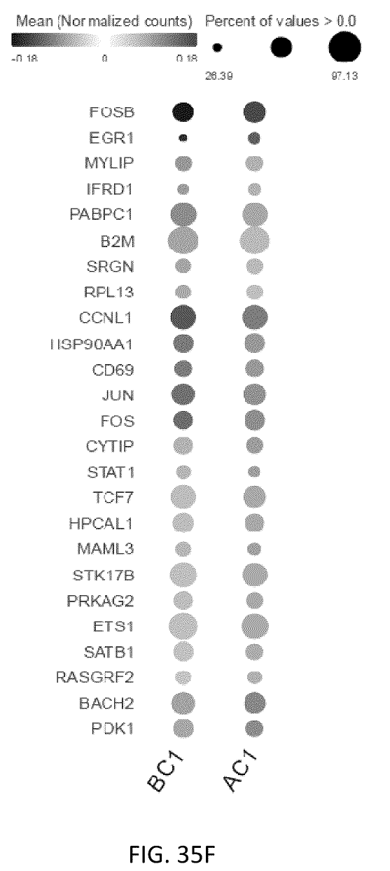

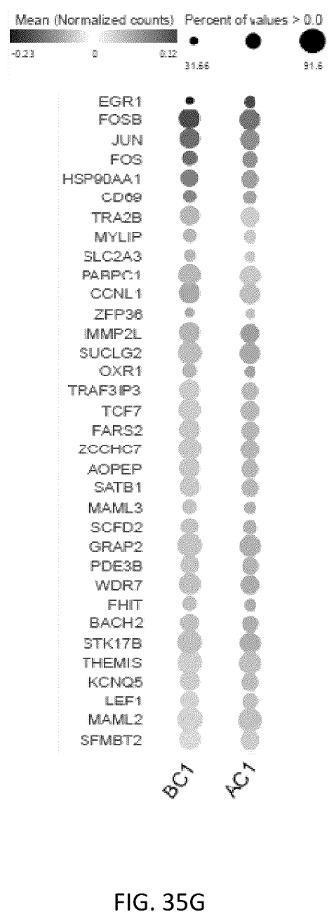

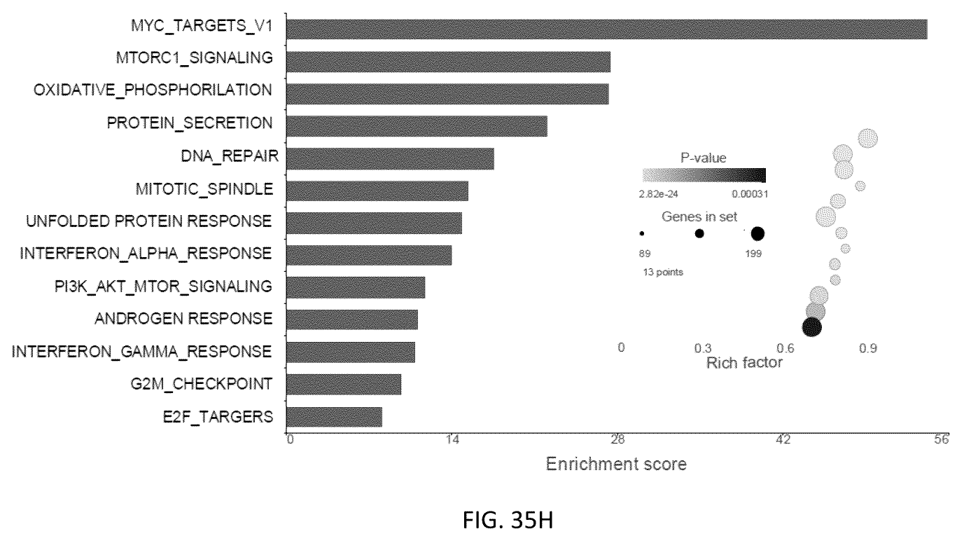

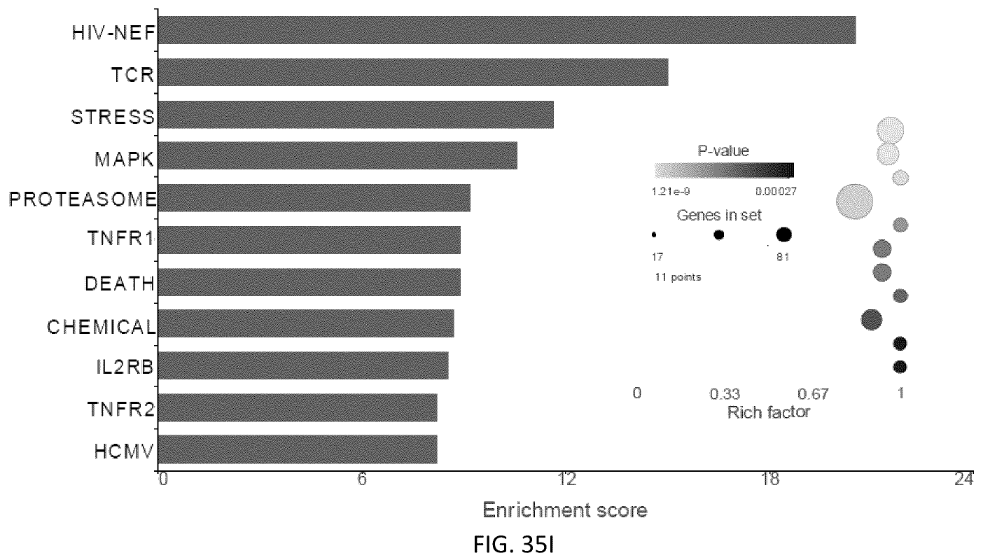

In previous studies, it was shown that 6 hrs after galunisertib treatment in naïve macaques there was an upregulation of the API complex (JUN and FOS) and several genes encoding ribosomal proteins in CD4+ T cells (110). To understand early and later effect of galunisertib in the context of SIV infection, in the current study bulk RNAseq of PBMC isolated 1 hr after the first administration of galunisertib in cycle 1 and at the end of the 1st 2-weeks cycle was performed. 640 genes significantly modulated (FDR q<0.05; log 2FC-2) in PBMC just 1 hr after the first dose of galunisertib. The majority (457 genes) were downregulated (FIG. 32A). Gene set enrichment analysis (GSEA) revealed an upregulation of the oxidative phosphorylation (OXPHOS; ES 0.58 FDR q=0.048)) and the reactive oxygen (ES 0.55 FDR q=0.075) pathways among the Hallmark genet sets (FIG. 32A and FIG. 33). Among the Biocarta sets, there was an enrichment in the electron transport chain (ETC; ES 0.84 FDR q=0.132) and a downregulation of the circadian (ES −0.81 FDR q=0.163) pathways (FIG. 32A and FIG. 33). Finally, among the top modulated genes there were several genes encoding for soluble transporters, while classical activation markers like CD69 and CD38 were downregulated (FIG. 33B). An early engagement of metabolic pathways was confirmed by enrichment analysis of significant DEG with Metascape (116) with an upregulation of adipogenesis, OXPHOS and fatty acid metabolism (FIG. 33C). Of note, 2 weeks after the beginning of galunisertib, metabolic pathways were still among the most enriched upregulated pathways in PBMCs (FIG. 32B and FIG. 34A). Among the top upregulated genes there were CD44, CCR5, several integrins and GRZA and GRZB (FIG. 34B). Finally, we performed bulk RNAseq of rectal biopsy tissue before and after the first cycle with galunisertib (FIG. 32C). There were only 51 differentially 284 expressed genes (DEGs; Adjp<0.05; log 2FC=2). GSEA analysis of all DEGs (Adjp<0.05) revealed the G2_M_DNA replication pathway highly enriched (ES 0.97 FDR q=0.012) within the Hallmark set. Among the most interesting changes, we observed a pronounced downregulation of integrin β7, in contrast to an increase in integrin αE (CD103; FIG. 35C), suggesting that TGF-β-driven increase in «E (117) may be driven by non-canonical TGF-β pathway signaling not blocked by galunisertib.

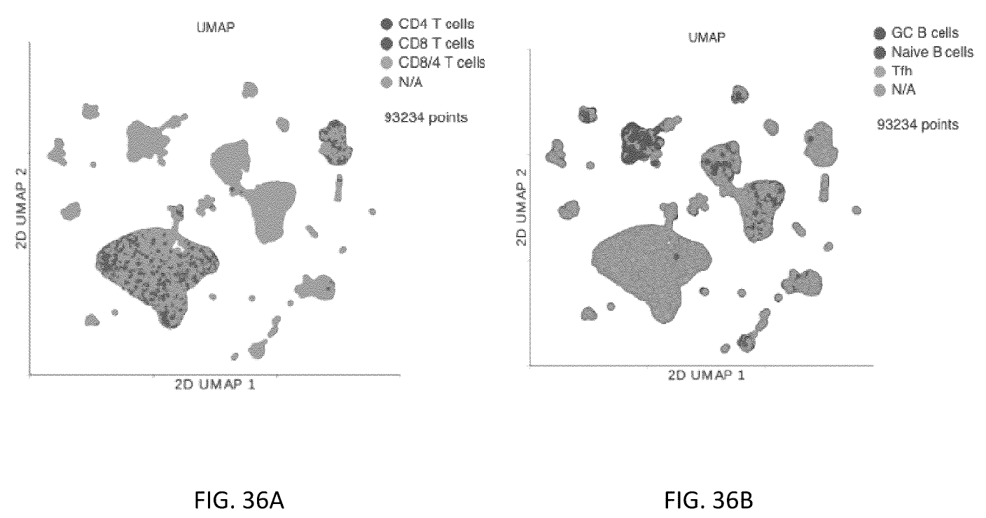

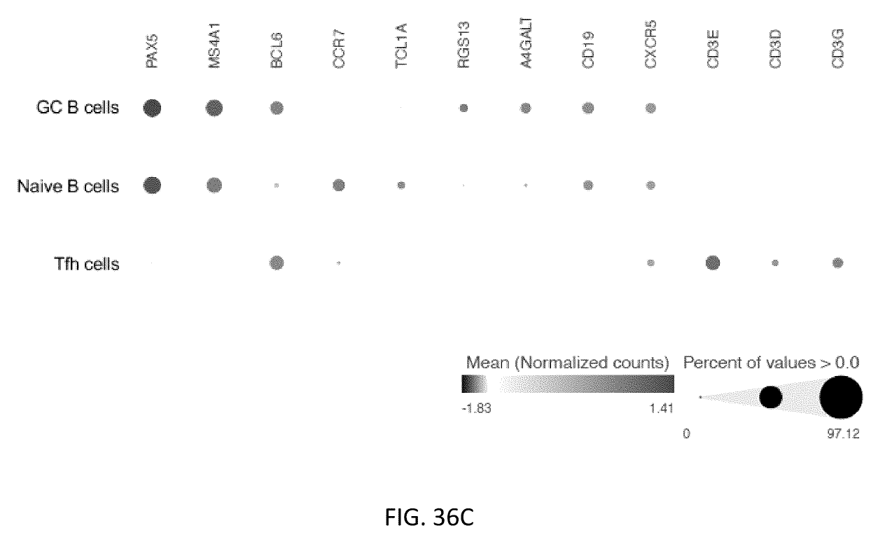

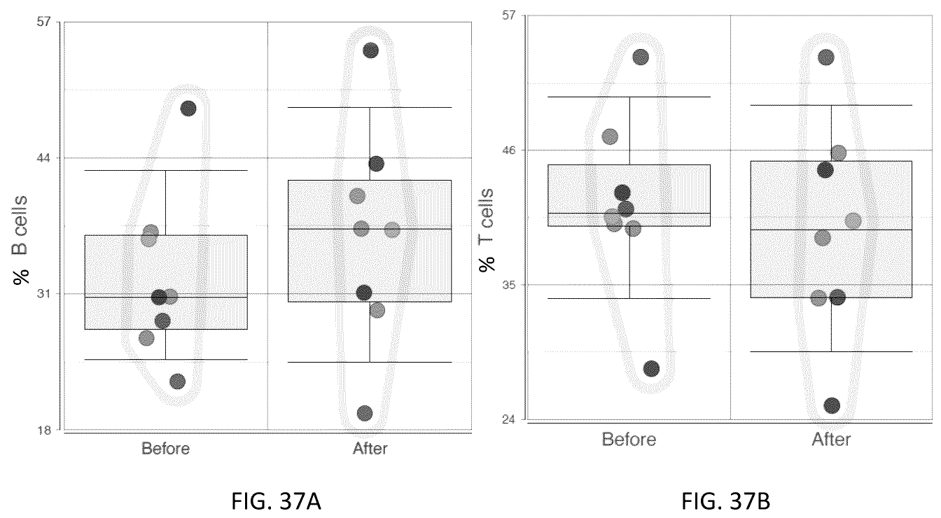

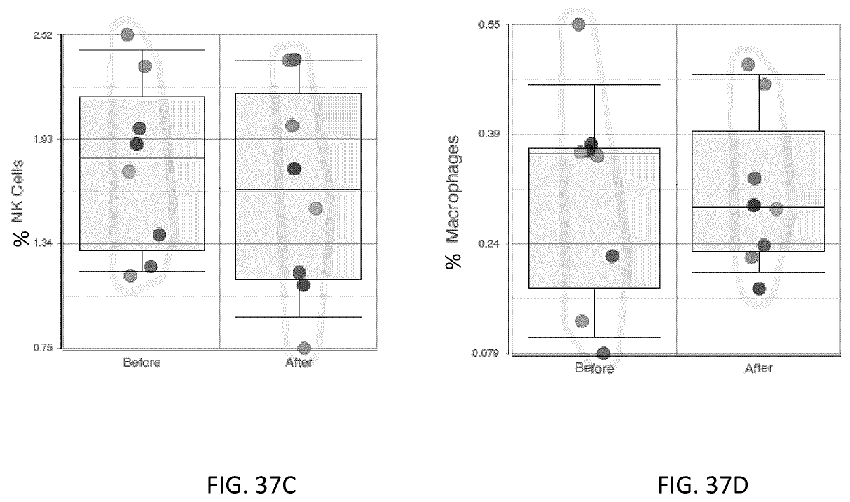

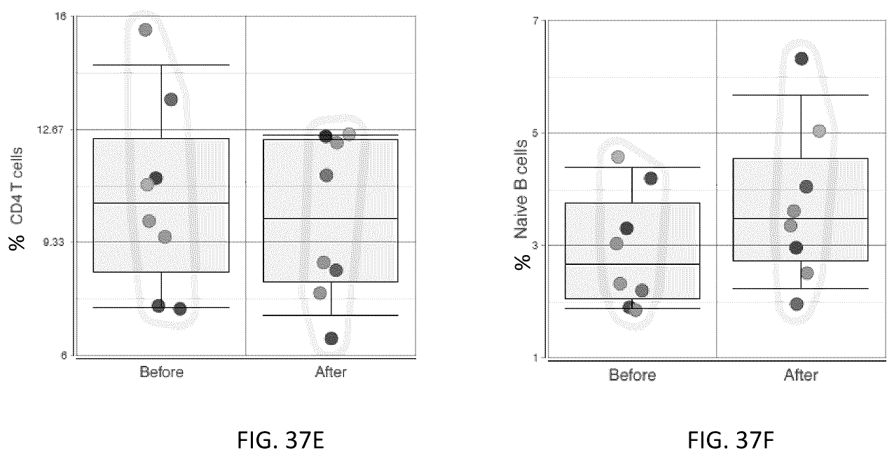

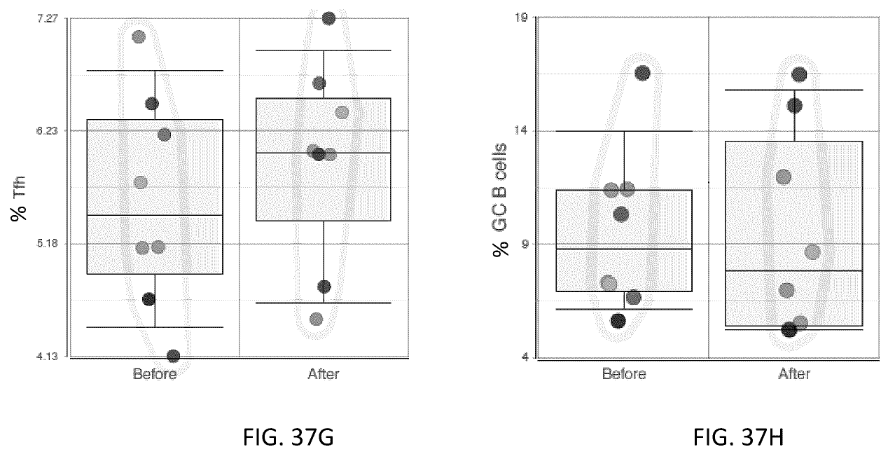

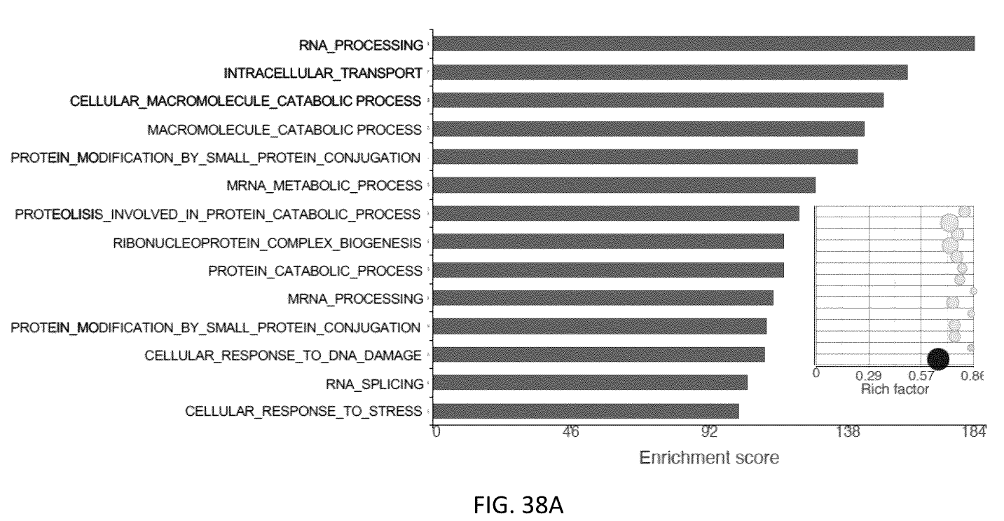

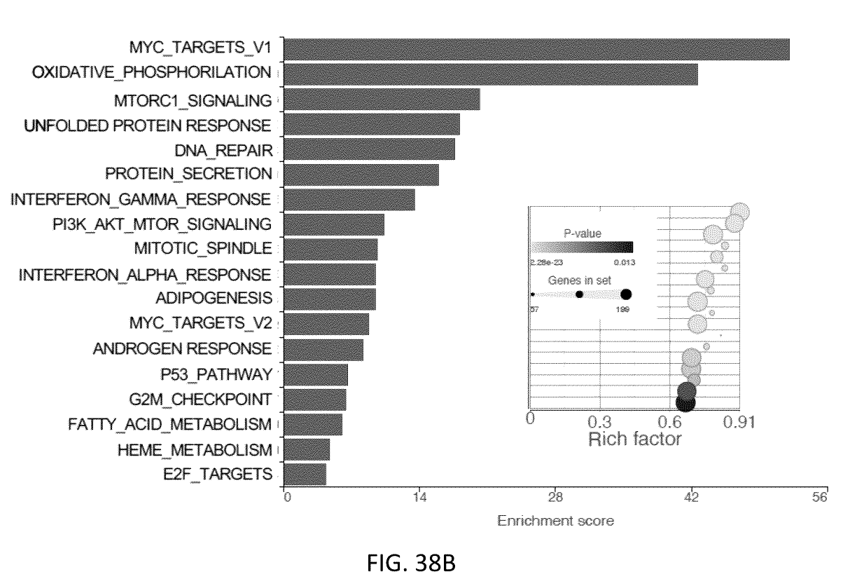

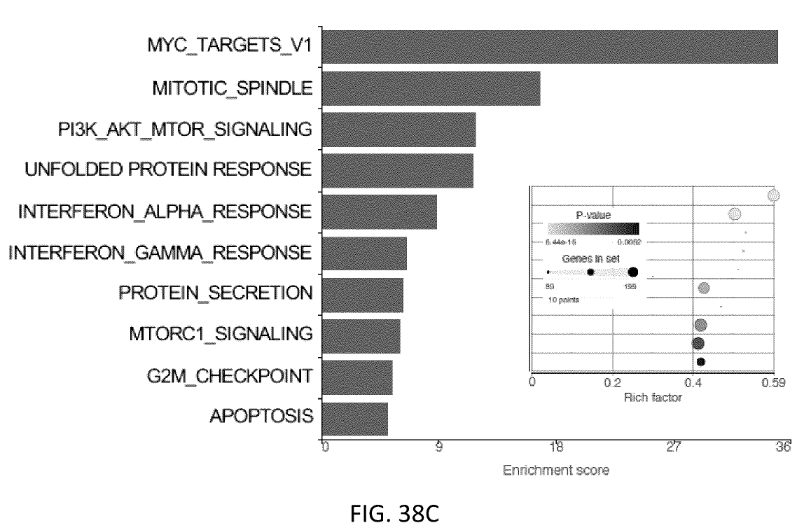

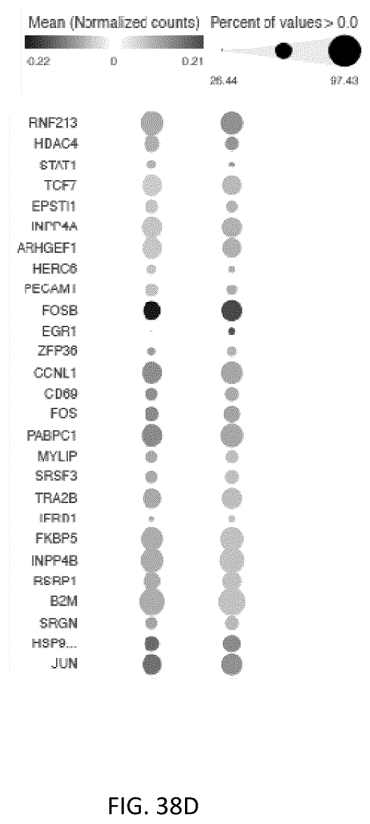

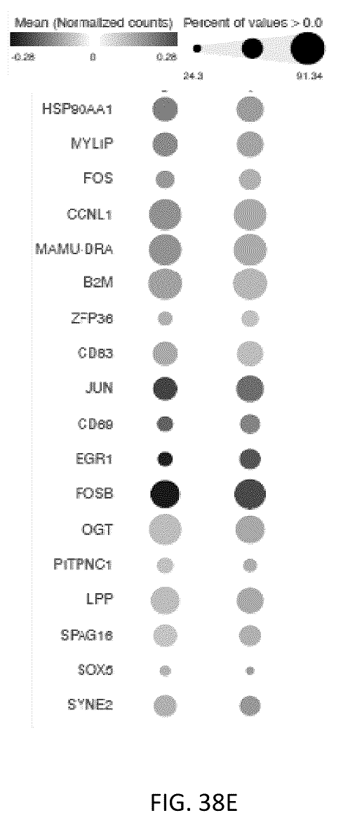

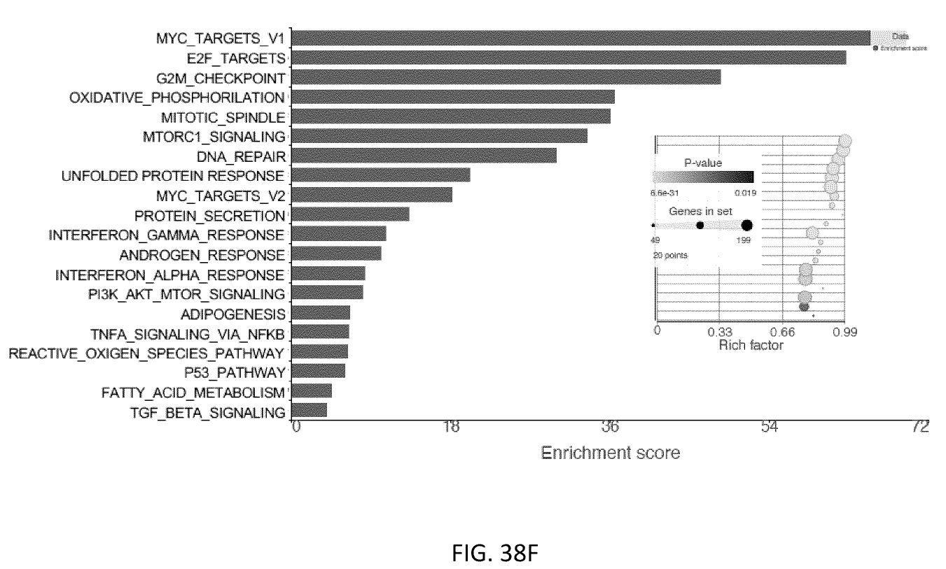

To clarify the impact of galunisertib at the single cell level and in lymphoid tissues, scRNAseq analysis of cells isolated from LNs before (right axillar) and after (right or left inguinal) the first cycle was also performed (FIG. 35). Dimensionality reduction and clustering analysis with PCA and uniform manifold approximation and projection (UMAP)(118) was performed to visualize the data (FIG. 35A). However, cells were classified based only on gene expression (FIGS. 35B and 35C). First, major subsets: T cells (38,705 cells), B cells (31,385 cells), NK cells (1,628 cells), macrophages (285) and cells expressing both CD19 and CD3 (942 T/B cells) were classified (FIG. 35A shows this classification over UMAP). Then only CD4+ and CD8+ T cell subsets (FIG. 35C and FIG. 36A) and B/T subsets including naïve and germinal center (GC) B cells and Tfh cells (FIGS. 36B and 36C) were classified. Cell number for all these subsets did not change with treatment (FIG. 37). However, differential gene expression analysis of T cells revealed an upregulation of members of the API complex, CD69, β2 macroglobulin and RPL13 among the most upregulated genes (FIG. 35D). Moreover, it confirmed a downregulation of TCF1 at the transcriptional level (TCF7 gene; FIG. 35D). Gene enrichment analysis revealed again Myc_targets_V1, OXPHOS and mTORC1 as the most enriched hallmark pathways (FIG. 35E). Of note, among gene ontology cellular processes, RNA processing was the most highly enriched pathway with intracellular transport and catabolic processes following right after (FIG. 38A) confirming an increase in translation and metabolism within these cells.

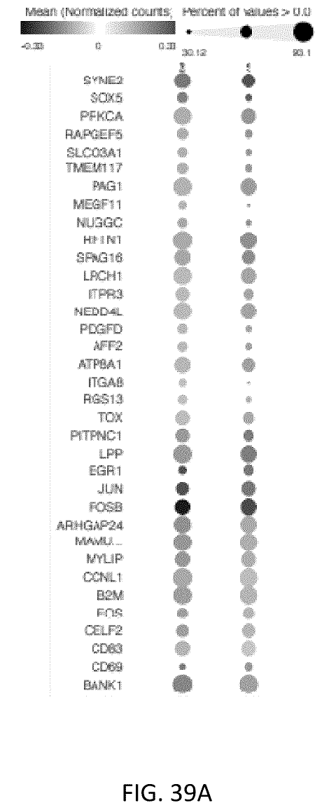

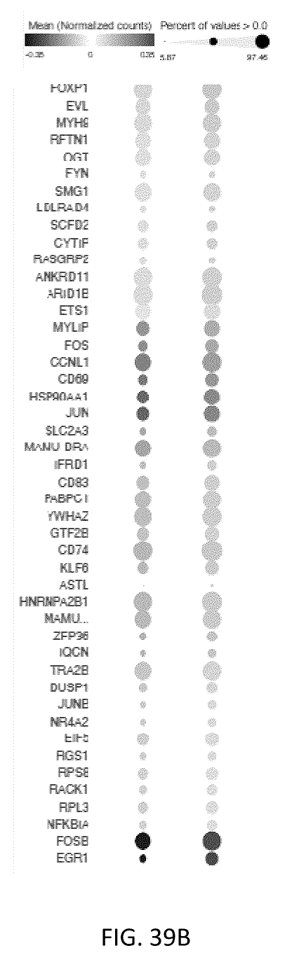

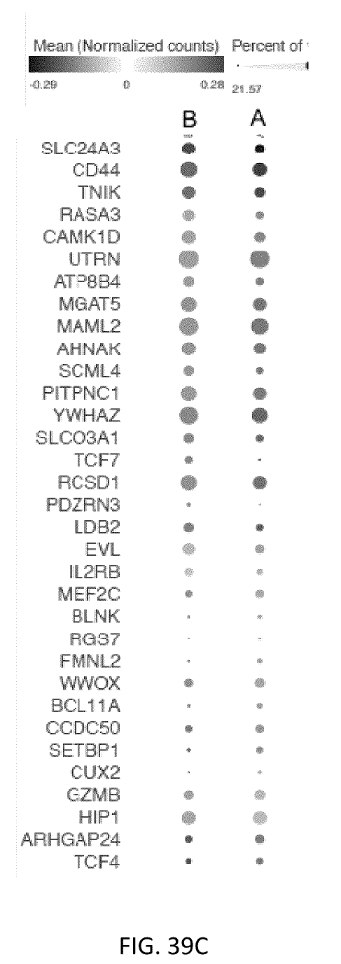

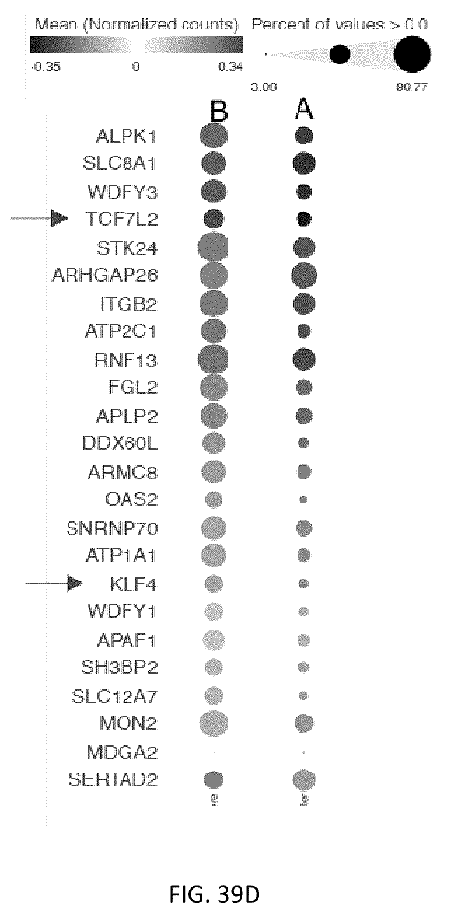

Since there were substantial differences in the impact of galunisertib on CD4+ T cells compared to CD8+ T cells by flow, analysis on these subsets was performed. In CD4+ T cells we found only 25 DEGs with a log 2FC=0.15, while 34 DEGs were in CD8+ T cells with more downregulated genes in the CD8+ T cells compared to the CD4+ T cells. The API complex and TCF7 were again upregulated and downregulated respectively in both CD4+(FIG. 35F) and CD8+ T cells (FIG. 35G). However, STAT1 was more strongly downregulated in CD4+ T cells. Enrichment analysis showed once again upregulation of Myc_targets_V1, OXPHOS and mTORC1 pathways in both CD4+ and CD8+ T cells (FIG. 35H and FIG. 38B). Interestingly, the most enriched set among in Biocarta was the HIV-Nef pathway (FIG. 35J) demonstrating the relevance of these galunisertib-driven changes to HIV cell cycle and transcription (highly enriched KREB pathway as well). The 2nd most enriched Biocarta pathway was TCR signaling, linking galunisertib to cell activation. Next, we analyzed changes in gene expression in Tfh cells. In this subset we obtained a similar number of DEG as in other T cells and myc_targets_V1 was still the most enriched hallmark pathway (FIGS. 38C and 38D). In B cells, the API complex was again prominently upregulated, together with CD83, CD69 and MAMU-DR. Of note, more genes were modulated in B cells (44 genes with a log 2FC=0.15 and 18 with logFC0.2) than in T cells with some differences in enriched pathways (FIG. 38F). Similar genes were modulated in GC B cells and naïve B cells with several more DEGs in naïve B cells than in GC cells (FIGS. 39A and 39B). Finally, 144 genes were modulated in NK cells and 39 genes in macrophages (FIGS. 39C and 39D; log 2FC=0.15). GRZB was prominently upregulated, but CD44 downregulated. In macrophages genes were mostly downregulated including TCF7L2 and KLF4 suggesting an increase in inflammatory phenotype and decrease in M2 polarization (119) (FIG. 39D).

### Example 11—Galunisertib Increases SIV-Specific Responses and Changes Barcode Distribution

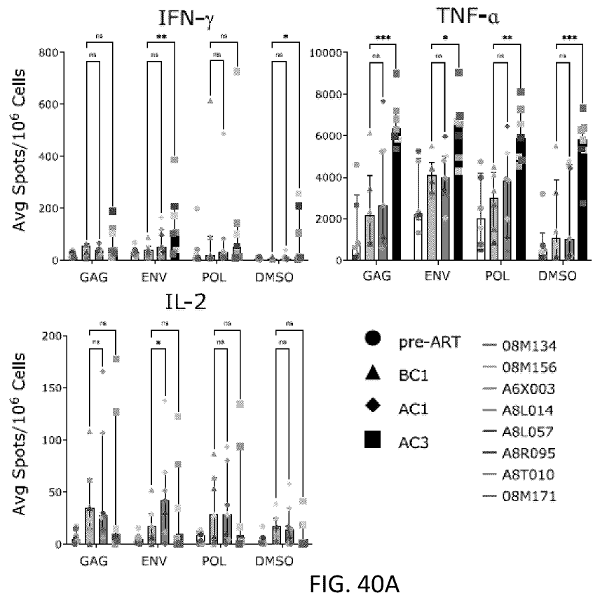

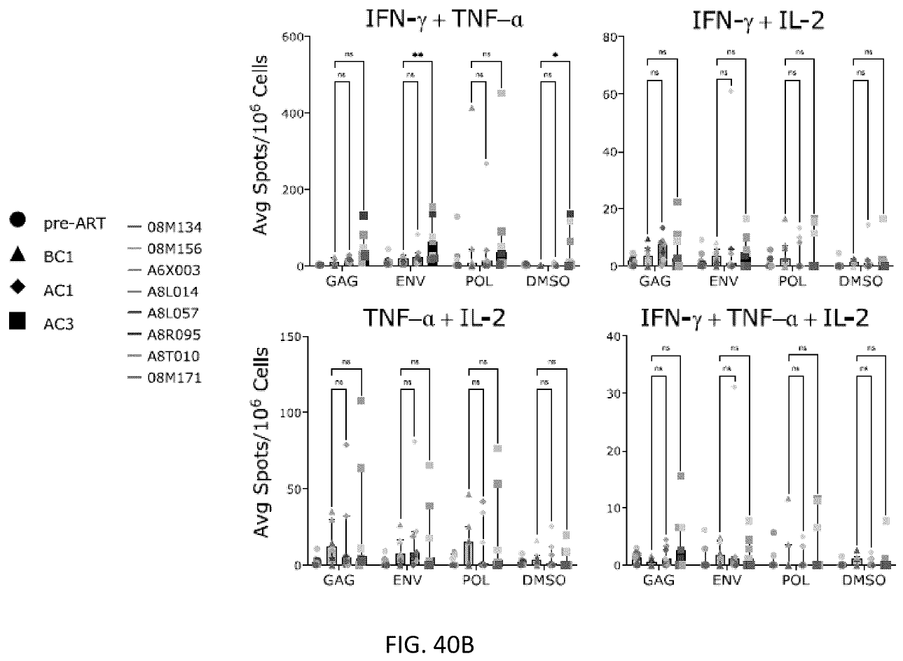

In order to understand how galunisertib affected immune cell function and SIV-specific responses, we stimulated PBMC with 15-mer SIV peptides from SIVmac239 gag, pol and env for 24 hrs on antibody coated Elispot plates. Because of sample availability, before and after the first cycle and after the 3rd galunisertib cycle only was probed. Interestingly, by the end of the 3rd cycle there was a significant increase in IFN-γ secretion both SIV-specific (particularly against env) and non-specific (DMSO control). TNF-α release increased particularly in response to Gag and non-specifically (FIG. 40). However, IL-2 release appeared to increase slightly after the first cycle (non-significant), but remained unchanged with a slight decrease by the end of the 3rd cycle (FIG. 40).

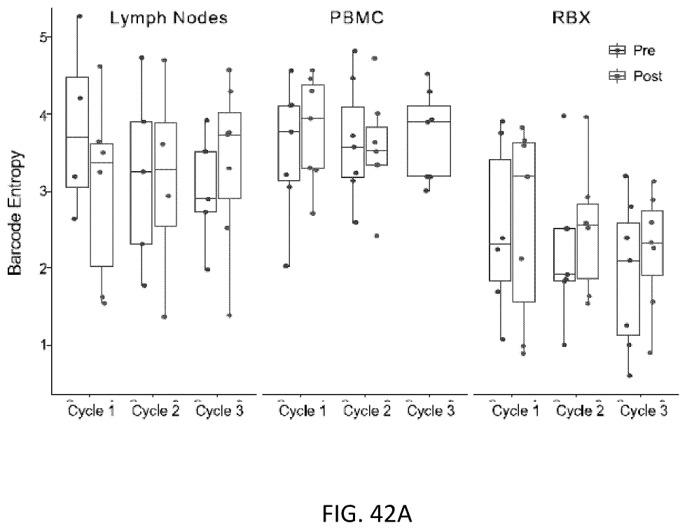

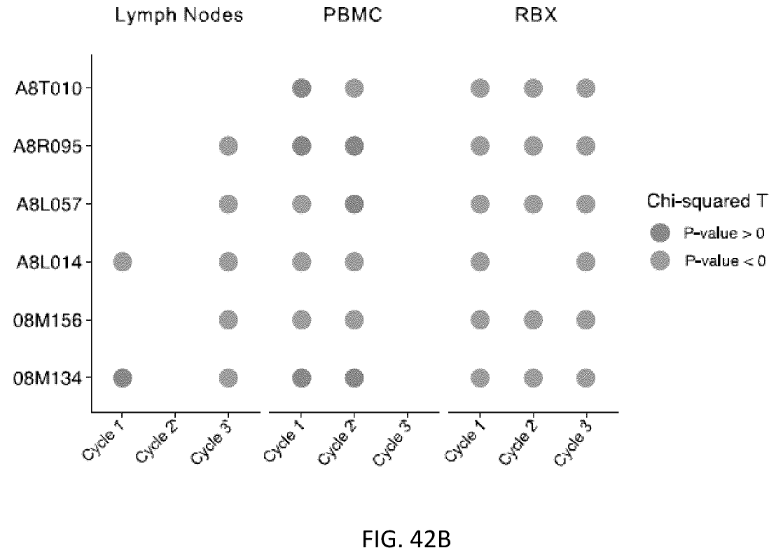

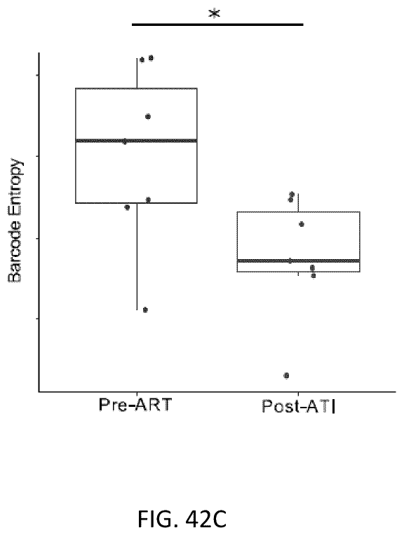

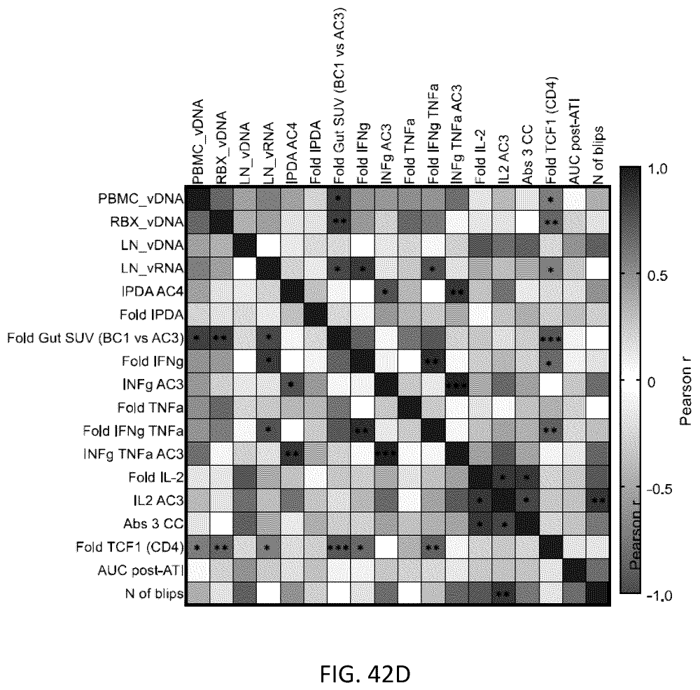

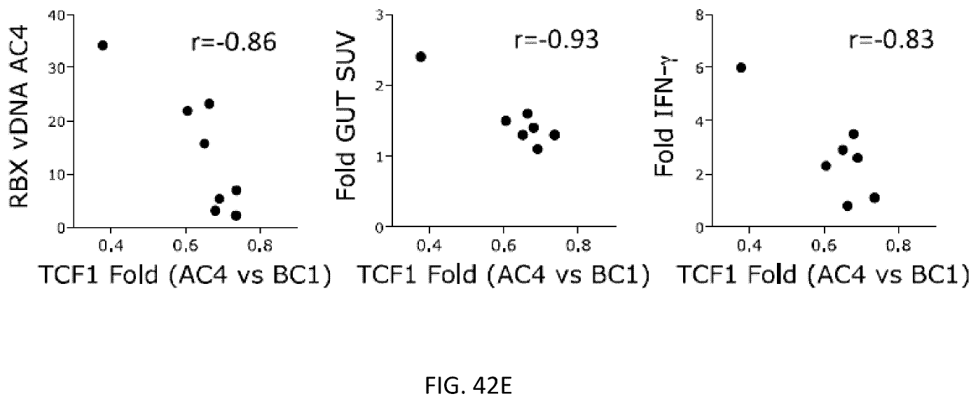

To understand if these changes in immune responses in combination with latency reversal and switch toward an effector phenotype may have impacted viral population dynamics, we analyzed changes in numbers and distribution of the viral barcodes before and after the first 3 treatment cycles. There was no barcode amplification at several time points particularly in the lymph nodes. However, although there were no significant changes in the number or diversity (measured as Shannon Entropy; Sh) of barcodes before and after each galunisertib cycle (FIG. 41A and FIG. 42A), significant changes were found in barcode frequency distribution in most tissues and cycles after galunisertib treatment (FIG. 41B-41J and FIG. 42B). Specifically, the relative proportion of each barcode changed in all monkeys in all cycles in the rectal biopsies (probably a consequence of different sampling area), but also for all LN analyzed (except for 08M134 in cycle 1). Of note, the same LN were sampled at the beginning and after cycle 3 (Table 5). Hence, sampling location does not explain the changes barcode distribution. Changes in the proportion of barcodes were also detected in at least half of the PBMCs after cycle 1 and 2 (cycle 3 not analyzed). Finally, barcode diversity decreased in plasma post-ATI compared to the time point right before ART (FIG. 42C) and barcode distribution significantly changed post-ATI compared to pre-ART in 2 of the 7 macaques that rebounded (FIG. 41J).

TABLE 5
List of LN sampled (FNA biopsy or surgical biopsy)

| | Peak | pre- | Cycle 1 | | Cycle 2 | | Cycle 3 | | Cycle 4 |
|---|---|---|---|---|---|---|---|---|---|
| | VL | ART | BC1 | AC1 | BC2 | AC2 | BC3 | AC3 | AC4 |
| | W 2 | W 6 | W 35 | W 37 | W 39 | W 41 | W 43 | W 45 | W 49 |
| 08M134 | RI, RA | RA | RA | RI | LA | LA | RA | RA | RA/LA |
| 08M156 | RI, RA | LI | RA | LI | LA | LA | RA | RA | RA/LA |
| A6X003 | RI | RA | RA | RI | LA | LA | RA | LA | RA/LA |
| A8L057 | RA, LA | RA | RA | RI | LA | LA | LA | LA | RA/LA |
| A8L014 | LI, RI | RI | RA | RI | LA | LA | LA | LA | RA/LA |
| A8R095 | LI, RI | RI | RA | LI | LA | LA | RA | RA | RA/LA |
| A8T010 | RA, LA | RA | RA | LA | LA | RA | RA | LA | RA/LA |
| O8M171 | | | LA | LA | RA/RI | LA | LA | RI | RA/LA |

RI = Right inguinal
LI = Left inguinal
LA = Left Axillary
RA = Right Axillary

### Example 12—TCF1 Downregulation in CD4+ T Cells Correlates with Virological and Immunological Endpoints

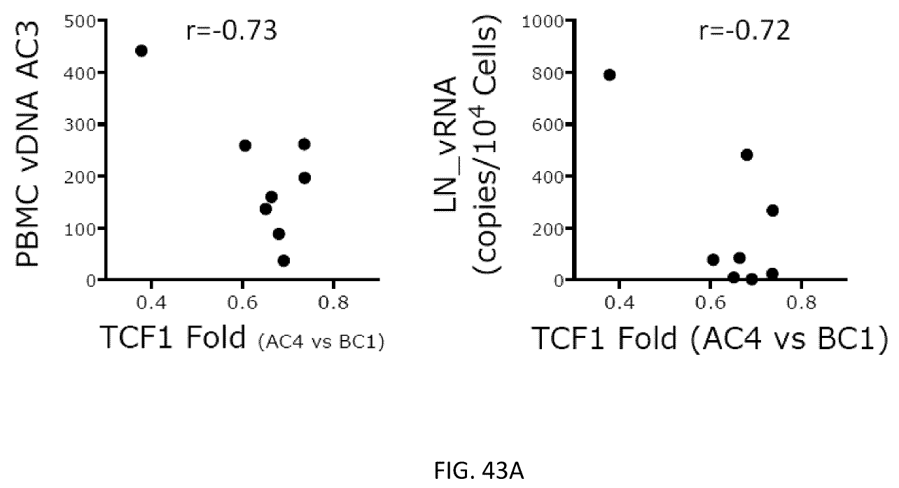

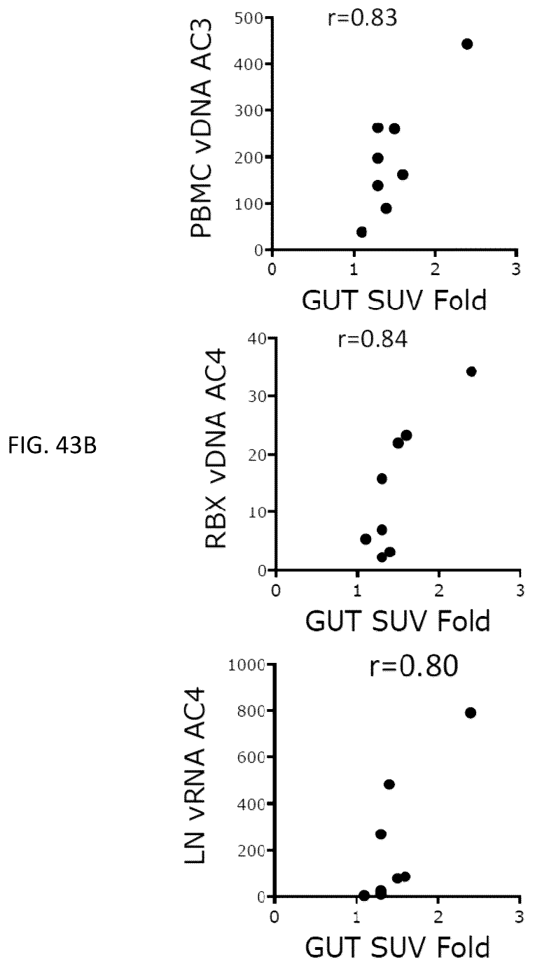

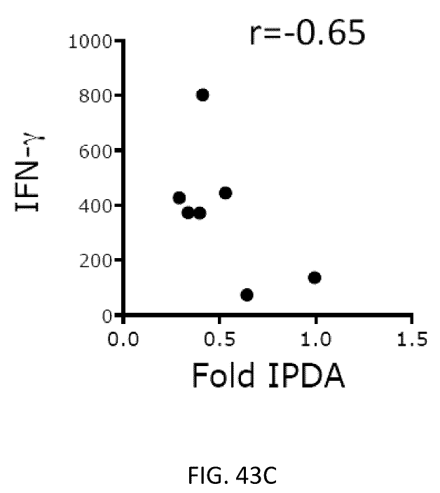

To explore a possible association between the various virological and immunological parameters and their changes, we built a correlation matrix with a curated set of variables of interest or their fold changes. This analysis revealed an association between the decrease in TCF1 expression in the CD4+ T cells and several virological and immunological variables (FIG. 42E). Specifically, both the levels of CA-vDNA in the colorectal tissue and the fold increase in gut-SUV strongly inversely correlated with fold changes in TCF1. Since TCF1 decreased, a larger decrease in TCF1 was directly proportional to residual vDNA in the gut at the end of cycle 4 and to the increase in PET signal in the gut (FIGS. 42D and 42E). A weaker, but still significant association was also present with CA-vDNA in PBMC at the end of cycle 3 and with the levels of vRNA in the lymph nodes at the end of the 4th cycle (FIG. 42D and FIG. 43A). Finally, the decrease in TCF1 correlated with the increase in IFN-γ and poly-functional IFN-γ/TNF-α releasing cells (cumulative increase of SIV-specific responses to gag, pol and env; FIGS. 42D and 42E). Of note, the increase in PET signal in the gut also correlated with the levels of vDNA in PBMC at the end of cycle 3, vDNA in gut biopsies and residual CA-vRNA in LN at the end of cycle 4 (FIG. 43B). Interestingly, the levels of residual CA-vRNA in LN also correlated with the increase in IFN-γ and poly-functional IFN-γ/TNF-α releasing cells (Pearson r-0.85 and 0.76, respectively). Finally, the residual intact pro-virus (IPDA) directly correlated with the levels of IFN-γ and IFN-γ/TNF-α produced in response to SIV peptides at the end of cycle 3 (FIG. 42D; r=0.76 and 0.88, respectively). In contrast, the change in intact provirus tended to correlate inversely with the levels of IFN-γ, so that a larger decrease directly correlated with more IFN-γ responses. However, this did not reach significance (p=0.117, FIG. 43C).

### Discussion

HIV-1 latency in T cells is maintained through diverse mechanisms that include blocks in transcriptional elongation, completion, and splicing(2). A common characteristic shared by HIV-latently infected cells of both T and myeloid cell lineages is their “resting” phenotype(13, 120-122) In these cells, an inability to transcribe proviral DNA is linked to a generalized decrease in transcriptional activity which, in turn, is linked to their metabolic status(96). Cellular metabolism is in turn influenced by tissue location and environmental cues(123).

TGF-β is released at high level in PLWH and its levels remain high during ART(124). The immunosuppressive activity of TGF-β is well-known. However, the effect of TGF-β signaling in immune cells is highly context dependent(125). Hence, TGF-β plays different roles according to a cell differentiation and activation status(125). In CD8+ T cells and NK cells, TGF-β was shown to decrease mTOR activity and preserve cellular metabolism (high mitochondrial activity and spare respiratory capacity, but reduced mTOR activity) preventing metabolic exhaustion(98, 99, 126, 127). This effect was linked to survival of antigen-specific CD8+ T cells, preservation of their stemness and it was linked to higher expression of the TCF1 factor(98).

In contrast, in CD4+ T cells TGF-β is known to decrease TCR activation(71, 127), restrict proliferation and inhibit cytotoxicity (including granzyme and perforin release) at different stage of infection in vivo(102, 129). However, the role of TGF-β in the formation and preservation of CD4+ T cell memory is still unclear(125). Moreover, the link between TGF-β signaling and TCF1 expression in CD4+ T cells is unexplored.

Here, a clinical stage small drug, galunisertib, developed by Eli Lilly and used in several phase 1 and 1/2 clinical studies against solid cancer(43, 44, 130) was used to investigate the impact of TGF-β blockade on SIV latency, SIV reservoir and immune responses. Of note, Eli Lilly did not terminate galunisertib development program because of toxicity(131, 132). Indeed, in our studies in macaques, we observed no adverse events nor changes in chemical or hematological variables. Moreover, there were no detectable changes in the levels of the inflammatory factors that we probed in plasma during the treatment. This suggests that this therapeutic approach may be safe in people living with HIV (PLWH).

The first important finding of this study was the confirmation of our previous report of the latency reversal properties of TGF-β blockade in vivo(107). Indeed, we found increase in pVL in 7 out of the 8 macaques upon initiation of galunisertib therapy. Although not all macaques were fully suppressed before treatment, substantial increases in pVL (>102 copies/ml) were noted also in fully suppressed macaques (08M171, A8R095 and A8L057). Moreover, extensive viral reactivation was documented in tissues by immunoPET/CT. The increase detected post galunisertib in gut SUV correlated with CA-vRNA as in our previous studies(107) and, interestingly, it was associated with a decrease in TCF1. Galunisertib led to increased probe uptake in the gut and lymphoid tissues, as well as a generalized increase in whole-body uptake. Yet, there was no change in the PET background signal in kidneys and spleen. This, in conjunction with our previous studies demonstrating the specificity of the PET signal for areas of enhanced SIV replication in gut and lymph nodes(62, 63, 108), demonstrates that the galunisertib-driven increases in SUV were mostly specific, and highlighted areas of SIV latency reversal at least in gut and lymphoid tissues. The extent to which the increased signal in the spine and bones recapitulates an increase in SIV replication at these sites remains to be determined.

Importantly, we observed a decrease in CA-vDNA in all the tissues that cannot be attributed to ART alone. Indeed, although we did not have a concurrent control group, this decrease was not present in similar studies conducted in SIVmac239M2 infected macaques on the same ART regimen, but not treated with galunisertib. Considering studies by other groups with different models (SIVmac), they report 2nd phase decay of SIV intact provirus (weeks ˜32 to ˜100 pi, FIG. 2B in(133)) with a t1/2 of >8 months(133). In contrast, in our study, the intact provirus decreased by 3 folds (median) in a little over 3 months (from week 35 pi, BC1 to week 49 pi, AC4). Interestingly, this decrease in intact pro-virus trended toward a direct correlation with IFN-ã responses. However, IFN-ã levels also inversely correlated with the absolute value of residual intact provirus, suggesting that IFN-ã responses were driven by residual viral reservoir while, at the same time, were involved in clearing intact virus. Indeed, the increase in IFN-ã and TNF-á also correlated with residual CA-vRNA in the LN at the end of the treatment. The latter, in turn, was directly proportional to the increase in gut SUV. This is in line with increased latency reversal explaining residual viral RNA in lymphoid tissues.

Finally, one of our most intriguing results was the profound downregulation of TCF1 in CD4+ T cells at both the transcriptional and protein levels. Although TCF1 is conventionally viewed as an effector of the canonical Wnt pathway(134) and recently reached notoriety for its role in maintaining stemness of antigen-specific memory CD8+ T cells(99,135), TCF1 has a plethora of functions in T cell development and differentiation largely independent of Wnt signaling(95). In CD4+ T cells, TCF1 has been implicated in orchestrating all the major Th subsets, including Th1, Th2, Th17 and Tfh(136). TCF1 is known to control the bifurcation between Th1 and Tfh in favor of Tfh cells(136), while it negatively regulates Treg development(95). This is in line with our findings of increased Treg and decreased Tfh, in the midst of a profound downregulation of TCF1. Importantly, TCF1 is downregulated with increased cellular differentiation and progression toward effector functions in T cells. T cell activation leads to reduced levels of TCF1 and higher levels of TCF1 are present in T cells with higher stemness and low anabolic metabolism(134). These findings suggest that TCF1 has a critical role in maintaining quiescence in immune cells likely in concert with TGF-β(91, 129). Our data reveal that this link may be even more prominent and important in CD4+ T cells than in CD8+ T cells. Importantly, the decrease in TCF1 was accompanied with enhancement in other effector markers such as CD95, CD16 and GRZB (at the transcriptional level) and an increase in the transcription of API complex. However, there was no clear upregulation of other classical markers of immune activation such as CD69 and no increase in T cell proliferation (Ki67 expression). Hence, galunisertib treatment does not appear to lead to classical T cell activation. Instead, in vivo TGF-β blockade seems to primarily change the metabolic state of T cells (and likely other immune cells) increasing OXPHOS and mitochondrial function. Of note, although glycolysis is essential during cell activation, mitochondrial pathways are engaged and remodeled early after activation and OXPHOS upregulation has a pivotal role in the earliest stages of cell activation(91). Hence, we propose a model in which TGF-β inhibition forces cells (particularly CD4+ T cells) out of quiescence to a transitional state where they reinitiate their transcriptional program and are metabolically ready to be activated and effector. In absence of direct TCR engagement or other activation stimuli, they do not undergo full/classical activation and proliferation, yet they can reinitiate viral transcription and respond to SIV antigenic stimulation. Indeed, functionally, the PBMC after galunisertib treatment were demonstrated to secrete higher levels of IFN-ã and TNF-á. Interestingly, there was no increase in IL-2 secretion. The link between TGF-β and IL-2(137) and the critical role of IL-2 in T cell proliferation again suggest that galunisertib enhances an effector phenotype uncoupled from cellular proliferation. scRNAseq analysis demonstrated a trend toward an effector phenotype also in other immune cells, such as B cells, NKs and macrophages. Future studies will need to uncover in depth the effect of TGF-β blockade on these other immune subsets.

### Materials and Methods

Study design and Ethics Statement

A total of 8 adult female Indian origin Rhesus macaques (Macaca mulatta; Mamu A*01, B*08 and B*17 negative) were used for the study described in this example (Table 3). All the macaques were selected form the colonies bred and raised at the New Iberia Research Center (NIRC), University of Louisiana at Lafayette. Treatments were in accordance with NIRC standards which comply with the Guide for the Use and Care of Laboratory Animals. Any abnormal observations and/or signs of illness or distress were reported per NIRC SOPs. Appropriate procedures were performed to ensure that potential distress, pain, discomfort were limited to that unavoidable in the conduct of the research plan. Ketamine (10 mg/kg) and/or telazol (4 mg/kg) sedation were used for sample administration, collection of samples and conducting image collections. Analgesics were used when determined appropriate by veterinary medical staff. Monkeys were fed Teklad monkey chow supplemented with fresh fruit or vegetables and water ad libitum.

Rhesus macaques (n=8 main study+4 separate non-concomitant study) were infected with 300 TCID50 of the barcoded SIVmac239M2 stock intravenously and ART (Tenofovir [PMPA], Emtricitabine [FTC] and Dolutegravir [DTG]) was initiated on week 6 pi. Galunisertib treatment was initiated on week 35 p.i. Powder (MedChemExpress—MCE, NJ, USA) was dissolved in water and given orally in a treat twice daily at 20 mg/kg. 4 cycles of 2 weeks daily treatment with 2 weeks wash out period were performed. Macaques 08M156 and A6X003 were given the rhesus recombinant antibody (rhesus/human chimeric) anti-PD1 antibody [NIVOR4LALA; comprising silenced rhesus IgG4k 510 constant regions and variable regions from anti-human PD-1, nivolumab; non-human primates reagents resource, NHPRR. Blood viral load was monitored biweekly before and during ART and every 3-4 days during Galunisertib treatment. Colorectal biopsies and LN FNA were collected before and after galunisertib treatment. ART was terminated 3 weeks after the last galunisertib dose and euthanasia and necropsy to harvest tissues were performed at week 58 post infection, tissues samples were flash frozen, fixed in OCT or Z-fix.

### Plasma and Tissue SIV Viral Loads

Blood was collected in EDTA tubes and plasma was separated by density gradient centrifugation and used for the determination of plasma VL by SIVgag qRT-PCR at NIRC or at Leidos (Quantitative Molecular Diagnostics Core, AIDS and Cancer Virus Program Frederick National Laboratory). Tissue VL from snap frozen PBMC pellets, colorectal biopsies and LN FNA were performed as described in (77). SIV-TILDA was performed on freshly stored PBMC before cycle 1 and at the end of cycle 4 of galunisertib by Accelevir, Baltimore, MD.

### ImmunoPET/CT

ImmunoPET/CT for mapping SIV signals in total body scans were conducted in part as reported previously (110). The probe consisted of primatized p7D3 anti-env F(ab)′2 coupled with the chelator DOTA and labeled with Cu64 just prior to administration to the animals. For probe administration, the animals were sedated, and a venous catheter was placed into an arm of leg vein to minimize bleeding of the probe into the tissue surrounding the site of injection. The probe for each animal consisted of ˜1 mg of the p7D3 F(ab)′2 labeled with 2-3 mCi of ⁶⁴Cu. After injection, the animals were allowed to recuperate in their cage until the next day. At 24 hours, the animals were again anesthetized with Telazol and the macaque's body was immobilized in dorsal recumbency on the scanner table. Scans were conducted 535 in a Phillips Gemini TF64 scanner. The final CT image was compiled from 200 to 300 slices, depending on macaque size. PET Image analysis was performed using the MIM software. PET/CT fusions were generated scaled according to calculated Standardized Uptake Values (SUV). The SUV scale for the PET scans was selected based on the overall signal intensity of the PET scans (whole body), and the CT scale was selected for optimal visibility of the tissues. All images and maximum image projections were set to the same scale for visual comparisons.

### ImmunoPET/CT Analysis

Multimodal registration and ROI segmentation were executed using the MIM software (MIM Software, Inc., Cleveland, OH) for PET and CT images, encompassing 250 to 300 fractions, depending on the macaque's body size. PET and CT images were fused to quantify changes in SUV (Standardized Uptake Value) within Regions of Interest (ROI), with PET SUV scale determined by overall signal intensity and CT scale optimized for tissue visibility. Segmentations were performed for various anatomical regions: Whole Body, Liver, Lymph nodes, Gut, Kidneys, Spleen, and Vertebrae. The external body was outlined for the Whole-Body contour, while the GRADIENT method PET-Edge and interactive contouring tools were employed for other ROIs. Liver segmentation initially utilized PET-Edge in PET coronal-sectional view, followed by interactive delineation for the structure and the corresponding signals. Kidneys, Spleen, and Vertebrae volume were determined based on CT scans in the cross-sectional view, incorporating PET signals into contours. Notably, the analysis for vertebrae was confined to the range from the thoracic vertebrae to the lumbar vertebrae. To quantify the signal in the axillary lymph nodes, the data-point in each macaque featuring the strongest PET signal was chosen as a basis for an inhouse Multi-Time-Point Contour Transfer Workflow, followed by interactive contouring of the signal to all time points. The workflow integrated CT scans to establish and map specific ROIs in subsequent scans, thereby accounting for any changes in the animal's orientation at other time points through manual adjustments. To quantify the signal in the gut tissue, the body segment below the stomach to above the cervix was initially isolated. The Gut's SUV was then calculated by extracting the spleen, both kidneys, liver, and bones within the designated body region using Boolean operations.

### Clinical Pathology at NIRC

1 mL of blood was collected in EDTA tubes and tested for a standard hematology panel (complete blood count—CBC) using a BECKMAN COULTER DxH600 hematology analyzer. An additional 1 mL of blood was collected into a SST tube, centrifuged to isolate serum, and analyzed for a comprehensive serum chemistry panel using a Siemens's Dimension Clinical Chemistry Analyzer.

### Cell Isolation, Flow Cytometry Staining, Classical and High-Dimensional Analysis

Colorectal biopsy tissues were isolated by enzymatic digestion while LN biopsies were passed through a 70 μm cell strainer as described in (110). Isolated cells were phenotyped with panels listed in Table 4. FlowJo vs 10.8 was use for both classical and high-dimensional analysis. PeacoQC, FlowtSNE and FlowSOM plug-ins were used with default settings. SwiftReg was used for normalization (113).

Cell Isolation from Tissues, Flow Cytometry Staining and Analysis

Rectal biopsies were incubated in HBSS containing 2 mg/mL Collagenase IV (Worthington Biochemical) and 1 mg/ml of Human Serum Albumin (Sigma-Aldrich), shaking at 37° C. for 30 minutes. The resulting cell suspension was passed through a 40 μm cell strainer and washed with PBS. Cells from lymph nodes were isolated by passing the collected tissues through a 70 μm cell strainer. Isolated cells were frozen in liquid nitrogen for phenotyping and stimulation experiments at a later time.

Peripheral blood mononuclear cells (PBMCs) collected from 8 macaques at 7 time points spanning Cycle 1 to Cycle 4 (refer to Table 5) were employed for high-parameter flow cytometry staining. Cells from before and after cycle 1 and at the end of cycle 3 were used for Tfh/Treg assay. In both cases, cell fixation and permeabilization for intracellular staining were carried out using the FoxP3 Staining Buffer set (Thermo Fisher Scientific, Carlsbad, CA). The high-dimensional panel consisted of antibodies outlined in Table 4, catering to requirements for surface and intracellular staining, as well as viability assessment. Each antibody was individually titrated to ensure optimal staining conditions. Data acquisition was performed using the FACSymphony A5.2 Spectral Analyzer. Following data collection, spectral compensation was implemented utilizing FlowJo™ v10.8 Software (BD Life Sciences). 60,000 live singlet lymphocytes from each of samples was downsampled to prevent disproportionate representation by samples with higher cell counts, ensuring balanced subsequent analysis. To mitigate batch effects, we utilized the swiftReg Neighbor-Dependent Cluster Registration (NDCR) method available within the SWIFT package(112). This method effectively addresses batch variations while preserving the inherent biological diversity. The NDCR method is particularly adept at registering samples without requiring an internal reference, achieving correction through a calculated value shared among clusters with comparable channel values. Furthermore, we implemented the Peak Extraction and Cleaning Oriented Quality Control (PeacoQC) procedure (138) in FlowJo™ v10.8 to eliminate low-quality events arising from potential issues like clogs, speed fluctuations, and slow sample uptake. FlowSOM FlowJo plugin was employed for automated cell population identification, preceded by the utilization of Phenograph to obtain optimal cluster numbers for dimensional reduction, for the merged data of All Time points and BC1 vs AC4 merged data respectively. The Supplementary FIG. 29A heatmap illustrates the median fluorescence intensity (MFI) values for all markers across all clusters. A tSNE plot, generated in FlowJo™ using the markers employed in the FlowSOM analysis, is presented, with coloring based on the FlowSOM clustering or two specific time points, BC1 vs AC4 in FIG. 4G.

Statistical analysis of Spectral flow cytometry data was executed using Python. To discern disparities in the 36 clusters resulting from FlowSOM between BC1 and AC4, a Wilcoxon test with FDR correction was employed. To evaluate variations within the 38 clusters arising from FlowSOM analysis across multiple time points, a repeated measures ANOVA was conducted with Holm-Sidak correction for multiple comparisons.

### Single-Cell RNAseq Analysis

Cells were isolated from LN biopsies as described above and fixed with the Parse fixation kit, barcoded and sequenced at the NUSeq Core. Libraries were validated by capillary electrophoresis on a Fragment Analyzer (Agilent), and sequenced using 1 S4 flow cell of the Illumina NovaSeq 6000 yielding >20,000 reads/cell. Raw sequencing data were demultiplexed, aligned to Mmul10 and QC with the Parse pipeline. The Partek software was used for the remaining QC. We filtered out cells with less than 400 and more than 8000 features, excluded features with 0 reads in >99.99% of cells, scran deconvolution was used for normalization and cell classification was based on gene expression. Since mitochondrial counts were already below 2% after these filters, we did not filter further. Scran deconvolution was used for normalization and principal component analysis (PCA) and Uniform Manifold Approximation and Projection (UMAP) for data dimensionality reductions and visualization. Cells were classified as T cells if expressed any CD3 (CD3D, CD3E, or CD3G), but no CD19, PAX5 or MS4A1; B cells if they expressed CD19 or MS4A1 or CD79A or PAX5 but no CD3; NK cells if they expressed NCAMI or NKG7 or KLRB1 but no CD3, CD14, MS4A1, CD19 or CD68. Finally, macrophages were CD68+, but CD19− CD79A-LAMP3− and CD3−. In a separate classification, CD4 T cells were classified as expressing CD3 and CD4 or CD8 (CD8A or CD8B), but no CD19, MS4A1 or CD68. Finally, a third classification classified GC B cells as A4GALT (CD77)+ or BCL6+ or RGS13+ and CD19+ or PAX5+ or MS4A1+, but CD3−; naïve B cells as TCLIA+ or CCR7+ and CD19+ or PAX5+ or MS4A1+ and CD3−; Tfh cells as expressing CD3 and BCL6+ or CXCR5+ AND CD19−, PAX5−, MS4A1−. Hurdle models were used to compare feature counts in each cell subset before and after treatment and obtain a DEG list. Gene set enrichment with the Partek tool was performed on all significant (Adj p<0.05) DEG.

An alternative analysis based on the R package Seurat (version 4.3.0.1) (satijalab.org/seurat/) for single-cell expression analysis, as also performed to ensure the accuracy of the Partek analysis and conclusions. The reads for each sample were loaded using ReadParseBio function and identified Rhesus macaque mitochondrial reads using the scCustomize package (version 1.1.1) (doi.org/10.5281/zenodo.5706430). cells with £200 genes and for mitochondrial gene fraction were filtered out dependent on the sample. All samples were normalized using SCTransform(140). For downstream analysis, the focus was on T cells, defined as expressing CD3 and not CD19 nor PAX5 nor MS4A1. Integration was performed according to the Satija lab's integration workflow (satijalab.org/seurat/articles/integration_introduction.html). Once the datasets were integrated, data was generated dimensionality reductions by PCA and UMAP, using the first principal components for UMAP generation and a cluster size of 0.5. Next, the data was further subset to isolate CD4+ T cells, defined as T cells expressing CD4, and separately CD8+ T cells, defined as T cells expressing either CD8A or CD8B. the differentially expressed genes were calculated comparing W37 to W35 using Wilcoxon matched pairs test. Together, broad agreement was observed with the independent analysis performed with Partek described above, although there were some differences. In CD4+ T cells, 2,022 DEGs with a log 2FC of 0.5 (Adj P-value <0.05) was found, while 2,043 DEGs were in CD8+ T cells and 2,339 DEG were in T cells, with 172 downregulated genes in the CD8+ T cells as compared to 187 in the CD4+ T cells and 74 in T cells. These DEGs covered 36% for T cells and 33% for CD4+ T cells from the Partek analysis.

Amongst the AP-1 family genes, the log 2FC for this analysis vs Partek analysis was as follows: JUNB (T cell: −1.06 vs 0.06), JUN (T cell: −0.22 vs 0.21), FOS (T cell: 1.21 vs 0.16), FOSB (T cell: −0.70 vs 0.25), FOSL2 (T cell: 2.16 vs 0.01), MAF (T cell: 3.70 vs 0.01), BATF (T cell: 4.70 vs 0.01), ATF3 (T cell: 0.60 vs 0.06), MAFF (T cell: 3.97 vs 0.00). Although b2 macroglobulin and RPL13 were not differentially expressed in this analysis, the fold change was in the same direction as Partek analysis (T cell: log 2FC 1.77 and log 2FC 0.03 of SCT normalized counts, respectively). While STAT1 was upregulated in CD4+ T cells (log 2FC 0.88), it was downregulated in CD8+ T cells (log 2FC−0.72). Although TCF7 was not statistically significantly differentially expressed in CD8+ T cells, the W37 vs W35 SCT normalized expression of TCF7 was in the same direction as Partek analysis (log 2FC−0.79).

Bulk and scRNAseq Analysis

For bulk RNAseq, snap frozen PBMC pellets from BC1, 1 hr after first Gal dose and AC1 were used for RNA extraction with the RNeasy kit with on column DNA digestion (Qiagen). Library preparation was performed using TruSeq Stranded Total RNA with Ribo-Zero Globin and sequencing was done with an Illumina HiSeq4000 with >20M reads/samples. Sequencing data was demultiplexed and trimmed using Trimmomatic v0.36 to remove adapters and low-quality reads. Trimmed reads were aligned to the Mmul10 reference genome and transcripts quantified using the Hisat2-StringTie pipeline(138). Differential gene expression analysis using the quantified gene transcripts was performed with DESeq2 R package(139) comparing the samples attained before and after galunisertib treatment and controlling for intra-animal autocorrelation. Differentially expressed genes (DEGs) were analyzed by functional enrichment analysis and gene set enrichment analysis (GSEA) to identify specific pathways and molecular processes altered by galunisertib.

The Parse pipeline and Partek software were. For bulk RNAseq features with <100 counts were removed, data normalized and DESeq2 was used to obtain a DEG list. Genes with a false discovery rate (FDR)-adjusted p-value ≤0.05 and absolute log 2 fold-change (FC) (compared to BC1) above 2 were defined as significantly differentially expressed (DEG). For scRNAseq analysis cells isolated from lymph nodes before (BC1) and after (AC1) galuniertib were fixes with the Parse fixation kit, barcoded and sequenced at the NUseq Core. The Partek software was used for scRNAseq analysis. Cells with 400-8000 features, excluding features with 0 reads in >99.99 cells, were included. Scran deconvolution was used for normalization and cell classification was based on gene expression. Hurdle models were used to compare DEGs in each cell subset before and after galunisertib.

### Barcode Sequencing and Analysis

RNA isolation from plasma was performed using a QIAamp Viral RNA mini kit (Qiagen). Complementary DNA (cDNA) was generated with Superscript III reverse transcriptase (Invitrogen) and an SIV-specific reverse primer (Vpr.cDNA3: 5′-CAG GTT GGC CGA TTC TGG AGT GGA TGC-3′). The cDNA was quantified via qRT-PCR using the primers VpxF1 5′-CTA GGG GAA GGA CAT GGG GCA GG-3′ and VprR1 5′-CCA GAA CCT CCA CTA CCC ATT CATC with labeled probe (ACC TCC AGA AAA TGA AGG ACC ACA AAG GG). Prior to sequencing, PCR was performed with VpxF1 and vprR1 primers combined with either the F5 or F7 Illumina adaptors containing unique 8-nucleotide index sequences for multiplexing. PCR was performed using High Fidelity Platinum Taq (ThermoFisher) under the following conditions: 94° C., 2 min; 40 Å˜ (94° C., 15 s; 60° C., 1.5 min; 68° C., 30 s); 68° C., 5 min. The multiplexed samples and Phi X 174 library were prepared via standard protocols and sequenced on a MiSeq instrument (Illumina).

For viral population analysis, Shannon Entropy of the viral barcodes present in each sample was used to measure the diversity of the viral populations. Shannon Entropy was calculated in R v4.2.2 using the barcode frequencies applying the formula: Sh=Σ(−Pi*log 2 (Pi)); where Sh is Shannon Entropy calculated for each sample, and Pi is the frequency of each barcode. To test for changes in Sh before and after Galunisertib treatment, we fitted linear mixed effects models for each compartment and treatment cycle assuming a log-normal distribution of Sh, using before and after treatment time point as predictor and controlling for Sh value at peak viremia, and within animal correlation, the latter as a random effect. Modeling was performed using the lme4 v1.1-34 R package.

### Plasma Cytokine and T Cell Responses

Cytokines in plasma (at 1:2 dilution) were measured using the NHP Cytokine 24-Plex kit by Meso Scale Diagnostics (MSD) according to manufacturer instructions. Frozen PBMC collected at week 6 post-infection (pre-ART), right before the first galunisertib administration (BC1), at the end of cycle 1 (AC1) and at the end of cycle 3 (AC3) were thawed in AIM V medium (ThermoFisher) with benzonase (Sigma) and plated on a FluoroSpot (CTL) plate pre-activated with 70% Ethanol and IFN-γ, TNF-α and IL-2 capture solution. Gag, pol, and env 15-mer peptides (NIH AIDS Reagents program) were prepared at two times the final concentration of 2.5 μg/ml with co-stimulatory reagents anti-CD28 10 μg/ml and anti-CD49d 10 μg/ml and added to the cells in CTL-Test™ Medium. Parallel positive 587 control of PMA (20 ng/ml) and ionomycin (200 ng/mL) or mock DMSO solution was also plated with the stimulatory reagents. PBMCs were added at 300,000 cells per well. After 24 hrs, the plate was washed, and incubated with detection and tertiary solutions and shipped to CTL for scanning and QC.

### Statistics

GraphPad Prism v10, R and Python were used for statistical analysis and data visualization. Wilcoxon matched-pairs test and repeated measures ANOVA or mixed effect analysis (when the data set had missing data) were used to compare the different virological and immunological variables between baseline (BC1) and a single or multiple post-galunisertib time points. In Prism the mixed model uses a compound symmetry covariance matrix, and is fit using Restricted Maximum Likelihood (REML). Cytokine data from MSD assay were first Log transformed, then normalized by subcolumn/factor in percentage with 0% as smallest value and 100% larger value in each dataset. Each factor was analyzed separately with ANOVA for repeated measures and Dunn's multiple comparison's post-hoc test and together by principal component analysis (PCA). Holm-Sidak test was used for multiple comparison correction in all cases, but FlowSOM cluster comparison where the FDR method was used. Pearson r coefficient was calculated for pairwise tests of association in a correlation matrix with selected variables. For RNAseq analysis see above and supplemental methods. For viral population analysis, Shannon Entropy of the viral barcodes present in each sample was used to measure the diversity of the viral populations (see supplemental Methods). Chi-squared tests were used in R to compare barcode composition between before and after cycle time points within a tissue/macaque using paired barcode relative frequencies. Unless otherwise specified p-value <0.05 was considered statistically significant.

### REFERENCES

80. P. Gantner, S. Buranapraditkun, A. Pagliuzza, C. Dufour, M. Pardons, J. L. Mitchell, E. Kroon, C. Sacdalan, N. Tulmethakaan, S. Pinyakorn, M. L. Robb, N. Phanuphak, J. Ananworanich, D. Hsu, S. Vasan, L. Trautmann, R. Fromentin, N. Chomont, HIV rapidly targets a diverse pool of CD4(+) T cells to establish productive and latent infections. Immunity 56, 653-668 e655 (2023).
81. J. B. Whitney, A. L. Hill, S. Sanisetty, P. Penaloza-MacMaster, J. Liu, M. Shetty, L. Parenteau, C. Cabral, J. Shields, S. Blackmore, J. Y. Smith, A. L. Brinkman, L. E. Peter, S. I. Mathew, K. M. Smith, E. N. Borducchi, D. I. Rosenbloom, M. G. Lewis, J. Hattersley, B. Li, J. Hesselgesser, R. Geleziunas, M. L. Robb, J. H. Kim, N. L. Michael, D. H. Barouch, Rapid seeding of the viral reservoir prior to SIV viraemia in rhesus monkeys. Nature 512, 74-77 (2014).
82. D. Persaud, Y. Zhou, J. M. Siliciano, R. F. Siliciano, Latency in human immunodeficiency virus type 1 infection: no easy answers. J Virol 77, 1659-1665 (2003).
83. D. M. Margolis, N. M. Archin, Proviral Latency, Persistent Human Immunodeficiency Virus Infection, and the Development of Latency Reversing Agents. The Journal of infectious diseases 215, S111-S118 (2017).
84. T. A. Wagner, S. Mclaughlin, K. Garg, C. Y. Cheung, B. B. Larsen, S. Styrchak, H. C. Huang, P. T. Edlefsen, J. I. Mullins, L. M. Frenkel, HIV latency. Proliferation of cells with HIV integrated into cancer genes contributes to persistent infection. Science 345, 570-573 (2014).
85. L. B. Cohn, I. T. Silva, T. Y. Oliveira, R. A. Rosales, E. H. Parrish, G. H. Learn, B. H. Hahn, J. L. Czartoski, M. J. McElrath, C. Lehmann, F. Klein, M. Caskey, B. D. Walker, J. D. Siliciano, R. F. Siliciano, M. Jankovic, M. C. Nussenzweig, HIV-1 integration landscape during latent and active infection. Cell 160, 420-432 (2015).
86. K. B. Einkauf, M. R. Osborn, C. Gao, W. Sun, X. Sun, X. Lian, E. M. Parsons, G. T. Gladkov, K. W. Seiger, J. E. Blackmer, C. Jiang, S. A. Yukl, E. S. Rosenberg, X. G. Yu, M. Lichterfeld, Parallel analysis of transcription, integration, and sequence of single HIV-1 proviruses. Cell 185, 266-282 e215 (2022).
87. X. Lian, K. W. Seiger, E. M. Parsons, C. Gao, W. Sun, G. T. Gladkov, I. C. Roseto, K. B. Einkauf, M. R. Osborn, J. M. Chevalier, C. Jiang, J. Blackmer, M. Carrington, E. S. Rosenberg, M. M. Lederman, D. K. McMahon, R. J. Bosch, J. M. Jacobson, R. T. Gandhi, M. J. Peluso, T. W. Chun, S. G. Deeks, X. G. Yu, M. Lichterfeld, Progressive transformation of the HIV-1 reservoir cell profile over two decades of antiviral therapy. Cell Host Microbe 31, 83-96 e85 (2023). 88. E. Abner, A. Jordan, HIV “shock and kill” therapy: In need of revision. Antiviral Res 166, 19-34 (2019).
89. E. N. Borducchi, C. Cabral, K. E. Stephenson, J. Liu, P. Abbink, D. Ng′ang′a, J. P. Nkolola,

A. L. Brinkman, L. Peter, B. C. Lee, J. Jimenez, D. Jetton, J. Mondesir, S. Mojta, A. Chandrashekar, K. Molloy, G. Alter, J. M. Gerold, A. L. Hill, M. G. Lewis, M. G. Pau, H. Schuitemaker, J. Hesselgesser, R. Geleziunas, J. H. Kim, M. L. Robb, N. L. Michael, D. H. Barouch, Ad26/MVA therapeutic vaccination with TLR7 stimulation in SIV-infected rhesus monkeys. Nature 540, 284-287 (2016).
90. C. C. Nixon, M. Mavigner, G. C. Sampey, A. D. Brooks, R. A. Spagnuolo, D. M. Irlbeck, C. Mattingly, P. T. Ho, N. Schoof, C. G. Cammon, G. K. Tharp, M. Kanke, Z. Wang, R. A. Cleary, A. A. Upadhyay, C. De, S. R. Wills, S. D. Falcinelli, C. Galardi, H. Walum, N. J. Schramm, J. Deutsch, J. D. Lifson, C. M. Fennessey, B. F. Keele, S. Jean, S. Maguire, B. Liao, E. P. Browne, R. G. Ferris, J. H. Brehm, D. Favre, T. H. Vanderford, S. E. Bosinger, C. D. Jones, J. P. Routy, N. M. Archin, D. M. Margolis, A. Wahl, R. M. Dunham, G. Silvestri, A. Chahroudi, J. V. Garcia, Systemic HIV and SIV latency reversal via non-canonical NF-kappaB signalling in vivo. Nature 578, 160-165 (2020).
91. D. O'Sullivan, The metabolic spectrum of memory T cells. Immunol Cell Biol 97, 636-646 (2019).
92. J. M. Crater, D. F. Nixon, R. L. Furler O'Brien, HIV-1 replication and latency are balanced by mTOR-driven cell metabolism. Front Cell Infect Microbiol 12, 1068436 (2022).
93. E. Besnard, S. Hakre, M. Kampmann, H. W. Lim, N. N. Hosmane, A. Martin, M. C. Bassik, E. Verschueren, E. Battivelli, J. Chan, J. P. Svensson, A. Gramatica, R. J. Conrad, M. Ott, W. C. Greene, N. J. Krogan, R. F. Siliciano, J. S. Weissman, E. Verdin, The mTOR Complex Controls HIV Latency. Cell Host Microbe 20, 785-797 (2016).
94. H. J. Barbian, M. S. Seaton, S. D. Narasipura, J. Wallace, R. Rajan, B. E. Sha, L. Al-Harthi, beta-catenin regulates HIV latency and modulates HIV reactivation. PLOS Pathog 18, e1010354 (2022).
95. F. Gounari, K. Khazaie, TCF-1: a maverick in T cell development and function. Nat Immunol 23, 671-678 (2022).
96. J. C. Valle-Casuso, M. Angin, S. Volant, C. Passaes, V. Monceaux, A. Mikhailova, K. Bourdic, V. Avettand-Fenoel, F. Boufassa, M. Sitbon, O. Lambotte, M. I. Thoulouze, M. Muller-Trutwin, N. Chomont, A. Saez-Cirion, Cellular Metabolism Is a Major Determinant of HIV-1 Reservoir Seeding in CD4(+) T Cells and Offers an Opportunity to Tackle Infection. Cell Metab 29, 611-626 e615 (2019).
97. S. A. Oh, M. O. Li, TGF-beta: guardian of T cell function. Journal of immunology 191, 3973-3979 (2013).
98. S. S. Gabriel, C. Tsui, D. Chisanga, F. Weber, M. Llano-Leon, P. M. Gubser, L. Bartholin, F. Souza-Fonseca-Guimaraes, N. D. Huntington, W. Shi, D. T. Utzschneider, A. Kallies, Transforming growth factor-beta-regulated mTOR activity preserves cellular metabolism to maintain long-term T cell responses in chronic infection. Immunity 54, 1698-1714 e1695 (2021).
99. Y. Hu, W. H. Hudson, H. T. Kissick, C. B. Medina, A. P. Baptista, C. Ma, W. Liao, R. N. Germain, S. J. Turley, N. Zhang, R. Ahmed, TGF-beta regulates the stem-like state of PD-1+ TCF-1+ virus-specific CD8 T cells during chronic infection. J Exp Med 219, (2022).
100. C. Ma, N. Zhang, Transforming growth factor-beta signaling is constantly shaping memory T-cell population. Proc Natl Acad Sci USA 112, 11013-11017 (2015).
101. S. Viel, A. Marcais, F. S. Guimaraes, R. Loftus, J. Rabilloud, M. Grau, S. Degouve, S. Djebali, A. Sanlaville, E. Charrier, J. Bienvenu, J. C. Marie, C. Caux, J. Marvel, L. Town, N. D. Huntington, L. Bartholin, D. Finlay, M. J. Smyth, T. Walzer, TGF-beta inhibits the activation and functions of NK cells by repressing the mTOR pathway. Sci Signal 9, ra19 (2016).
102. G. M. Lewis, E. J. Wehrens, L. Labarta-Bajo, H. Streeck, E. I. Zuniga, TGF-beta receptor maintains CD4 T helper cell identity during chronic viral infections. J Clin Invest 126, 3799-3813 (2016).
103. A. P. Nath, A. Braun, S. C. Ritchie, F. R. Carbone, L. K. Mackay, T. Gebhardt, M. Inouye, Comparative analysis reveals a role for TGF-beta in shaping the residency-related transcriptional signature in tissue-resident memory CD8+ T cells. PLOS One 14, e0210495 (2019).
104. N. Zhang, M. J. Bevan, Transforming growth factor-beta signaling controls the formation and maintenance of gut-resident memory T cells by regulating migration and retention. Immunity 39, 687-696 (2013).
105. T. Hirai, Y. Yang, Y. Zenke, H. Li, V. K. Chaudhri, J. S. De La Cruz Diaz, P. Y. Zhou, B. A. Nguyen, L. Bartholin, C. J. Workman, D. W. Griggs, D. A. A. Vignali, H. Singh, D. Masopust, D. H. Kaplan, Competition for Active TGFbeta Cytokine Allows for Selective Retention of Antigen-Specific Tissue-Resident Memory T Cells in the Epidermal Niche. Immunity 54, 84-98 e85 (2021).
106. C. Larson, B. Oronsky, C. A. Carter, A. Oronsky, S. J. Knox, D. Sher, T. R. Reid, TGF-beta: a master immune regulator. Expert Opin Ther Targets 24, 427-438 (2020).
107. S. Samer, Y. Thomas, M. Arainga, C. Carter, L. M. Shirreff, M. S. Arif, J. M. Avita, I. Frank, M. D. McRaven, C. T. Thuruthiyil, V. B. Heybeli, M. R. Anderson, B. Owen, A. Gaisin, D. Bose, L. M. Simons, J. F. Hultquist, J. Arthos, C. Cicala, I. Sereti, P. J. Santangelo, R. Lorenzo-Redondo, T. J. Hope, F. J. Villinger, E. Martinelli, Blockade of TGF-beta signaling reactivates HIV-1/SIV reservoirs and immune responses in vivo. JCI Insight 7, (2022).
108. S. Bergstresser, D. A. Kulpa, TGF-beta Signaling Supports HIV Latency in a Memory CD4+ T Cell Based In Vitro Model. Methods Mol Biol 2407, 69-79 (2022).
109. A. Wick, A. Desjardins, C. Suarez, P. Forsyth, I. Gueorguieva, T. Burkholder, A. L. Cleverly, S. T. Estrem, S. Wang, M. M. Lahn, S. C. Guba, D. Capper, J. Rodon, Phase 1b/2a study of galunisertib, a small molecule inhibitor of transforming growth factor-beta receptor I, in combination with standard temozolomide-based radiochemotherapy in patients with newly diagnosed malignant glioma. Invest New Drugs 38, 1570-1579 (2020).
110. S. Samer, Y. Thomas, M. Arainga, C. Carter, L. Shirreff, M. Arif, J. Avita, I. Frank, M. McRaven, C. Thuruthiyil, V. Heybeli, M. Anderson, B. Owen, A. Gaisin, D. Bose, L. Simons, J. Hultquist, J. Arthos, C. Cicala, I. Sereti, P. Santangelo, R. Lorenzo-Redondo, T. Hope, F. Villinger, E. Martinelli, Blockade of TGF-β signaling reactivates HIV-1/SIV reservoirs and immune responses <em>in vivo</em>. bioRxiv, 2022.2005.2013.489595 (2022).
111. S. J. Im, B. T. Konieczny, W. H. Hudson, D. Masopust, R. Ahmed, PD-1+ stemlike CD8 T cells are resident in lymphoid tissues during persistent LCMV infection. Proc Natl Acad Sci USA 117, 4292-4299 (2020).
112. S. Van Gassen, B. Callebaut, M. J. Van Helden, B. N. Lambrecht, P. Demeester, T. Dhaene, Y. Saeys, FlowSOM: Using self-organizing maps for visualization and interpretation of cytometry data. Cytometry A 87, 636-645 (2015).
113. J. A. Rebhahn, S. A. Quataert, G. Sharma, T. R. Mosmann, SwiftReg cluster registration automatically reduces flow cytometry data variability including batch effects. Commun Biol 3, 218 (2020).
114. M. J. Blackburn, M. Zhong-Min, F. Caccuri, K. Mckinnon, L. Schifanella, Y. Guan, G. Gorini, D. Venzon, C. Fenizia, N. Binello, S. N. Gordon, C. J. Miller, G. Franchini, M. Vaccari, Regulatory and Helper Follicular T Cells and Antibody Avidity to Simian Immunodeficiency Virus Glycoprotein 120. J Immunol 195, 3227-3236 (2015).
115. S. Helmold Hait, D. A. Vargas-Inchaustegui, T. Musich, V. Mohanram, I. Tuero, D. J. Venzon, J. Bear, M. Rosati, M. Vaccari, G. Franchini, B. K. Felber, G. N. Pavlakis, M. Robert-Guroff, Early T Follicular Helper Cell Responses and Germinal Center Reactions Are Associated with Viremia Control in Immunized Rhesus Macaques. J Virol 93, (2019).
116. Y. Zhou, B. Zhou, L. Pache, M. Chang, A. H. Khodabakhshi, O. Tanaseichuk, C. Benner, S. K. Chanda, Metascape provides a biologist-oriented resource for the analysis of systems798 level datasets. Nat Commun 10, 1523 (2019).
117. Z. Qiu, T. H. Chu, B. S. Sheridan, TGF-beta: Many Paths to CD103(+) CD8 T Cell Residency. Cells 10, (2021).
118. H. S. Lim, P. Qiu, Quantifying Cell-Type-Specific Differences of Single-Cell Datasets Using Uniform Manifold Approximation and Projection for Dimension Reduction and Shapley Additive explanations. J Comput Biol 30, 738-750 (2023).
119. X. Liao, N. Sharma, F. Kapadia, G. Zhou, Y. Lu, H. Hong, K. Paruchuri, G. H. Mahabeleshwar, E. Dalmas, N. Venteclef, C. A. Flask, J. Kim, B. W. Doreian, K. Q. Lu, K. H. Kaestner, A. Hamik, K. Clement, M. K. Jain, Kruppel-like factor 4 regulates macrophage polarization. J Clin Invest 121, 2736-2749 (2011).
120. M. A. Moso, J. L. Anderson, S. Adikari, L. R. Gray, G. Khoury, J. J. Chang, J. C. Jacobson, A. M. Ellett, W. J. Cheng, S. Saleh, J. J. Zaunders, D. F. J. Purcell, P. U. Cameron, M. J. Churchill, S. R. Lewin, H. K. Lu, HIV latency can be established in proliferating and nonproliferating resting CD4+ T cells in vitro: implications for latency reversal. AIDS 33, 199-209 (2019).
121. J. Neidleman, X. Luo, J. Frouard, G. Xie, F. Hsiao, T. Ma, V. Morcilla, A. Lee, S. Telwatte, R. Thomas, W. Tamaki, B. Wheeler, R. Hoh, M. Somsouk, P. Vohra, J. Milush, K. S. James, N. M. Archin, P. W. Hunt, S. G. Deeks, S. A. Yukl, S. Palmer, W. C. Greene, N. R. Roan, Phenotypic analysis of the unstimulated in vivo HIV CD4 T cell reservoir. Elife 9, (2020).
122. A. Chitrakar, M. Sanz, S. B. Maggirwar, N. Soriano-Sarabia, HIV Latency in Myeloid Cells: Challenges for a Cure. Pathogens 11, (2022).
123. E. L. Pearce, Metabolism in T cell activation and differentiation. Curr Opin Immunol 22, 314-320 (2010).
124. H. Valdez, M. M. Lederman, Cytokines and cytokine therapies in HIV infection. AIDS Clin Rev, 187-228 (1997).
125. R. J. Salmond, Regulation of T Cell Activation and Metabolism by Transforming Growth Factor-Beta. Biology (Basel) 12, (2023).
126. S. Regis, A. Dondero, F. Caliendo, C. Bottino, R. Castriconi, NK Cell Function Regulation by TGF-beta-Induced Epigenetic Mechanisms. Front Immunol 11, 311 (2020).

127. V. Zaiatz-Bittencourt, D. K. Finlay, C. M. Gardiner, Canonical TGF-beta Signaling Pathway Represses Human NK Cell Metabolism. J Immunol 200, 3934-3941 (2018).
128. L. Das, A. D. Levine, TGF-beta inhibits IL-2 production and promotes cell cycle arrest in TCR-activated effector/memory T cells in the presence of sustained TCR signal transduction. J Immunol 180, 1490-1498 (2008).
129. W. Chen, TGF-beta Regulation of T Cells. Annu Rev Immunol 41, 483-512 (2023).
130. T. Yamazaki, A. J. Gunderson, M. Gilchrist, M. Whiteford, M. X. Kiely, A. Hayman, D. O'Brien, R. Ahmad, J. V. Manchio, N. Fox, K. McCarty, M. Phillips, E. Brosnan, G. Vaccaro, R. Li, M. Simon, E. Bernstein, M. McCormick, L. Yamasaki, Y. Wu, A. Drokin, T. Carnahan, Y. To, W. L. Redmond, B. Lee, J. Louie, E. Hansen, M. C. Solhjem, J. Cramer, W. J. Urba, M. J. Gough, M. R. Crittenden, K. H. Young, Galunisertib plus neoadjuvant chemoradiotherapy in patients with locally advanced rectal cancer: a single-arm, phase 2 trial. Lancet Oncol 23, 1189-1200 (2022).
131. R. K. Kelley, E. Gane, E. Assenat, J. Siebler, P. R. Galle, P. Merle, I. O. Hourmand, A. Cleverly, Y. Zhao, I. Gueorguieva, M. Lahn, S. Faivre, K. A. Benhadji, G. Giannelli0, A Phase 2 Study of Galunisertib (TGF-beta1 Receptor Type I Inhibitor) and Sorafenib in Patients With Advanced Hepatocellular Carcinoma. Clin Transl Gastroenterol 10, e00056 (2019).
132. D. Melisi, R. Garcia-Carbonero, T. Macarulla, D. Pezet, G. Deplanque, M. Fuchs, J. Trojan, H. Oettle, M. Kozloff, A. Cleverly, C. Smith, S. T. Estrem, I. Gueorguieva, M. M. F. Lahn, A. Blunt, K. A. Benhadji, J. Tabernero, Galunisertib plus gemcitabine vs. gemcitabine for first-line treatment of patients with unresectable pancreatic cancer. Br J Cancer 119, 1208-1214 (2018).
133. E. J. Fray, F. Wu, F. R. Simonetti, C. Zitzmann, N. Sambaturu, C. Molina-Paris, A. M. Bender, P. T. Liu, J. D. Ventura, R. W. Wiseman, D. H. O'Connor, R. Geleziunas, T. Leitner, R. M. Ribeiro, A. S. Perelson, D. H. Barouch, J. D. Siliciano, R. F. Siliciano, Antiretroviral therapy reveals triphasic decay of intact SIV genomes and persistence of ancestral variants. Cell Host Microbe 31, 356-372 e355 (2023).
134. G. Escobar, D. Mangani, A. C. Anderson, T cell factor 1: A master regulator of the T cell response in disease. Sci Immunol 5, (2020).
135. R. L. Rutishauser, C. D. T. Deguit, J. Hiatt, F. Blaeschke, T. L. Roth, L. Wang, K. A. Raymond, C. E. Starke, J. C. Mudd, W. Chen, C. Smullin, R. Matus-Nicodemos, R. Hoh, M. Krone, F. M. Hecht, C. D. Pilcher, J. N. Martin, R. A. Koup, D. C. Douek, J. M. Brenchley, R. P. Sekaly, S. K. Pillai, A. Marson, S. G. Deeks, J. M. McCune, P. W. Hunt, TCF-1 regulates HIV-specific CD8+ T cell expansion capacity. JCI Insight 6, (2021).
136. Y. S. Choi, J. A. Gullicksrud, S. Xing, Z. Zeng, Q. Shan, F. Li, P. E. Love, W. Peng, H. H. Xue, S. Crotty, LEF-1 and TCF-1 orchestrate T(FH) differentiation by regulating differentiation circuits upstream of the transcriptional repressor Bcl6. Nat Immunol 16, 980-990 (2015).
137. S. G. Zheng, J. Wang, P. Wang, J. D. Gray, D. A. Horwitz, IL-2 is essential for TGF-beta to convert naive CD4+CD25− cells to CD25+ Foxp3+ regulatory T cells and for expansion of these cells. J Immunol 178, 2018-2027 (2007).
138. M. Pertea, D. Kim, G. M. Pertea, J. T. Leek, S. L. Salzberg, Transcript-level expression analysis of RNA-seq experiments with HISAT, StringTie and Ballgown. Nat Protoc 11, 1650-1667 (2016).
139. M. I. Love, W. Huber, S. Anders, Moderated estimation of fold change and dispersion for RNA-seq data with DESeq2. Genome Biol 15, 550 (2014).
140. C. Hafemeister, R. Satija, Normalization and variance stabilization of single-cell RNAseq data using regularized negative binomial regression. Genome Biol 20, 296 (2019).

We claim:

1. A method of treating a latent immunodeficiency virus infection in immune cells in a subject receiving antiretroviral therapy comprising administering a TGF-β inhibitor to the subject.

2. The method of claim 2, wherein the TGF-β inhibitor is galunisertib.

3. The method of claim 1, wherein the immunodeficiency virus is Human Immunodeficiency Virus (HIV).

4. The method of claim 1, wherein the antiretroviral therapy suppresses active immunodeficiency virus in the subject.

5. The method of claim 4, wherein the TGF-β inhibitor is administered after the antiretroviral therapy suppresses active immunodeficiency virus in the subject

6. The method of claim 1, wherein the TGF-β inhibitor activates the latent immunodeficiency virus in the infected immune cells.

7. The method of claim 1, wherein the TGF-β inhibitor reduces immunodeficiency provirus in the immune cells infected with the virus.

8. The method of claim 1, wherein the TGF-β inhibitor stimulates immune cells to effector immune cells in the subject.

9. The method of claim 8, wherein the effector immune cells are T cells, B cells, NK cells, and/or macrophages.

10. The method of claim 9, wherein the effector immune cells are T cells expressing CD4.

11. The method of claim 10, wherein the T cells have increased expression of CD95 over naïve cells.

12. The method of claim 10, wherein the T cells have decreased expression of TCF1 over naïve cells.

13. A method for activation of latent immunodeficiency virus in immune cells of a subject having an immunodeficiency virus infection treated with antiretroviral therapy comprising contacting the immune cells with a TGF-β inhibitor.

14. The method of claim 13, wherein the TGF-β inhibitor is galunisertib.

15. The method of claim 13, wherein the immunodeficiency virus is a Human Immunodeficiency Virus.

16. The method of claim 13, wherein the TGF-β inhibitor is administered while the subject is receiving the antiretroviral therapy.

17. The method of claim 13, wherein the antiretroviral therapy suppresses active immunodeficiency virus in the subject.

18. The method of claim 17, wherein the TGF-β inhibitor is administered after the antiretroviral therapy suppresses active immunodeficiency virus in the subject.

19. The method of claim 18, wherein the TGF-β inhibitor activates the latent immunodeficiency virus in the infected immune cells.

20. The method of claim 18, wherein the TGF-β inhibitor stimulates immune cells to effector immune cells in the subject.
